(19) United States
(12) Patent Application Publication
(10) Pub. No.: US 2025/0041303 A1
(43) Pub. Date: Feb. 6, 2025

(54) COMBINATION THERAPY FOR CANCER TREATMENT

(71) Applicant: INSTITUTE FOR CANCER RESEARCH D/B/A THE RESEARCH INSTITUTE OF FOX CHASE CANCER CENTER, Philadelphia, PA (US)

(72) Inventor: James S. Duncan, Philadelphia, PA (US)

(21) Appl. No.: 18/715,013

(22) PCT Filed: Nov. 29, 2022
(86) PCT No.: PCT/US2022/080567

§ 371 Date: May 30, 2024

Related U.S. Application Data
(60) Provisional application No. 63/284,218, filed on Nov. 30, 2021.

(51) Int. Cl. A61K 31/517; A61K 31/453; A61K 45/06; A61P 35/02

(52) U.S. Cl. CPC A61K 31/517; A61K 31/453; A61K 45/06; A61P 35/02

(57) ABSTRACT

Pharmaceutical compositions comprising: one or more PROTAC therapeutic agents; and one or more kinase inhibitors, one or more ABC transporter inhibitors, or one or more dual ABC transporter/kinase inhibitors, or any combination thereof; and methods of treating cancer in a human by administering: one or more PROTAC therapeutic agents; and one or more kinase inhibitors, one or more ABC transporter inhibitors, or one or more dual ABC transporter/kinase inhibitors, or any combination thereof, are described herein.

### REFERENCE TO GOVERNMENT GRANTS

This invention was made with government support under CA211670 awarded by the National Institutes of Health. The government has certain rights in the invention.

### FIELD

The present disclosure is directed to pharmaceutical compositions comprising: one or more proteolysis targeting chimera (PROTAC) therapeutic agents; and one or more kinase inhibitors, one or more ABC transporter inhibitors, or one or more dual ABC transporter/kinase inhibitors, or any combination thereof; and methods of treating cancer in a human by administering: one or more PROTAC therapeutic agents; and one or more kinase inhibitors, one or more ABC transporter inhibitors, or one or more dual ABC transporter/kinase inhibitors, or any combination thereof, are described herein.

### BACKGROUND

PROTACs (Proteolysis-Targeting Chimeras) have emerged as a revolutionary new class of drugs that utilize the cancer cells' own protein destruction machinery to selectively degrade essential tumor drivers (Neklesa et al., Pharmacol. Ther., 2017, 174, 138-144). PROTACs are small molecules with two functional ends, a small-molecule end that binds to the protein of interest and the other end that binds to an E3 ubiquitin ligase (Bondeson et al., Cell Chem. Biol., 2018, 25, 78-87; and Lai et al., Nat. Rev. Drug Discov., 2017, 16, 101-114). The PROTAC component recruits the ubiquitin ligase to the target protein, leading to its ubiquitination and subsequent degradation by the proteasome. Benefits of PROTACs include development of drugs against previously undruggable drug targets, non-reliance on catalytic activity for degradation, and they do not require high affinity for the drug target to achieve protein degradation. Additionally, low doses of PROTACs can be highly effective at inducing degradation, which can reduce off-target toxicity associated with high-dosing of traditional inhibitors. PROTACs have been developed for a variety of cancer targets including oncogenic kinases (Yu et al., Targeting Protein Kinases Degradation by PROTACs, Frontiers in Chemistry 9, 2021), epigenetic targets (Vogelmann et al., Curr. Opin. Chem. Biol., 2020, 57, 8-16) and recently KRAS^{G12C} proteins (Bond et al., ACS Central Science, 2020, 6, 1367-1375). PROTACs targeting the androgen receptor or estrogen receptor are avidly being evaluated in clinical trials for prostate (NCT03888612) or breast cancers (NCT04072952), respectively. Drug resistance, however, represents a significant therapeutic challenge for the treatment of cancer.

### SUMMARY

The present disclosure provides pharmaceutical compositions comprising: one or more PROTAC therapeutic agents; and one or more kinase inhibitors, one or more ABC transporter inhibitors, or one or more dual ABC transporter/kinase inhibitors, or any combination thereof.

The present disclosure also provides methods for augmenting the therapeutic effect of a human undergoing cancer treatment with a PROTAC therapeutic agent, the method comprising administering one or more kinase inhibitors, one or more ABC transporter inhibitors, or one or more dual ABC transporter/kinase inhibitors, or any combination thereof, to the human.

The present disclosure also provides methods of treating cancer in a human in need thereof, the method comprising administering to the human: one or more PROTAC therapeutic agents; and one or more kinase inhibitors, one or more ABC transporter inhibitors, or one or more dual ABC transporter/kinase inhibitors, or any combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows proteomics characterization of degrader-resistant cancer cell lines. Panel A shows workflow for identifying protein targets upregulated in degrader-resistant cancer cells; single-run proteome analysis was performed and changes in protein levels amongst parent and resistant cells determined by label-free quantitation. Panels B and C show A1847 cells acquire resistance to dBET6 or Thal SNS 032; parental and dBET6 or Thal SNS 032-resistant cells were treated with escalating doses of dBET6 (Panel B) or Thal SNS 032 (Panel C) for 5 days and cell viability assessed by CellTiter-Glo; degrader-R treated cell viabilities normalized to DMSO treated degrader-R cells; data present in Panel B and Panel C are triplicate experiments SD. *p≤0.05 by student's t-test. Panels D and E show escalating doses of degraders fails to promote degradation of protein target in degrader-resistant cells; A1847 parental, dBET6-R (Panel D) or Thal-R (Panel E) were treated with escalating doses of dBET6 (0, 0.039, 0.156, 0.625, 2.5 or 10 μM) or Thal SNS 032 (0, 0.039, 0.156, 0.625, or 2.5 μM) for 24 hours and degrader targets and downstream signaling determined by western blot; blots are representative of 3 independent blots. Panels F and G show volcano plots depicting proteins elevated or reduced in dBET6-R (Panel F) or Thal-R (Panel G) relative to parental A1847 cells; differences in protein log 2 LFQ intensities amongst degrader-resistant and parental cells were determined by paired t-test Benjamini-Hochberg adjusted P values at FDR of <0.05 using Perseus software. Panels H and I show the top 10 upregulated proteins in dBET6-R (Panel H) or Thal-R (Panel I) relative to parental A1847 cells. Panels J and K show bar graphs depicting ABCB1 log 2 LFQ values comparing dBET6-R (Panel J) or Thal-R (Panel K) relative to parental A1847 cells; differences in ABCB1 log 2 LFQ intensities amongst degrader-resistant and parental cells were determined by paired t-test Benjamini-Hochberg adjusted P values at FDR of <0.05 using Perseus software.

FIG. 2 shows chronic exposure to degraders induces MDR1 expression and drug efflux activity. Panel A shows ABCB1 mRNA levels are upregulated in degrader-resistant cell lines as determined by qRT-PCR. Panel B shows MDR1 protein levels are upregulated in degrader-resistant cell lines relative to parental cells as determined by immunoblot; blots are representative of 3 independent blots. Panels C, D, and E show confocal fluorescence microscopy of MDR1 protein levels in dBET6-R (Panel C), MZ1-R (Panel D) and Thal-R (Panel E) relative to parental cell lines; MDR1 was detected by immunofluorescence using anti-MDR1 antibodies and nuclear staining by DAPI; images are representative of 3 independent experiments; scale bar=50 μm. Panel F shows a bar graph depicting increased drug efflux activity in dBET6-R, MZ1-R and Thal-R cells relative to parental cells; data present in Panels A and F are triplicate experiments SD; *p<0.05 by student's t-test.

FIG. 3 shows blockade of MDR1 activity re-sensitizes degrader-resistant cells to PROTACs. Panels A and B show degrader-resistant cells acquire dependency on MDR1 for survival; Cell-Titer Glo assay for cell viability of parental, dBET6-R or Thal-R A1847 cells (Panel A) or parental or MZ1-R SUM159 cells (Panel B) transfected with siRNAs targeting ABCB1 or with control siRNA and cultured for 120 hours; triplicate experiments SEM; *p≤0.05 by student's t-test. Panels C, D, and E show knockdown of ABCB1 in dBET6-R (Panel C) or Thal-R (Panel D) A1847 cells or in MZ1-R SUM159 cells (Panel E) promotes degradation of PROTAC-targets; A1847 parental, dBET6-R or Thal-R cells were transfected with siRNAs targeting ABCB1 or with control siRNA and proteins measured by western blot; blots are representative of 3 independent blots. Panels F, G, and H show treatment of degrader-resistant cells with tariquidar reduces MDR1 activity; bar graph depicts decreased drug efflux activity in dBET6-R (Panel F) or Thal-R (Panel G) A1847 cells or MZ1-R SUM159 cells (Panel H) relative to parental cells. Panels I, J, and K show degrader-resistant cells exhibit increased sensitivity to MDR1 inhibitors; Cell-Titer Glo assay for cell viability of parental, dBET6-R (Panel I) or Thal-R (Panel J) A1847 cells or parental or MZ1-R SUM159 cells (Panel K) with increasing concentrations of MDR1 inhibitor tariquidar; triplicate experiments SEM; *p≤0.05 by student's t-test. Panels L, M, and N show treatment of parental, dBET6-R (Panel L) or Thal-R (Panel M) A1847 cells or parental or MZ1-R SUM159 cells (Panel N) promotes degradation of PROTAC-targets; A1847 parental, dBET6-R or Thal-R cells or SUM159 parental or MZ1-R cells were treated with tariquidar (0.1 μM) for 24 hours and proteins measured by western blot; blots are representative of 3 independent blots. Panels O and P show MDR1 inhibition blocks development of degrader-resistance. A1847 cells were treated with DMSO, tariquidar (0.1 μM), dBET6 (0.1 μM) or the combination and colony formation assessed following 14-days of treatment (Panel O); SUM159 cells were treated with DMSO, tariquidar (0.1 μM), MZ1 (0.1 μM) or the combination and colony formation assessed following 14-days of treatment (Panel P); colony formation image representative of 3 independent assays. Panel Q shows forced expression of Flag-MDR1 in SUM159 cells; SUM159 cells were transiently transfected with Flag-MDR1 for 72 hours and MDR1 protein expression verified by western blot. Panel R shows forced expression of Flag-MDR1 promotes resistance to dBET6; SUM159 cells expressing Flag-MDR1 were treated with DMSO, MZ1 (0.1 μM), or MZ1 (0.1 μM) and tariquidar (0.1 μM) and colony formation assessed following 14 days of treatment by crystal violet staining; colony formation image representative of 3 independent assays. Panels S and T show MOLT4 cells do not induce ABCB1 expression following chronic exposure to MZ1 that is observed with OVCAR3 and HCT116; ABCB1 expression and protein levels was assessed in parental or MZ1-R cells using qRT-PCR (Panel S) or immunoblot (Panel T); blots are representative of 3 independent blots. Data present in Panels A, B, F, G, I, J, K, and S are triplicate experiments SD; *p≤0.05 by student's t-test.

FIG. 4 shows overexpression of MDR1 conveys intrinsic resistance to degrader therapies in cancer cells. Panel A shows cancer cells resistant to BET protein degraders harbor elevated ABCB1 expression; expression of ABCB1 in cancer cell lines exhibiting sensitivity or resistance to MZ1/dBET6 was queried. Panel B shows MDR1 protein levels in a panel of cancer cell lines as determined by western blot; blots are representative of 3 independent blots. Panel C shows cancer cells overexpressing MDR1 exhibit reduced sensitivity towards Thal SNS 032; cancer cells were treated with escalating doses of Thal SNS 032 for 5 days and cell viability assessed by CellTiter-Glo; GI50 values were determined in Prism software. Panel D shows overexpression of MDR1 reduces PROTAC-mediated degradation efficiency in cancer cells; cancer cells exhibiting different levels of MDR1 were treated with escalating doses of dBET6 or Thal SNS 032 (Thal) for 4 hours and BRD4 or CDK9 protein levels assessed by western blot; blots are representative of 3 independent blots. Panels E and F show combined inhibition of MDR1 improves PROTAC-mediated degradation in MDR1 overexpressing cells; DLD-1 cells were treated with increasing doses of dBET6 alone or in combination with tariquidar (0.1 μM) (Panel E) or increasing doses of Thal SNS 032 alone or in combination with tariquidar (0.1 μM) (Panel F) for 4 hours and BRD4 or CDK9 protein levels assessed by western blot; blots are representative of 3 independent blots. Panels G, H, and I show combining tariquidar and dBET6 exhibits drug synergy in MDR1-overexpressing cells; Cell-Titer Glo assay for cell viability of DLD-1 cells treated with increasing concentrations of dBET6, tariquidar or the combination and bliss synergy scores determined (Panel G); DLD-1 cells were treated with DMSO, tariquidar (0.1 μM), dBET6 (0.1 μM) or the combination and colony formation assessed following 14-days of treatment (Panel H); colony formation image representative of 3 independent assays; Western blot analysis was performed on DLD-1 cells treated with DMSO, tariquidar (0.1 μM), dBET6 (0.1 μM) or the combination for 24 hours (Panel I); blots are representative of 3 independent blots. Panels J, K, and L show combining tariquidar and Thal SNS 032 exhibits drug synergy in MDR1-overexpressing cells; Cell-Titer Glo assay for cell viability of DLD-1 cells treated with increasing concentrations of Thal SNS 032, tariquidar or the combination and bliss synergy scores determined (Panel J); DLD-1 cells were treated with DMSO, tariquidar (0.1 μM), Thal SNS 032 (0.5 μM) or the combination and colony formation assessed following 14-days of treatment (Panel K); colony formation image representative of 3 independent assays; Western blot analysis was performed on DLD-1 cells treated with DMSO, tariquidar (0.1 μM), Thal SNS (0.5 μM) or the combination for 24 hours (Panel L); blots are representative of 3 independent blots. Panels M and N show combining tariquidar with BET degraders enhances growth inhibition of MDR1-overexpressing cell lines HCT-15 and CAKI-1; Cell-Titer Glo assay for cell viability of cells treated with increasing concentrations of dBET6 (Panel M) or MZ1 (Panel N), tariquidar or the combination and bliss synergy scores determined; cells were treated with DMSO, tariquidar (0.1 μM), dBET6 (0.05 μM) (Panel M), MZ1 (0.1 μM) (Panel N) or the combination and colony formation assessed following 14-days of treatment; colony formation image representative of 3 independent assays. Data present in Panels C, G, J, M, and N are triplicate experiments SD; *p≤0.05 by student's t-test.

FIG. 5 shows re-purposing dual kinase/MDR1 inhibitors to overcome degrader resistance in cancer cells. Panels A and B show treatment of degrader-resistant cells with RAD001 or lapatinib reduces MDR1 drug efflux activity; A1847 parental, dBET6-R (Panel A) or Thal-R (Panel B) cells were treated with DMSO, 2 μM tariquidar, 2 μM RAD001, or 2 μM lapatinib and Rhodamine 123 efflux assessed. Panels C and D show degrader-resistant cells exhibit increased sensitivity towards RAD001; Cell-Titer Glo assay for cell viability of A1847 parental, dBET6-R (Panel C) or Thal-R (Panel D) cells treated with increasing concentrations of RAD001. Panels E and F show degrader-resistant cells exhibit increased sensitivity towards lapatinib; Cell-Titer Glo assay for cell viability of A1847 parental, dBET6-R (Panel C) or Thal-R (Panel D) cells treated with increasing concentrations of lapatinib. Panels G and H show treatment of degrader-resistant cells with RAD001 or lapatinib promotes degradation of PROTAC-targets; A1847 parental, dBET6-R (Panel G) or Thal-R (Panel H) cells treated with DMSO, RAD001 (2 μM) or lapatinib (2 μM) for 4 hours and proteins measured by western blot; blots are representative of 3 independent blots. Panels I and J show treatment of degrader-resistant cells with RAD001 or lapatinib induces apoptosis; A1847 parental, dBET6-R (Panel I) or Thal-R (Panel J) cells treated with DMSO, RAD001 (2 μM), lapatinib (2 μM) or tariquidar (2 μM) for 4 hours and proteins measured by western blot; blots are representative of 3 independent blots. Panel K shows treatment of MDR1-overexpressing cells with RAD001 or lapatinib reduces MDR1 drug efflux; DLD-1 cells were treated with DMSO, 2 μM tariquidar, 2 μM RAD001, or 2 μM lapatinib and Rhodamine 123 efflux assessed. Panels L and M show combined RAD001 or lapatinib-treatment improves PROTAC-mediated degradation of BRD4 in MDR1 overexpressing cells. DLD-1 cells were treated with increasing doses of dBET6 alone or in combination with RAD001 (2 μM) (Panel L) or lapatinib (2 μM) (Panel M) for 4 hours and BRD4 protein levels assessed by western blot; blots are representative of 3 independent blots. Panels N and O show KU-0063794 or Afatinib do not improve PROTAC-mediated degradation of BRD4 in MDR1 overexpressing cells; DLD-1 cells were treated with increasing doses of dBET6 alone or in combination with KU-0063794 (2 μM) (Panel N) or afatinib (2 μM) (Panel O) for 4 hours and BRD4 protein levels assessed by western blot; blots are representative of 3 independent blots. Panel P shows combining RAD001 or lapatinib but not KU-0063794 or Afatinib with BET degraders exhibits drug synergy in MDR1-overexpressing cells; DLD-1 cells were treated with DMSO, dBET6 (0.1 μM), lapatinib (2 μM), afatinib (2 μM), RAD001 (2 μM), KU-0063794 (2 μM) or in combination with dBET6 and colony formation assessed following 14-days of treatment; colony formation image representative of 3 independent assays. Panels Q and R show combined RAD001 or lapatinib-treatment improves PROTAC-mediated degradation of CDK9 in MDR1 overexpressing cells; DLD-1 cells were treated with increasing doses of Thal SNS 032 alone or in combination with RAD001 (2 μM) (Panel L) or lapatinib (2 μM) (Panel M) for 4 hours and CDK9 protein levels assessed by western blot; blots are representative of 3 independent blots. Panel S shows combining RAD001 or lapatinib with CDK9 degraders exhibits drug synergy in MDR1-overexpressing cells; DLD-1 cells were treated with DMSO, dBET6 (0.1 μM), lapatinib (2 μM), RAD001 (2 μM) or in combination with Thal SNS 032 and colony formation assessed following 14-days of treatment; colony formation image representative of 3 independent assays. Data present in Panels C, D, E, F, and K are triplicate experiments SD; *p<0.05 by student's t-test.

FIG. 6 shows combining MEK1/2 degraders with lapatinib synergize to kill MDR1-overexpressing K-ras mutant CRC cells and tumors. Panels A and B show MDR1 is overexpressed in the majority of K-ras mutant CRC cell lines. Panel A shows ABCB1 expression data was obtained from c-Bioportal. Panel B shows MDR1 protein levels across selected CRC cell lines was determined by western blot; blots are representative of 3 independent blots. Panels C and D show K-ras mutant CRC cells overexpressing MDR1 exhibit reduced sensitivity towards MEK1/2 degrader MS432; Panel C shows CRC cells were treated with escalating doses of MS432 for 5 days and cell viability assessed by CellTiter-Glo; GI50 values were determined in Prism software; Panel D shows CRC cells were treated with 1 μM of MS432 and colony formation assessed following 14 days of treatment; colony formation image representative of 3 independent assays. Panel E shows overexpression of MDR1 reduces PROTAC-mediated degradation efficiency in K-ras mutant CRC cells; CRC cells exhibiting different levels of MDR1 were treated with escalating doses of MS432 for 4 hours and MEK1/2 protein levels assessed by western blot; blots are representative of 3 independent blots. Panel F shows treatment of MDR1-overexpressing cells with tariquidar or lapatinib reduces MDR1 drug efflux; DLD-1 cells were treated with DMSO, 2 μM tariquidar, or 2 μM lapatinib and Rhodamine 123 efflux assessed. Panel G shows combined inhibition of MDR1 improves PROTAC-mediated degradation in MDR1 overexpressing cells; LS513 cells were treated with increasing doses of MS432 alone or in combination with tariquidar (0.1 μM) or increasing doses of MS432 alone or in combination with lapatinib (5 μM) for 24 hours and protein/phosphoprotein levels assessed by western blot; blots are representative of 3 independent blots. Panel H shows MEK inhibition upregulates ErbB receptor signaling and downstream AKT signaling in LS513 cells that can be blocked by lapatinib; LS513 cells were treated with DMSO, PD0325901 (0.01 mM), lapatinib (5 mM), or the combination for 48 hours and signaling assessed by western blot; blots are representative of 3 independent blots. Panels I and J show lapatinib but not tariquidar treatment blocks MEKi-induced ERBB3 reprogramming; LS513 cells were treated with DMSO, MS432 (1 μM), tariqudiar (0.1 μM) or the combination (Panel I) or DMSO, MS432 (1 μM), lapatinib (5 μM) or the combination (Panel J) and protein/phosphoproteins assessed by western blot; blots are representative of 3 independent blots. Panels K and L show combining lapatinib and MS432 exhibits drug synergy in MDR1-overexpressing K-ras mutant CRC cells; Cell-Titer Glo assay for cell viability of LS513 cells treated with increasing concentrations of MS432, lapatinib or the combination of lapatinib and MS432 (Panel H); Bliss synergy scores determined. LS513 cells were treated with DMSO, lapatinib (5 μM), MS432 (1 μM) or the combination and colony formation assessed following 14-days of treatment (Panel I); colony formation image representative of 3 independent assays. Panel M shows lapatinib in combination with MS432 enhances growth inhibition in MDR1-overexpressing K-ras mutant CRC cell lines; CRC cell lines were treated with DMSO, lapatinib (5 μM), MS432 (1 μM), or the combination and colony formation assessed following 14-days of treatment; colony formation image representative of 3 independent assays. Panels N and O show co-treatment with MS934 and lapatinib MDR1 improves PROTAC-mediated degradation in MDR1 overexpressing cells; LS513 cells were treated with increasing doses of MS934 alone or in combination with lapatinib (5 μM) for 24 hours and protein/phosphoprotein levels assessed by western blot; blots are representative of 3 independent blots. Panels P and Q show MEK degraders in combination with lapatinib reduce tumor growth in vivo; LS513 cells were grown as xenografts in nude mice and treated with vehicle, 50 mg/kg MS934, 100 mg/kg lapatinib, or the combination of MS934 and lapatinib and tumor volume determined (Panel M); body weight of animals was determined to evaluate potential toxicities of drug treatments (Panel N); N=5 per treatment group, Error bar+SEM. Data present in Panels C, F, K, and L are triplicate experiments SD; *p≤0.05 by student's t-test.

FIG. 7 shows 1Lapatinib-treatment improves KRASG12C degrader therapies in MDR1-overexpressing CRC cell lines. Panels A and B show MDR1-overexpressing KRASG12C mutant CRC cell lines are resistant to LC-2 but sensitive to K-ras inhibitors; SW1463 or SW837 cell lines were treated with DMSO, LC-2 (1 μM) or MRTX849 (1 μM) and colony formation assessed following 14-days of treatment; colony formation image representative of 3 independent assays. Panels C and D show lapatinib in combination with LC-2 but not tariquidar inhibits KRASG12C effector signaling; SW1463 cells were treated with DMSO, MS432 (1 μM), lapatinib (5 μM), tariquidar (0.01 μM) or the combination of MS432/lapatinib or MS432/tariquidar for 48 hours and protein/phosphoprotein levels assessed by western blot; blots are representative of 3 independent blots. Panel E shows combination therapies involving LC-2 and lapatinib block KRASG12C effector signaling; SW837 cells were treated with DMSO, MS432 (1 μM), lapatinib (5 μM) or the combination of MS432/lapatinib for 48 hours and protein/phosphoprotein levels assessed by western blot. Panels F and G show combining lapatinib and LC-2 exhibits drug synergy in MDR1-overexpressing KRASG12C CRC cells; Cell-Titer Glo assay for cell viability of SW1463 (Panel G) or SW837 (Panel H) cells treated with increasing concentrations of LC-2, lapatinib or the combination and bliss synergy scores determined. Panels H and I show combining lapatinib with LC-2 exhibits durable growth inhibition in MDR1-overexpressing KRASG12C CRC cells; SW1463 (Panel I) or SW837 (Panel J) cells were treated with DMSO, LC-2 (1 μM), lapatinib (5 μM), tariquidar (0.01 μM) or the combination of MS432/lapatinib or MS432/tariquidar and colony formation assessed following 14-days of treatment; colony formation image representative of 3 independent assays. Panel J shows rationale for combining lapatinib with MEK1/2 or KRASG12C degraders in MDR1-overexpressing CRC cell lines; simultaneous blockade of MDR1 and ErbB receptor signaling overcomes degrader resistance as well as ErbB receptor kinome reprogramming resulting in sustained inhibition of Kras effector signaling. Data present in Panels F and G, are triplicate experiments SD; *p≤0.05 by student's t-test.

FIG. 8 shows proteomics characterization of degrader-resistant cancer cell lines. Panel A shows A1847 cells acquire resistance to MZ1; parental or MZ1-resistant cells were treated with escalating doses of MZ1 for 5 days and cell viability was assessed by CellTiter-Glo; MZ1-R treated cell viabilities normalized to DMSO treated degrader-R cells. Panel B shows escalating doses of MZ1 less effective at inducing degradation of BET proteins in MZ1-R cells; A1847 parental or MZ1-R cells were treated with escalating doses of MZ1 (0, 0.039, 0.156, 0.625, 2.5 or 10 μM) for 24 hours and degrader targets and downstream signaling determined by western blot; blots are representative of 3 independent blots. Panel C shows Volcano plot depicts proteins elevated or reduced in MZ1-R relative to parental A1847 cells; differences in protein log 2 LFQ intensities amongst degrader-resistant and parental cells were determined by paired t-test Benjamini-Hochberg adjusted P values at FDR of <0.05 using Perseus software. Panel D shows the top 10 upregulated proteins in MZ1-R relative to parental A1847 cells. Panel E shows a bar graph that depicts ABCB1 log 2 LFQ values comparing MZ1-R relative to parental A1847 cells; differences in ABCB1 log 2 LFQ intensities amongst MZ1-R and parental cells were determined by paired t-test Benjamini-Hochberg adjusted P values at FDR of <0.05 using Perseus software. Panel F shows SUM159 cells acquire resistance to MZ1; parental or MZ1-resistant cells were treated with escalating doses of MZ1 for 5 days and cell viability assessed by CellTiter-Glo; MZ1-R treated cell viabilities normalized to DMSO treated degrader-R cells. Panel G shows escalating doses of MZ1 less effective at inducing degradation of BET proteins in MZ1-R cells; SUM159 parental or MZ1-R cells were treated with escalating doses of MZ1 (0, 0.039, 0.156, 0.625, 2.5 or 10 μM) for 24 hours and degrader targets and downstream signaling determined by western blot; blots are representative of 3 independent blots. Panel H shows Volcano plot that depicts proteins elevated or reduced in MZ1-R relative to parental SUM159 cells; differences in protein log 2 LFQ intensities amongst degrader-resistant and parental cells were determined by paired t-test Benjamini-Hochberg adjusted P values at FDR of <0.05 using Perseus software. Panel I shows the top 10 upregulated proteins in MZ1-R relative to parental SUM159 cells. Panel J shows a bar graph that depicts ABCB1 log 2 LFQ values comparing MZ1-R relative to parental SUM159 cells; differences in ABCB1 log 2 LFQ intensities amongst MZ1-R and parental cells were determined by paired t-test Benjamini-Hochberg adjusted P values at FDR of <0.05 using Perseus software. Data present in Panels A and F are triplicate experiments SD; *p≤0.05 by student's t-test.

FIG. 9 shows chronic exposure to degraders induces MDR1 expression and drug efflux activity. Panel A shows ABCB1 mRNA levels are upregulated in MZ1-R cells relative to parental A1847 cells as determined by qRT-PCR. Panel B shows MDR1 protein levels are upregulated in MZ1-R cell lines relative to parental cells as determined by immunoblot; blots are representative of 3 independent blots. Data present in Panel A are triplicate experiments SD; *p≤0.05 by student's t-test.

FIG. 10 shows blockade of MDR1 activity re-sensitizes degrader-resistant cells to PROTACs. Panel A shows degrader-resistant cells also display resistance to MDR1-substrate paclitaxel; A1847 parental, dBET6-R or Thal-R cells were treated with doses of MZ1 for 5 days and cell viability assessed by CellTiter-Glo. Panels B and C show degrader-resistant cells cross-resistant to other PROTACs targeting different proteins; A1847 Parental or dBET6-R cells were treated with DMSO or (1 μM) Thal SNS 032 (Panel B) or (1 μM) FAK-degrader-1 (Panel C) for 24 hours and proteins assessed by western blot. Panel D shows chronic exposure to BET inhibitor JQ1 does not sensitize A1847 cells to MDR1 inhibition; A1847 parental, dBET6-R or JQ1-R cells were treated with doses of MZ1 for 5 days and cell viability assessed by CellTiter-Glo. Panels E and F show OVCAR3 and HCT116 cells acquire resistance to MZ1; parental or OVCAR3 (Panel E) or HCT116 (Panel F) MZ1-resistant cells were treated with escalating doses of MZ1 for 5 days and cell viability assessed by CellTiter-Glo; MZ1-R treated cell viabilities normalized to DMSO treated degrader-R cells. Panels G and H show MZ1-resistant OVCAR3 and HCT116 cells exhibit increased sensitivity to MDR1 inhibitors; Cell-Titer Glo assay for cell viability of parental, MZ1-R OVCAR3 (Panel G) or MZ1-R HCT116 (Panel H) treated with increasing concentrations of MDR1 inhibitor tariquidar. Panel I shows MZ1 chronically exposed MOLT4 cells retain sensitivity towards MZ1 therapies; MOLT4 parental or MZ1-R cells were treated with doses of MZ1 for 5 days and cell viability assessed by CellTiter-Glo. Data present in Panels A, D, E, F, G, H, and I are triplicate experiments SD; *p<0.05 by student's t-test.

FIG. 11 shows overexpression of MDR1 conveys intrinsic resistance to degrader therapies in cancer cells. Panel A shows frequency of ABCB1 mRNA overexpression across cancer cell line panel; expression data was queried from cBioPortal for cancer genomics using Z-scores values of >2-fold-change for ABCB1 mRNA levels (Gao et al., Science Signaling, 2013, 6, pl1-pl1). Panel B shows MDR1 protein expression across tumor samples by immunohistochemistry; MDR1 protein data was queried from the human protein atlas; MDR1 expression was determined by immunohistochemistry using MDR1 antibody CAB001716 (Uhlen et al., Science, 2015, 347, 1260419). Panels C and D show cancer cells overexpressing MDR1 exhibit reduced sensitivity towards dBET6 or MZ1; cancer cells were treated with escalating doses of dBET6 (Panel C) or MZ1 (Panel D) for 5 days and cell viability assessed by CellTiter-Glo; GI50 values were determined in Prism software. Panels E and F show inhibition of MDR1 sensitizes CRC cell line to other PROTACs; DLD-1 cells were treated with DMSO or (1 μM) FAK-degrader-1 (Panel E) or (1 μM) MS432 (Panel F) for 24 hours and proteins assessed by western blot. Panels G and H show combining tariquidar with CDK9 degraders enhances growth inhibition of MDR1-overexpressing cell lines; Cell-Titer Glo assay for cell viability of HCT-15 (Panel G) or CAKI-1 (Panel H) cells treated with increasing concentrations of Thal SNS 032, tariquidar or the combination and bliss synergy scores determined; cells were treated with DMSO, tariquidar (0.1 μM), Thal SNS 032 (5 μM) or the combination and colony formation assessed following 14-days of treatment; colony formation image representative of 3 independent assays. Data present in Panels C, D, G, and H are triplicate experiments SD; *p≤0.05 by student's t-test.

FIG. 12 shows re-purposing dual kinase/MDR1 inhibitors to overcome degrader resistance in cancer cells. Panels A and B show MZ1-resistant cells exhibit increased sensitivity towards RAD001; Cell-Titer Glo assay for cell viability of A1847 parental or MZ1-R (Panel A) or SUM159 parental or MZ1-R (Panel B) cells treated with increasing concentrations of RAD001. Panels C and D show MZ1-resistant cells exhibit increased sensitivity towards lapatinib; Cell-Titer Glo assay for cell viability of A1847 parental or MZ1-R (Panel A) or SUM159 parental or MZ1-R (Panel B) cells treated with increasing concentrations of lapatinib. Panels E and F show treatment of MZ1-resistant cells with RAD001 or lapatinib promotes degradation of PROTAC-targets; A1847 parental, or MZ1-R cells treated with DMSO, RAD001 (2 μM) (Panel E) or lapatinib (2 μM) (Panel F) for 4 hours and proteins measured by western blot. Panel G shows inhibitors Afatinib and KU-0063794 do not block MDR1 activity in degrader-resistant cells; treatment of A1847 Thal-R cells with Afatinib or KU-0063794 does not reduce MDR1 drug efflux activity; A1847 Thal-R cells were treated with DMSO, 2 μM tariquidar, 2 μM RAD001, 2 μM lapatinib, 2 μM Afatinib or 2 μM KU-0063794 and Rhodamine 123 efflux assessed. Data present in Panels A, B, C, D, G are triplicate experiments SD; *p≤0.05 by student's t-test.

FIG. 13 shows combining MEK1/2 degraders with lapatinib synergize to kill MDR1-overexpressing K-ras mutant CRC cells. Panel A shows CRC cell lines explored in MS432 studies exhibit sensitivity towards MEK inhibition; drug sensitivity profile of CRC cell lines to trametinib treatment; the bar graph depicts AUC from trametinib dose response studies; AUC data was queried from DepMap databases (Barretina et al., Nature, 2012, 483, 603-607). Panel B shows combining lapatinib with MEK inhibitors enhances growth inhibition of LS513 CRC cells; Cell-Titer Glo assay for cell viability of LS513 cells treated with increasing concentrations of lapatinib, PD0325901 or the combination and bliss synergy scores determined. Panel C shows combining lapatinib with MEK degrader MS934 enhances growth inhibition of LS513 CRC cells; Cell-Titer Glo assay for cell viability of LS513 cells treated with increasing concentrations of lapatinib, MS934 or the combination and bliss synergy scores determined. Data present in Panels B and C are triplicate experiments SD; *p<0.05 by student's t-test.

FIG. 14 shows lapatinib-treatment improves KRASG12C degrader therapies in MDR1-overexpressing CRC cell lines. Panels A and B show combining tariquidar with KRASG12C degraders display less drug synergy than when combined with lapatinib in CRC cells; Cell-Titer Glo assay for cell viability of SW1463 (Panel A) or SW837 (Panel B) cells treated with increasing concentrations of lapatinib, LC-2 or the combination and bliss synergy scores determined. Data present in Panels A and B are triplicate experiments SD; *p<0.05 by student's t-test.

### DESCRIPTION OF EMBODIMENTS

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the terms “augment” and “augmenting”, for therapeutic purposes, generally refers to an improvement in the pharmacodynamic effect (referred to as the efficacy) of a therapeutic agent. Thus, the term “augment” refers to the ability of the kinase inhibitors, KRAS inhibitors, or autophagy inhibitors to raise the efficacy of PROTACs leading to the killing of a greater number of cancer cells over the same unit of time (e.g., 24, 48, or 72 hour period) when the kinase inhibitors, KRAS inhibitors, or autophagy inhibitors are administered prior to, along with, or after the PROTACs as compared to the PROTACs alone.

As used herein, the terms “cancer” and “cancerous” refer to or describe the physiological condition in mammals that is typically characterized by unregulated cell growth/proliferation. A “tumor” comprises one or more cancerous cells. Examples of cancer are provided elsewhere herein.

As used herein, the terms “co-administration” and “co-administering” and “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, one or more of the present compounds described herein, are coadministered in combination with at least one additional bioactive agent, especially including an anticancer agent. In particularly preferred aspects, the co-administration of compounds results in synergistic activity and/or therapy, including anticancer activity.

As used herein, all transitional phrases such as “comprising,” “including,” “carrying,” “having,” “containing,” “involving,” “holding,” “composed of,” and the like are to be understood to be open-ended, i.e., to mean including but not limited to. The term “comprising” may be replaced with “consisting” or “consisting essentially of” in particular embodiments as desired.

As used herein, the term “concurrently” means that a drug that is administered with one or more other drugs is administered during the same treatment cycle, on the same day of treatment as the one or more other drugs, and, optionally, at the same time as the one or more other drugs. For instance, for cancer therapies given every 3 weeks, the concurrently administered drugs are each administered on day-1 of a 3-week cycle.

As used herein, the term “disease free survival (DFS)” refers to the patient remaining alive, without return of the cancer, for a defined period of time such as about 1 year, about 2 years, about 3 years, about 4 years, about 5 years, about 10 years, etc., from initiation of treatment or from initial diagnosis. In one aspect of the subject matter described herein, DFS is analyzed according to the intent-to-treat principle, i.e., patients are evaluated on the basis of their assigned therapy. The events used in the analysis of DFS can include local, regional and distant recurrence of cancer, occurrence of secondary cancer, and death from any cause in patients without a prior event (e.g., breast cancer recurrence or second primary cancer).

As used herein, the term “effective” is used to describe an amount of a compound, composition or component which, when used within the context of its intended use, effects an intended result. The term effective subsumes all other effective amount or effective concentration terms, which are otherwise described or used in the present application.

As used herein, the term “effective amount” of an agent, e.g., a pharmaceutical formulation, refers to an amount effective, at dosages and for periods of time necessary, to achieve the desired therapeutic or prophylactic result. For example, an effective amount of the drug for treating cancer may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. The effective amount may extend progression free survival (e.g. as measured by Response Evaluation Criteria for Solid Tumors, RECIST, or CA-125 changes), result in an objective response (including a partial response, PR, or complete response, CR), increase overall survival time, and/or improve one or more symptoms of cancer (e.g. as assessed by FOSI).

As used herein, the term “extending survival” means increasing DFS and/or OS in a treated patient relative to an untreated patient, or relative to a control treatment protocol. Survival is monitored for at least about six months, or at least about 1 year, or at least about 2 years, or at least about 3 years, or at least about 4 years, or at least about 5 years, or at least about 10 years, etc., following the initiation of treatment or following the initial diagnosis.

As used herein, the terms “linker”, “linker unit”, or “link” mean a chemical moiety comprising a chain of atoms that covalently attaches a PROTAC moiety to an antibody, or a component of a PROTAC to another component of the PROTAC. In some embodiments, a linker is a divalent radical.

As used herein, the term “optionally” means that the subsequently described event(s) may or may not occur, and includes both event(s) that occur and event(s) that do not occur.

As used herein, the phrase “optionally substituted”, “substituted” or variations thereof denote an optional substitution, including multiple degrees of substitution, with one or more substituent group, for example, one, two or three. The phrase should not be interpreted as duplicative of the substitutions herein described and depicted.

As used herein, the term “overall survival” refers to the patient remaining alive for a defined period of time, such as about 1 year, about 2 years, about 3 years, about 4 years, about 5 years, about 10 years, etc., from initiation of treatment or from initial diagnosis.

As used herein, the term “pharmaceutical formulation” refers to a preparation which is in such form as to permit the biological activity of an active ingredient contained therein to be effective, and which contains no additional components which are unacceptably toxic to a subject to which the formulation would be administered.

As used herein, the term “pharmaceutically acceptable excipient” refers to an ingredient in a pharmaceutical formulation, other than an active ingredient, which is nontoxic to a subject. A pharmaceutically acceptable excipient includes, but is not limited to, a buffer, carrier, stabilizer, or preservative.

As used herein, the term “pharmaceutically acceptable salt,” as used herein, refers to pharmaceutically acceptable organic or inorganic salts of a molecule. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion. Other salts, which are not pharmaceutically acceptable, may be useful in the preparation of compounds of described herein and these should be considered to form a further aspect of the subject matter. These salts, such as oxalic or trifluoroacetate, while not in themselves pharmaceutically acceptable, may be useful in the preparation of salts useful as intermediates in obtaining the compounds described herein and their pharmaceutically acceptable salts.

As used herein, the term “progression-Free Survival” (PFS) is the time from the first day of treatment to documented disease progression (including isolated CNS progression) or death from any cause on study, whichever occurs first.

As used herein, the term “PROTAC” refers to proteolysis-targeting chimera molecules having generally three components, an E3 ubiquitin ligase binding group (E3LB), a linker L, and a protein binding group (PB).

As used herein, the terms “subject” and “patient” are used interchangeably. A subject may include any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human.

As used herein, the term “survival” refers to the patient remaining alive, and includes disease free survival (DFS), progression free survival (PFS) and overall survival (OS). Survival can be estimated by the Kaplan-Meier method, and any differences in survival are computed using the stratified log-rank test.

As used herein, the term “therapeutically effective amount” means any amount which, as compared to a corresponding subject who has not received such amount, results in treatment of a disease, disorder, or side effect, or a decrease in the rate of advancement of a disease or disorder. The term also includes within its scope amounts effective to enhance normal physiological function. For use in therapy, therapeutically effective amounts of a PAC, as well as salts thereof, may be administered as the raw chemical. Additionally, the active ingredient may be presented as a pharmaceutical composition.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, compositions described herein are used to delay development of a disease or to slow the progression of a disease. In some embodiments, compositions described herein are used to increase survival of a patient having a disease. In some embodiments, compositions described herein are used to increase or extend survival of a patient having a disease.

As used herein, the term “ubiquitin ligase” refers to a family of proteins that facilitate the transfer of ubiquitin to a specific substrate protein, targeting the substrate protein for degradation. For example, cereblon is an E3 ubiquitin ligase protein that alone or in combination with an E2 ubiquitin-conjugating enzyme causes the attachment of ubiquitin to a lysine on a target protein, and subsequently targets the specific protein substrates for degradation by the proteasome. Thus, E3 ubiquitin ligase alone or in complex with an E2 ubiquitin conjugating enzyme is responsible for the transfer of ubiquitin to targeted proteins. In general, the ubiquitin ligase is involved in polyubiquitination such that a second ubiquitin is attached to the first; a third is attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins are not targeted to the proteasome for degradation, but may instead be altered in their cellular location or function, for example, via binding other proteins that have domains capable of binding ubiquitin. Further complicating matters, different lysines on ubiquitin can be targeted by an E3 to make chains. The most common lysine is Lys48 on the ubiquitin chain. This is the lysine used to make polyubiquitin, which is recognized by the proteasome.

It should also be understood that, in certain methods described herein that include more than one step or act, the order of the steps or acts of the method is not necessarily limited to the order in which the steps or acts of the method are recited unless the context indicates otherwise.

It should be appreciated that particular features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

It should be understood that stereoisomers (including diastereomers and enantiomers) of the compounds described herein, as well as mixtures thereof, are within the scope of the present disclosure. By way of non-limiting example, the mixture may be a racemate or the mixture may comprise unequal proportions of one particular stereoisomer over the other. Additionally, the compounds can be provided as a substantially pure stereoisomers. Diastereomers include, for example, cis-trans isomers, E-Z isomers, conformers, and rotamers. Methods of preparation of stereoisomers are known in the art, such as by resolution of racemic mixtures or by stereoselective synthesis. Many geometric isomers of olefins, C═N double bonds, and the like can also be present in the compounds described herein, and all such stable isomers are contemplated herein. Cis and trans geometric isomers of the compounds are also included herein and can be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated.

Appropriate compounds described herein may also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

The compounds described herein also include hydrates and solvates, as well as anhydrous and non-solvated forms.

The compounds described herein also include derivatives referred to as prodrugs, which can be prepared by modifying functional groups present in the compounds in such a way that the modifications are cleaved, either in routine manipulation or in vivo, to the parent compounds. Examples of prodrugs include compounds as described herein that contain one or more molecular moieties appended to a hydroxyl, amino, sulfhydryl, or carboxyl group of the compound, and that when administered to a patient, cleaves in vivo to form the free hydroxyl, amino, sulfhydryl, or carboxyl group, respectively. Examples of prodrugs include, but are not limited to, acetate, formate and benzoate derivatives of alcohol and amine functional groups in the compounds described herein. Preparation and use of prodrugs is discussed in T. Higuchi et al., “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

The present disclosure provides pharmaceutical compositions comprising: a) one or more proteolysis targeting chimera (PROTAC) therapeutic agents; and b) one or more kinase inhibitors, one or more KRAS inhibitors, or one or more autophagy inhibitors, or any combination thereof. In some embodiments, when the pharmaceutical composition comprises one or more kinase inhibitors, one or more KRAS inhibitors, or one or more autophagy inhibitors, or any combination thereof, the PROTAC in the pharmaceutical composition is not a bromodomain and extra terminal domain (BET) PROTAC (BET-PROTAC) or a cyclin-dependent kinase 9 (CDK9) PROTAC (CDK9-PROTAC). The present disclosure is based on a surprising and unexpected discovery that resistance of cancer to PROTAC therapeutic agents can be overcome by inhibiting one or more cell signaling pathways including RTK signaling pathway, mTOR signaling pathway, CDK7 signaling pathway, KRAS signaling pathway or an autophagy signaling pathway.

The present disclosure also provides pharmaceutical compositions comprising: a) a PROTAC therapeutic agent; and b) one or more kinase inhibitors, one or more KRAS inhibitors, one or more autophagy inhibitors, or one or more ABC transporter inhibitors, or any combination thereof. In some embodiments, the pharmaceutical compositions comprise: a) a PROTAC therapeutic agent; and b) one or more ABC transporter inhibitors. In some embodiments, the pharmaceutical compositions comprise: a) a PROTAC therapeutic agent; and b) one or more dual ABC transporter/kinase inhibitors.

A drug combination therapy has been identified herein that enhances PROTAC therapies improving degradation of targets and therapeutic responses. Dual inhibitors that block the function or reduce the levels of ABCB1 and inhibit ErbB receptors, MTOR/AKT or other kinase inhibitor function will sensitize cells to PROTAC therapies improving drug efficacy and therapeutic responses. Furthermore, the compositions described herein can block or overcome acquired resistance to PROTAC therapies. Patients with low or overexpressed ABCB1 will benefit from combining PROTAC therapies with dual ABCB1 and kinase inhibitors. This includes patients that have acquired resistance to chemotherapy agents by multidrug pump activation.

The compositions described herein can be used to treat patients with disease in which PROTAC therapies have been developed. The compositions described herein are rationally predicted combination therapy for PROTAC therapies based on proteomics characterization of cells that developed resistance or exhibited intrinsic resistance to the PROTAC therapies. Patients that have cancer or other diseases can be treated with the invented combination therapy and are predicted to have a more durable therapeutic response than if patients were treated solely with the PROTAC therapy. Moreover, patients exhibiting low or elevated ABCB1 protein levels in tumor or tissue samples will represent a target population that will benefit from the compositions described herein. Specific types of diseases that have high ABCB1 levels, are liver, colorectal, and kidney, as well as other cancers, which would be a target population that would benefit strongly from the compositions described herein. Moreover, patients with low levels of ABCB1 protein will also benefit from the compositions described herein, as continuous treatment of tissues with PROTACs causes an increase in expression of ABCB1. By combining the drug pump inhibitor, the dose of the PROTAC can be significantly reduced 100-fold and achieve similar anti-growth and death inducing properties as “typical” doses of the single agent PROTAC current used to degrade target protein.

The compositions described herein solve the problem of intrinsic and acquired drug resistance to PROTAC therapies by providing a drug therapy that can kill cancer cells resistant to PROTAC drugs, as well as prevent the ability of cancer cells to acquire resistance to PROTAC therapies. Moreover, resistance to PROTACs targeting proteins associated with receptor tyrosine kinase signaling, RAS, PI3K or epigenetic signaling in cancer cells involves upregulation of ErbB receptors (EGFR, ERBB2, ERBB3, ERBB3), MTOR/AKT or other survival kinase activities. The compositions described herein provide a combination therapy that can overcome resistance to PROTACs, as well as the resistance associated with on-target engagement of the protein targeted by the PROTAC.

As shown herein, cancer cells can acquire resistance to any PROTAC therapy by upregulating ABCB1 drug efflux activity. Cells that have low to non-detectable ABCB1 protein levels can increase ABCB1 protein levels by many folds following chronic exposure to PROTAC therapies making them resistant to PROTAC therapies. Resistance to therapies targeting proteins associated with receptor tyrosine kinase signaling, RAS, PI3K or epigenetic signaling in cancer cells involves upregulation of ErbB receptors (EGFR, ERBB2, ERBB3, ERBB3), MTOR/AKT or other survival kinase activities. The present disclosure combines an inhibitor targeting the resistance kinases associated with degradation of proteins involved in receptor tyrosine kinase signaling, RAS, PI3K or epigenetic signaling, while simultaneously blocking the efflux of PROTACs from cells by ABCB1.

It has been demonstrated herein that combining PROTACs targeting MEK1/2 (MS934) with the dual ABCB1/ErbB receptor inhibitor lapatinib improves the anti-tumor properties in vivo using xenografts of KRAS mutant colorectal cancer cells.

Proteolysis targeting chimeras are described, for example, in U. S. Patent Application Publication No. US 2019/0175612, U.S. Pat. No. 7,208,157, and PCT Publications No. WO2013/106643 and WO2013/106646. PROTACs have a general structure E3LB-L-PB; wherein, E3LB is an E3 ligase binding group covalently bound to L; L is a linker covalently bound to E3LB and PB; PB is a protein binding group covalently bound to L.

E3 ubiquitin ligases (of which over 600 are known in humans) confer substrate specificity for ubiquitination. There are known ligands which bind to these ligases. As described herein, an E3 ubiquitin ligase binding group is a peptide or small molecule that can bind an E3 ubiquitin ligase. Specific E3 ubiquitin ligases include: von Hippel-Lindau (VHL); cereblon, XIAP, E3A; MDM2; Anaphase-promoting complex (APC); UBR5 (EDD1); SOCS/BC-box/eloBC/CUL5/RING; LNXp80; CBX4; CBLL1; HACE1; HECTD1; HECTD2; HECTD3; HECW1; HECW2; HERC1; HERC2; HERC3; HERC4; HUWE1; ITCH; NEDD4; NEDD4L; PPIL2; PRPF19; PIAS1; PIAS2; PIAS3; PIAS4; RANBP2; RNF4; RBX1; SMURF1; SMURF2; STUB1; TOPOR5; TRIP12; UBE3A; UBE3B; UBE3C; UBE4A; UBE4B; UBOX5; UBR5; WWP1; WWP2; Parkin; A20/TNFAIP3; AMFR/gp78; ARA54; beta-TrCP1/BTRC; BRCA1; CBL; CHIP/STUB1; E6; E6AP/UBE3A; F-box protein 15/FBXO15; FBXW7/Cdc4; GRAII/RNF128; HOIP/RNF31; cIAP-1/HIAP-2; cIAP-2/HIAP-1; cIAP (pan); ITCH/AIP4; KAP1; MARCH8; Mind Bomb 1/MIB1; Mind Bomb 2/MIB2; MuRF1/TRIM63; NDFIP1; NEDD4; NleL; Parkin; RNF2; RNF4; RNF8; RNF168; RNF43; SART1; Skp2; SMURF2; TRAF-1; TRAF-2; TRAF-3; TRAF-4; TRAF-5; TRAF-6; TRIM5; TRIM21; TRIM32; UBR5; and ZNRF3.

In some embodiments, E3 ubiquitin ligase is von Hippel-Lindau (VHL) tumor suppressor, the substrate recognition subunit of the E3 ligase complex VCB, which also consists of elongins B and C, Cul2 and Rbx1. The primary substrate of VHL is Hypoxia Inducible Factor 1α (HIF-1α), a transcription factor that upregulates genes such as the pro-angiogenic growth factor VEGF and the red blood cell inducing cytokine erythropoietin in response to low oxygen levels. Compounds that bind VHL may be hydroxyproline compounds such as those disclosed in WO2013/106643, and other compounds described in US2016/0045607, WO2014187777, US20140356322, and U.S. Pat. No. 9,249.

In some embodiments, E3 ubiquitin ligase is X-linked inhibitor of apoptosis (XIAP). XIAP is a protein that stops apoptotic cell death. Deregulation of XIAP has been associated with cancer, neurodegenerative disorders and autoimmunity. In the development of lung cancer, the overexpression of XIAP inhibits caspases. In developing prostate cancer, XIAP is one of four IAPs overexpressed in the prostatic epithelium. Mutations in the XIAP gene can result in a severe and rare type of inflammatory bowel disease. Defects in the XIAP gene can also result in an extremely rare condition called X-linked lymphoproliferative disease. Degradation of XIAP can enhance apoptosis by preventing XIAP from binding to caspases. This allows normal caspase activity to proceed.

Examples of small molecular binding compounds for XIAP include compounds disclosed in U.S. Pat. No. 9,096,544; WO 2015187998; WO 2015071393; U.S. Pat. Nos. 9,278,978; 9,249,151; US 20160024055; US 20150307499; US 20140135270; US 20150284427; US 20150259359; US 20150266879; US 20150246882; US 20150252072; US 20150225449; U.S. Pat. No. 8,883,771, J. Med. Chem., 2015, 58(16) 6574-6588 and Small-molecule Pan-IAP Antagonists: A Patent Review (2010) Expert Opin Ther Pat; 20: 251-67 (Flygare & Fairbrother).

In some embodiments, E3 ubiquitin ligase is MDM2. Examples of small molecular binding compounds for MDM2 include the “nutlin” compounds, e.g., nutlin 3a and nutlin 3.

Thalidomide, lenalidomide, pomalidomide and analogs thereof are known to bind to cereblon. The crystal structure of cereblon (CRBN) with thalidomide and derivative compounds are described in US2015/0374678. Other small molecule compounds that bind to cereblon are also known, e.g., the compounds disclosed as an in US2016/0058872 and US2015/0291562. Further, phthalimide conjugation with binders, such as antagonists, of BET bromodomains can provide PROTACs with highly-selective cereblon-dependent BET protein degradation. Winter et al., Science, Jun. 19, 2015, 1376. Such PROTACs can be conjugated to an antibody as described herein to form a PAC. Additional E3 ligase binding groups are described, for example, in the U. S. Patent Application Publication No. 2019/0175612.

The PB component is a group which binds to a target protein intended to be degraded. The term “protein” includes oligopeptides and polypeptide sequences of sufficient length that they can bind to a PB group. Any protein in a eukaryotic system or a microbial system, including a virus, bacteria or fungus, as otherwise described herein, are targets for ubiquitination mediated by the compounds described herein.

PB groups include, for example, any moiety which binds to a protein specifically (binds to a target protein) and includes the following non-limiting examples of small molecule target protein moieties: Hsp90 inhibitors, kinase inhibitors (such as CDK9 inhibitors), MDM2 inhibitors, compounds targeting Human BET Bromodomain-containing proteins, HDAC inhibitors, human lysine methyltransferase inhibitors, angiogenesis inhibitors, immunosuppressive compounds, and compounds targeting the aryl hydrocarbon receptor (AHR), among numerous others.

The E3LB and PB groups of PROTACs as described herein can be connected with linker (L). In certain embodiments, the linker group L is a group comprising one or more covalently connected structural units of A (e.g., −A₁ . . . A_{q-}), wherein A₁ is a group coupled to at least one of a E3LB, a PB, or a combination thereof. In certain embodiments, A₁ links a E3LB, a PB, or a combination thereof directly to another E3LB, PB, or combination thereof. In other embodiments, A₁ links a EL3B, a PB, or a combination thereof indirectly to another E3LB, PB, or combination thereof through A_{q}.

In some embodiments, q is an integer greater than or equal to 0. In certain embodiments, q is an integer greater than or equal to 1.

In some embodiments, e.g., where q is greater than 2, A_{q} is a group which is connected to an E3LB moiety, and A₁ and A_{q} are connected via structural units of A (number of such structural units of A: q−2). In some embodiments, e.g., where q is 2, A_{q} is a group which is connected to A₁ and to an E3LB moiety. In some embodiments, e.g., where q is 1, the structure of the linker group L-A₁-, and A₁ is a group which is connected to an E3LB moiety and a PB moiety.

In additional embodiments, q is an integer from 1 to 100, 1 to 90, 1 to 80, 1 to 70, 1 to 60, 1 to 50, 1 to 40, 1 to 30, 1 to 20, or 1 to 10.

In some embodiments, the linker group is an optionally substituted (poly)ethyleneglycol having from 1 to about 100 ethylene glycol units, from 1 to about 50 ethylene glycol units, from 1 to about 25 ethylene glycol units, from 1 to about 10 ethylene glycol units, from 1 to about 8 ethylene glycol units, and from 1 to about 6 ethylene glycol units, from 2 to 4 ethylene glycol units, or optionally substituted alkyl groups interdispersed with optionally substituted, O, N, S, P or Si atoms. In some embodiments, the linker is substituted with an aryl, phenyl, benzyl, alkyl, alkylene, or heterocycle group. In some embodiments, the linker may be asymmetric or symmetrical.

In any of the embodiments of the compounds described herein, the linker group may be any suitable moiety as described herein. In some embodiments, the linker is a substituted or unsubstituted polyethylene glycol group ranging in size from about 1 to about 12 ethylene glycol units, from 1 to about 10 ethylene glycol units, from 2 to about 6 ethylene glycol units, from 2 to about 5 ethylene glycol units, or from 2 to 4 ethylene glycol units.

Although the E3LB group and PB group may be covalently linked to the linker group through any group which is appropriate and stable to the chemistry of the linker. The linker can be independently covalently bonded to the E3LB group and the PB group preferably through an amide, ester, thioester, keto group, carbamate (urethane), carbon or ether, each of which groups may be inserted anywhere on the E3LB group and PB group to provide maximum binding of the E3LB group on the ubiquitin ligase and the PB group on the target protein to be degraded. In some embodiments where the PB group is an E3LB group, the target protein for degradation may be the ubiquitin ligase itself. In some embodiments, the linker may be linked to an optionally substituted alkyl, alkylene, alkene or alkyne group, an aryl group or a heterocyclic group on the E3LB and/or PB groups. It is noted that an E3LB group or a PB group may need to be derivatized to make a chemical functional group that is reactive with a chemical functional group on the linker. Alternately, the linker may need to be derivatized to include a chemical functional group that can react with a functional group found on E3LB and/or PB.

Although the E3LB group and PB group may be covalently linked to the linker group through any group which is appropriate and stable to the chemistry of the linker, in preferred aspects, the linker is independently covalently bonded to the E3LB group and the PB group through an amide, ester, thioester, keto group, carbamate (urethane) or ether, each of which groups may be inserted anywhere on the E3LB group and PB group to allow binding of the E3LB group to the ubiquitin ligase and the PB group to the target protein to be degraded. In other words, as shown herein, the linker can be designed and connected to E3LB and PB to minimize, eliminate, or neutralize any impact its presence might have on the binding of E3LB and PB to their respective binding partners. In certain aspects, the targeted protein for degradation may be a ubiquitin ligase.

Additional linkers L are disclosed in US Application Publication Nos. 2019/0175612, 2016/0058872; 2016/0045607; 2014/0356322; and 2015/0291562, and WO2014/063061.

In some embodiments, the compositions comprise a single PROTAC therapeutic agent. In some embodiments, the compositions comprise multiple PROTAC agents comprising any combination of the PROTAC agents described herein.

In some embodiments, PB group specifically binds one or more Bromodomain and Extra-Terminal motif (BET) proteins. BET is a subfamily of proteins responsible for recognition acetylated lysine residues, such as those on the N-terminal tails of histones, and include BRD2, BRD3, BRD4 and BRDT (see WO 2011/143669). In some embodiments, the BET-specific PB group comprises an anti-BET antibody. In some embodiments the BET-specific PB group comprises a BET inhibitor that is specific for one or more BET proteins. Numerous BET inhibitors are known in the art and are described, for example in Doroshow et al., Annals of Oncology, 2017, 28, 1776-1787; Pérez-Salviaa and Esteller Epigenetics, 2017, 12, 323-339; Klein, RMD Open, 2018, 4:e000744; Ocana et al., Oncotarget, 2017, 8, 71285-71291; Hogg et al., Blood, 2017, 130, 2537; U.S. Patent Application Publications 2018/0117030, 2016/0095867, 2018/0117165, PCT Publications WO 2011/054843, WO 2009/084693, 20180117165, and JP2008-156311.

In some embodiments, the BET inhibitors include, without limitation, olinone, JQ1, iBET, RVX-208, PF-1, ABBV-075, BAY1238097, BI 894999, BMS-986158, CPI-0610, FT-1101, GS-5829, GSK525762/I-BET762, GSK2820151/1-BET151, INCB054329, OTX015/MK-8628, PLX51107, R06870810/TEN-010, ZEN003694, CP1203, PFI-1, MS436, RVX2135, BAY-299, I-BET762, RVX297, SF1126, INCB054329, INCB057643, R06870810, LY294002, AZD5153, MT-1, MS645 and RG6146.

Multiple BET-specific POTACS assembled from, inter alia, the components described above are described in, for example in Sun et al., Leukemia, 2018, 32, 343-352; Zhang et al., Mol. Cancer Ther., 2019, 18, 1302-1311; Yang et al., Drug Discovery Today: Technologies, 2019, 31, 43-51; Raina et al., PNAS, 2016, 113, 7124-7129; and Zou et al, Cell Biochem Funct., 2019, 37, 21-30; An and Fu, EBioMedicine, 2018, 36, 553-562.

In some embodiments, the BET-PROTACS include, without limitation, MZ1, ARV-771, AT1, MZP-61, MZP-51, MZP-55, ARV825, dBET6, dBET57, dBET23, ZXH 3-26, BETd246, BETd260, QCA570, or ARCC-29, A1874, and CFT-2718.

In some embodiments PB group specifically binds Cyclin-Dependent Kinase 9 (CDK9) Protein. In some embodiments, the CDK9-specific PB group comprises an anti-CDK9 antibody. In some embodiments the CDK9-specific PB group comprises a CDK9 inhibitor. Numerous BET inhibitors are described, for example in Krystof, Medicinal Research Reviews, 2009, DOI 10.1002/med.24172; U.S. Patent Application Publications 2017/0304315, 2017/0173021, 2015/0329537, 2014/0287454, and PCT Publcations WO 2009/047359, WO 2010/003133, WO 2008/79933 and WO 2011/012661.

In some embodiments, the CDK9 inhibitors include, without limitation, NVP-2, LDCO000067, SNS-032 (BMS-387032), AT7519, P-276-00, AZD5438, PHA-767491, PHA-793887, PHA-848125, BAY 1143572, BAY 1112054, Cdk9 inhibitor II (CAS 140651-18-9 from Calbiochem), DRB, AZD-5438, SNS-032, dinaciclib, LY2857785, flavopiridol, purvalanol B, CDKI-71, CDKI-73, CAN508, FIT-039, CYC065, Ro-3306, 3,4-dimethyl-5-[2-(4-piperazin-1-yl-phenylamino)-pyrimidin-4-yl]-3H-thiazol-2-one, wogonin, apigenin, chrysin, luteolin, 4-methyl-5-[2-(3-nitroanilino)pyrimidin-4-yl]-1,3-thiazol-2-amine, 1073485-20-7P and Cpd B1.

Multiple CDK9-specific POTACS assembled, inter alia, from the components described above are described in, for example in Olson et al., Nat. Chem. Biol., 2018, 14, 163-170 and Robb et al, Chem. Commun. (Camb), 2017, 53, 7577-7580.

In some embodiments, the CDK9-PROTACS include, without limitation, THAL SNS 032, PROTAC3, and CDK9 Degrader-1.

In some embodiments, the PROTAC is a VHL-based PROTAC. In some embodiments, the PROTAC is a CRBN-based PROTAC.

In some embodiments, the PROTAC is any one or more selected from: MZ 1, ARV-825, dBET6, ARV-771, ARV-471, BSJ-4-116, XY028-140, BSJ-03-123, Gefitinib-based PROTAC 3, DT2216, LC-2, MD-224, BETd-260, SD-36, THAL-SNS-032, BRD4 degrader AT1, A1874, ZXH-3-26, dTRIM24, dBET57, MT-802, ACBI1, GNE-987, ARCC-4, CP-10, ARD-266, SJF620, XZ739, UNC6852, dFKBP-1, PROTAC FAK degrader 1, PROTAC Mcl1 degrader-1, PROTAC B-Raf degrader 1, PROTAC SGK3 degrader-1, FKBP12 PROTAC dTAG-13, BI-3663, PROTAC RIPK degrader-2, SIAIS178, BSJ-03-204, MZP-55, MS4078, PROTAC Sirt2 Degrader-1, JH-XI-10-02, GMB-475, PROTAC ERα Degrader-2, dCBP-1, BSJ-04-132, SJF620 hydrochloride, VZ185, CPSV, ERD-308, MD-222, ARD-2128, MS432, INY-03-041, MS938, JB170, CG 858, DD 03-171, dTRIM 24, FC 11, MS 154, GMB 475, ND1-YL2, SJF 0628, SJF 1521, SJF 1528, SJF 8240, ZNL 02-096, TL 13-12, TBK1 PROTAC® 3i, ARV-110, ARV-766, and ARV-V7.

In some embodiments, the PROTAC is any one or more E3 ligases selected from: CRBN, VHL, XIAP, cIAP, MDM2, KEAP1, DCAF15, RNF4, RNF114, DCAF16, UBR2, SPOP, KLHL3, KLH12, KLHL20, SPSB1, SPSB2, SPSB4, SOCS2, SOCS6, FBXO4, FBXO31, BTRC, FBW7, CDC20, ITCH, PML, TRIM21, TRIM24, TRIM33, and GID4.

In some embodiments, the PROTAC is not a BRD4-targeting PROTAC (such as dBET6). In some embodiments, the PROTAC is not Thal-SNS-032. In some embodiments, the PROTAC is not a VHL-mediated degrader of BRD4/3/2, ARV-771, and MZ-1.

In some embodiments, the compositions further comprise one or more kinase inhibitors, one or more KRAS inhibitors, or one or more autophagy inhibitors, or any combination thereof.

In some embodiments the kinase pathway inhibitors inhibit kinase signaling pathways including, without limitation, a receptor tyrosine kinase (RTK) pathway inhibitor, a mammalian target of rapamycin (MTOR) pathway inhibitor, or a CDK7 pathway inhibitor. RTK family of kinases are described, for example, by Bertrand et al., The International Journal of Developmental Biology, 61(10-11-12), 697-722 and Regad, Cancers 2015, 7, 1758-1784. In addition to RTK, the family includes, inter alia, Insulin Receptor (INSR/IGF1R), Epidermal Growth Factor Receptor family (EGFR/ERBB/HER2/HER3/HER4), Proto-Oncogene Tyrosine-Protein Kinase (MERTK), Macrophage-Stimulating Protein Receptor 1 (MST1R) and Fibroblast Growth Factor Receptor 1 (FGFR1), any of which can be targeted for inhibition according to the methods of the present disclosure. In some embodiments, the RTK pathway inhibitor targets RTK directly. RTK inhibitors include, without limitation, GTP14564, R 1530, imatinib, Sorafenib, Pazopanib, Cabozantinib, Sunitinib, Crizitonib, Regorafenib, and Dovitinib. In some embodiments, the RTK pathway inhibitor is an EGFR inhibitor. EGFR inhibitors include, without limitation, Trastuzumab, Panitumumab, Cetuximab, Afatinib, Sapitinib, Neratinib, Theliatinib, Avitinib, Canertinib, AG-490, CP-724714, Dacomitinib, WZ4002, CUDC-101, AG-1478, PD153035, Pelitinib, AC480, AEE788, OSI-420, WZ3146, ARRY-380, AST-1306, Rociletinib, Genistein, Varlitinib, Icotinib, TAK-285, WHI-P154, Daphnetin, PD168393, Tyrphostin 9, CNX-2006, AG-18, AZ5104, Osimertinib, CL-387785, Olmutinib, (−)-Epigallocatechin Gallate(EGCG), AZD3759, Poziotinib, Naquotinib, Chrysophanol, Nazartinib, Norcantharidin, Lifirafenib, Lidocaine hydrochloride, Butein, EAI045, NSC228155, Erlotinib, Gefitinib, and Lapatinib. In some embodiments, the RTK pathway inhibitor is an MERTK inhibitor. MERTK inhibitors include, without limitation, UNC1062, MRX-2843, RXDX-106, UNC2541, and unc2025. In some embodiments, the RTK pathway inhibitor is an MST1R inhibitor. MST1R inhibitors include, without limitation, LY2801653 dihydrochloride, BMS777607, and PHA 665752. In some embodiments, the RTK pathway inhibitor is an FGFR1 inhibitor. FGFRlinhibitors include, without limitation, Ponatinib, BGJ398, Nintedanib, PD173074, Dovitinib, Alofanib, Gambogenic Acid, Derazantinib, Nintedanib, AZD4547, Danusertib, Brivanib, Dovitinib Dilactic acid, Dovitinib Lactate, MK-2461, SSR128129E, LY2874455, H3B-6527, Erdafitinib, NSC12, 549076, BLU-554, PRN1371, PD-166866, PD-166866, FIIN-2, CH5183284, BLU9931, and SUN11602.

mTOR pathway inhibitors are described, for example, by Harter et al., PLoS ONE, 2015, 10, e0127123. In addition to MTOR, the signaling pathway includes, inter alia, Phosphoinositide 3-Kinase (PI3K), p70S6 kinase (P70S6K), Phosphatidylinositol 4-Kinase Type 2 Alpha (PI4K2A), and CDK9, any of which can be targeted for inhibition according to the methods of the present disclosure. In some embodiments, the mTOR inhibitor targets the mTOR kinase directly. mTOR inhibitors include, without limitation, Dactolisib (BEZ235 or NVP-BEZ235), Rapamycin (Sirolimus), Everolimus (RAD001), AZD8055, Temsirolimus (CCI-779 or NSC 683864), PI-103, KU-0063794, Torkinib (PP242), Tacrolimus (FK506), Ridaforolimus (Deforolimus or MK-8669), Sapanisertib (INK 128, MLN0128, or TAK-228), Voxtalisib (SAR245409 or XL765) Analogue, Torin 1, Omipalisib (GSK2126458 or GSK458), OSI-027, PF-04691502, Apitolisib (GDC-0980 or RG7422), GSK1059615, WYE-354, Gedatolisib (PF-05212384 or PKI-587), Torin 2, WYE-125132 (WYE-132), Vistusertib (AZD2014), BGT226 (NVP-BGT226), Palomid 529 (P529), PP121,WYE-687, WAY-600, ETP-46464, GDC-0349, XL388, Zotarolimus (ABT-578), LY3023414, CC-115, MHY1485, CZ415, GDC-0084, Voxtalisib (XL765 or SAR245409), 3BDO, Bimiralisib (PQR309), CC-223, and SF2523. In some embodiments, the mTOR pathway inhibitor is an PI3K inhibitor. PI3K inhibitors include, without limitation, compound 7n, Dactolisib, Pictilisib (GDC-0941), LY294002, Buparlisib, IC-87114, Wortmannin, XL147 analogue, ZSTK474, Apitolisib, AS-605240, 3-Methyladenine, PIK-90, PF-04691502, AZD6482, Apitolisib, GSK1059615, Duvelisib, Gedatolisib, TG100-115, AS-252424, BGT226, CUDC-907, AS-604850, GSK2636771, Copanlisib, CH5132799, CAY10505, PIK-293, PKI-402, TG100713, VS-5584, Taselisib, CZC24832, SF2523, AZD8835, AMG319, Seletalisib, TGR-1202, Pilaralisib, Bimiralisib, IPI-549, GDC-0084, Voxtalisib, HS-173, PF-4989216, Tenalisib, 740 Y-P, Leniolisib, GNE-317, LY3023414, GSK2292767, AZD8186, 2-D08, Nemiralisib, PI-103, Vistusertib, BGT226, and CC-223. In some embodiments, the mTOR pathway inhibitor is an P70S6K inhibitor. P70S6K inhibitors include, without limitation, BI-D1870, AT7867, PF-4708671, AD80, AT13148, LY2584702, and LY2584702 Tosylate. In some embodiments, the mTOR pathway inhibitor is an PI4K2A inhibitor. PI4K2A inhibitors include, without limitation, PIK-93, PI-273, NA04, NB04, NE02, NC02, NC03, NB02, NC04, ND02, NE03, NF03, NF04, NG02, NG03, NH02, NC02-567, and NC02-770 (see Sengupta et al., 2019 The Journal of Lipid Research, 60(3):683-693). In some embodiments, the mTOR pathway inhibitor is an CDK9 inhibitor. CDK9 inhibitors include, without limitation, NVP-2, LDC000067, SNS-032 (BMS-387032), AT7519, P-276-00, AZD5438, PHA-767491, PHA-793887, PHA-848125, BAY 1143572, BAY 1112054, Cdk9 inhibitor II (CAS 140651-18-9 from Calbiochem), DRB, AZD-5438, SNS-032, dinaciclib, LY2857785, flavopiridol, purvalanol B, CDKI-71, CDKI-73, CAN508, FIT-039, CYC065, Ro-3306, 3,4-dimethyl-5-[2-(4-piperazin-1-yl-phenylamino)-pyrimidin-4-yl]-3H-thiazol-2-one, wogonin, apigenin, chrysin, luteolin, 4-methyl-5-[2-(3-nitroanilino)pyrimidin-4-yl]-1,3-thiazol-2-amine, 1073485-20-7P and Cpd Bl.

The combination therapies described herein are considered to be global to improve the efficacy of PROTACs independent of the therapeutic target. Thus, MTOR inhibitors can be combined with any and all PROTACs, not just BET protein or CDK9 PROTACs.

In some embodiments, the kinase inhibitor is a CDK7 inhibitor. CDK7 inhibitors include, without limitation, THZ1, AT7519, LDC4297, LY2857785, BS-181 HC, SNS-032, R547, Flavopiridol, AT7519, PHA-793887, and Flavopiridol-HCl.

In some embodiments, the kinase inhibitor is a Casein kinase 2 (CK2) inhibitor. CK2 inhibitors include, without limitation, Silmitasertib, GSK269962, and TBB.

In some embodiments, the kinase inhibitor is a RAC-alpha serine/threonine-protein kinase 1 and 2 (AKT1/2) inhibitor. AKT1/2 inhibitors include, without limitation, Akti-1/2, MK-2206, Perifosine, GSK690693, Ipatasertib, AZD5363, AT7867, Triciribine, CCT128930, A-674563, Miltefosine, TIC10, SC66, Afuresertib, AT13148, Uprosertib, SC79, and ARQ 092.

In some embodiments, the kinase inhibitor is an Adaptor-associated kinase 1 (AAK1) inhibitor. AAK1 inhibitors include, without limitation, LP-935509, LP-922761, BMT-090605, BMT-124110, LP-927443, and BMS-901715 (see, Kostich et al., J. Pharmacol. Exp. Ther., 2016, 358, 371-386).

In some embodiments, the kinase inhibitor is a focal adhesion kinase (FAK1) inhibitor. FAKI1 inhibitors include, without limitation, PF-00562271, PF-573228, TAE226, PF-03814735, Defactinib, GSK2256098, PF-431396, Y15, and PND-1186.

In some embodiments, the kinase inhibitor is a mitogen-activated protein kinase kinase (MEK) inhibitor. MEK inhibitors include, without limitation, Binimetinib, Selumetinib, PD0325901, Trametinib, U0126-EtOH, PD184352, PD98059, Pimasertib, TAK-733, AZD8330, PD318088, SL327, Refametinib, GDC-0623, Cobimetinib, and BI-847325.

In some embodiments, the kinase inhibitor is a Rapidly Accelerated Fibrosarcoma (RAF) kinase inhibitor. RAF kinase inhibitors include, without limitation, Sorafenib, Vemurafenib, Regorafenib, Sorafenib Tosylate, Dabrafenib, AZ304, Belvarafenib, ERK-IN-1, PLX8394, Doramapimod, PLX-4720, LY3009120, RAF265, GW 5074, Dabrafenib Mesylate, LXH254, Agerafenib, GDC-0879, AZ 628, Ro 5126766, TAK-632, Regorafenib Hydrochloride, PLX7904, CCT196969, HG6-64-1, TAK-580, ZM 336372, AD80, SB-590885, Regorafenib monohydrate, Lifirafenib, L-779450, P-0850, B-Raf inhibitor 1, Belvarafenib, B-Raf IN 1, LUT014, BI-882370, Agerafenib hydrochloride, and B-Raf inhibitor 1 dihydrochloride.

In some embodiments, the compositions further comprise a KRAS inhibitor. KRAS inhibitors include, without limitation, K-Ras(G12C) inhibitors 1-12, Olaparib, AMG-510, Deltarasin, 6H05, ARS-1620, KRpep-2d, ARS-853, Lonafamib, MRTX-1257, PHT-7.3, ARS-1323 and MRTX849.

In some embodiments, the compositions further comprise an autophagy inhibitor. Autophagy inhibitors include, without limitation, Nimodipine, Lucanthone, Liensinine, Autophinib, DC661, EAD1, Spautin-1, ROC-325, PIK-III, PHY34, MHY1485, Hydroxychloroquine Sulfate, CA-5f, Bafilomycin Al, Daurisoline, 3BDO, SAR405, Elaiophylin, Autogramin-2, Lys05, DC661, IITZ-01, SBI-0206965, AS1842856, Chloroquine, 3-Methyladenine, ULK-101, J22352, LYN-1604, MRT68921 HC1, and hydroxychloroquine.

The ABC transport inhibitors can be any agent that inhibits any one or more of the following ABC transporter genes: ABCA1, ABCA10, ABCA12, ABCA2, ABCA3, ABCA4, ABCA5, ABCA6, ABCA7, ABCA8, ABCA9, ABCB1 (MDR1) (P-gp), ABCB10, ABCB11, ABCB2, ABCB3, ABCB4, ABCB5, ABCB6, ABCB7, ABCB8, ABCB9, ABCC1, ABCC10, ABCC11, ABCC12, ABCC2, ABCC3, ABCC4, ABCC5, ABCC6, ABCC7, ABCC8, ABCC9, ABCD1, ABCD2, ABCD3, ABCE1, ABCF1, ABCF2, ABCF3, ABCG1, ABCG2, ABCG4, ABCG5, and ABCG8.

In some embodiments, the ABC transport inhibitor is any one or more ABCB1 (MDR1) inhibitors selected from: Abemaciclib, Acetaminophen, Afatinib, Alectinib, Alfentanil, Alpelisib, Amiodarone, Amlodipine, Amodiaquine, Amoxapine, Amsacrine, Annamycin, Arsenic trioxide, Astemizole, Asunaprevir, Atazanavir, Atorvastatin, Atovaquone, Avapritinib, Axitinib, Azelastine, Azilsartan medoxomil, Azithromycin, Belumosudil, Benzocaine, Benzquinamide, Bepridil, Berotralstat, Bicalutamide, Biricodar, Bisoprolol, Boceprevir, Bosutinib, Brefeldin A, Bromocriptine, Buprenorphine, Buspirone, Cabazitaxel, Canagliflozin, Candesartan, Candesartan cilexetil, Capmatinib, Captopril, Carfilzomib, Carvedilol, Caspofungin, Ceftriaxone, Cethromycin, Cetirizine, Chloroquine, Chlorpromazine, Chlorprothixene, Cholesterol, Cilazapril, Citalopram, Clarithromycin, Clofazimine, Clomipramine, Clotrimazole, Cobicistat, Colforsin, Concanamycin A, Conivaptan, Crizotinib, Curcumin, Cyclosporine, Daclatasvir, Dacomitinib, Dactinomycin, Darunavir, Dasatinib, Daunorubicin, Desipramine, Desloratadine, Desmethylsertraline, Dexamethasone, Dexamethasone acetate, Dexniguldipine, Dexverapamil, Digoxin, Dihydroergotamine, Diltiazem, Diosmin, Dipyridamole, Dofequidar, Dovitinib, Doxazosin, Doxorubicin, Dronabinol, Dronedarone, Duloxetine, Econazole, Elacridar, Elagolix, Elbasvir, Elexacaftor, Eliglustat, Emopamil, Enalapril, Enasidenib, Entrectinib, Enzalutamide, Erdafitinib, Ergometrine, Ergotamine, Erlotinib, Erythromycin, Esomeprazole, Estramustine, Etoposide, Etravirine, Everolimus, Favipiravir, Fedratinib, Felodipine, Fenofibrate, Fentanyl, Fingolimod, Flibanserin, Fluconazole, Fluoxetine, Flupentixol, Fluphenazine, Flurazepam, Fluvoxamine, Galantamine, Gallopamil, Gefitinib, Genistein, Glasdegib, Glecaprevir, Glyburide, Gramicidin D, Grepafloxacin, Haloperidol, HM-30181, Hycanthone, Hydroxychloroquine, Ibuprofen, Idelalisib, Imatinib, Indinavir, Indomethacin, Infigratinib, Isavuconazole, Isavuconazonium, Isradipine, Istradefylline, Itraconazole, Ivacaftor, Ivermectin, Ivosidenib, Ixabepilone, Ketoconazole, Lamotrigine, Laniquidar, Lansoprazole, Lapatinib, Lasmiditan, Ledipasvir, Lenvatinib, Letermovir, Levofloxacin, Levoketoconazole, Lidocaine, Linagliptin, Lomerizine, Lomitapide, Lonafamib, Lopinavir, Loratadine, Losartan, Lovastatin, Loxapine, Lumacaftor, Lurbinectedin, Medroxyprogesterone acetate, Mefloquine, Megestrol acetate, Methadone, Methylene blue, Metronidazole, Mibefradil, Miconazole, Mifepristone, Mirabegron, Mitotane, Mitoxantrone, Mobocertinib, Monensin, Naproxen, Nefazodone, Nelfinavir, Neratinib, Netupitant, Nicardipine, Nifedipine, Niguldipine, Nilotinib, Nimodipine, Nintedanib, Nisoldipine, Nitrendipine, Norethisterone, Norgestimate, Olaparib, Omeprazole, ONT-093, Paclitaxel, Palbociclib, Paliperidone, Pantoprazole, Paritaprevir, Paroxetine, Pemigatinib, Pibrentasvir, Pimozide, Piperine, Polyethylene glycol, Polyethylene glycol 400, Ponatinib, Posaconazole, Pralsetinib, Prazosin, Prednisone, Primaquine, Progesterone, Promethazine, Propafenone, Propranolol, Protriptyline, Quercetin, Quinacrine, Quinidine, Quinine, Ranitidine, Ranolazine, Reboxetine, Regorafenib, Relugolix, Reserpine, Reversin 121, Rifamycin, Rilpivirine, Ripretinib, Ritonavir, Rolapitant, Rucaparib, Salinomycin, Sapropterin, Saquinavir, Sarecycline, Selegiline, Selpercatinib, Sertraline, Sildenafil, Simeprevir, Simvastatin, Sirolimus, Sorafenib, Sotorasib, Staurosporine, Sunitinib, Suvorexant, Tacrolimus, Tamoxifen, Tariquidar, Taurocholic acid, Telaprevir, Telmisartan, Temsirolimus, Tenofovir disoproxil, Tepotinib, Terazosin, Terfenadine, Tesmilifene, Testosterone, Testosterone cypionate, Testosterone enanthate, Testosterone undecanoate, Tetrandrine, Tezacaftor, Ticagrelor, Tipifarnib, Tipranavir, Tolvaptan, Toremifene, Trifluoperazine, Triflupromazine, Trimethoprim, Troleandomycin, Tucatinib, Umbralisib, Upadacitinib, Valinomycin, Valspodar, Vandetanib, Vardenafil, Velpatasvir, Vemurafenib, Venetoclax, Venlafaxine, Verapamil, Vinblastine, Vincristine, Voacamine, Voclosporin, Vorapaxar, Voxilaprevir, Yohimbine, Zimelidine, Zonisamide, Zosuquidar, Elagolix-stradiol orethindrone, Elexacaftor-tezacaftor-ivacaftor, Fostamatinib, Ombitasvir-paritaprevir-itonavir (Technivie)*, Osimertinib, Ritonavir and ritonavir-containing coformulations*, Tamoxifen, Tezacaftor-ivacaftor, Glecaprevir-pibrentasvir, Elacridar, Valspdar, Encequidar, XR9051, and YS-370.

The dual ABC transport/kinase inhibitors can be any one or more of the following: Afatinib, Dacomitinib, Dovitinib, Erdafitinib, Erlotinib, Everolimus, Gefitinib, Imatinib, Lapatinib, Neratinib, Nintedanib, Ponatinib, Regorafenib, Sirolimus, Sorafenib, Sunitinib, Temsirolimus, Vemurafenib, Osimertinib, Pelitinib, WZ3146, WZ4002, Deforolimus, Rapamycin, WYE-687, WAY-600, BEZ235, Cabozantinib, Canertinib, Pazopanib, and Icotinib. In some embodiments, the dual ABC transport/kinase inhibitor is Lapatinib.

The dual ABC transport/kinase inhibitors can be any one or more of the following: Abemaciclib, Alectinib, Avapritinib, Axitinib, Bosutinib, Crizotinib, Entrectinib, Fedratinib, Idelalisib, Lenvatinib, Mobocertinib, Nilotinib, Palbociclib, Ripretinib, Selpercatinib, Sotorasib, Tepotinib, Tucatinib, Umbralisib, Upadacitinib, Vandetanib, Fostamatinib, Voruciclib, Purvalanol A, Olomoucine II, Roscovitine, NVP-TAE684, SNS-314, LY2603618, MP-470, Masitinib, Ki8751, BIBW2992, TG101209, CI-1033, NVP-ADW742, NVP-BSK805, Crenolanib, B12536, GSK461364, B16727, AZD0530, Cediranib, AV-951, Ceritinib, Motesanib, Saracatinib, Vatalanib, Apatinib, Dasatinib, Ibrutinib, Linsitinib, Quizartinib, Tandutinib, Vatalinib, and Telatinib.

In any of the embodiments described herein, the PROTAC therapeutic agent can be linked to the ABC transporter inhibitor, the kinase inhibitor, or the dual ABC transporter/kinase inhibitor to form a single agent (i.e., a “caged molecule”). In some embodiments, the PROTAC therapeutic agent can be linked to the ABC transporter inhibitor. In some embodiments, the PROTAC therapeutic agent can be linked to the kinase inhibitor. In some embodiments, the PROTAC therapeutic agent can be linked to the dual ABC transporter/kinase inhibitor. These caged compounds can be prepared by designing a kinase inhibitor-, ABC transporter inhibitor-, or dual ABC/kinase inhibitor-caged pomalidomide prodrug that combines the inhibitor with a PROTAC targeting a protein of interest using a reduction-cleavable disulfide liker (see, Chen et al., J. Med. Chem., 2021, 64, 12273-12285). When the prodrug enters a cell, the linker is cleaved freeing the PROTAC and ABC/kinase inhibitor (or kinase inhibitor or ABC transporter inhibitor) to engage the PROTAC target and block ABCB1-driven resistance.

In some embodiments, the compositions comprise one or more BET-PROTAC therapeutic agents and one or more MTOR signaling pathway inhibitors. In some embodiments, the compositions comprise MZ1 and one or more MTOR signaling pathway inhibitors. In some embodiments, the compositions comprise MZ1 and one or more of RAD001, Torin-1, GDC-0941, LY2584702, PI-273, or NVP-2. In some embodiments, the compositions comprise MZ1 and GDC-0941. In some embodiments, the compositions comprise MZ1 and lapatinib. In some embodiments, the compositions comprise MZ1 and RAD001. In some embodiments, the compositions comprise MZ1 and LY2584702. In some embodiments, the compositions comprise MZ1 and PI-273. In some embodiments, the compositions comprise MZ1 and NVP-2. In some embodiments, the compositions comprise ARV825 and one or more MTOR signaling pathway inhibitors. In some embodiments, the compositions comprise ARV825 and one or more of RAD001, Torin-1, GDC-0941, LY2584702, PI-273, or NVP-2. In some embodiments, the compositions comprise ARV825 and GDC-0941. In some embodiments, the compositions comprise ARV825 and lapatinib. In some embodiments, the compositions comprise ARV825 and RAD001. In some embodiments, the compositions comprise ARV825 and LY2584702. In some embodiments, the compositions comprise ARV825 and PI-273. In some embodiments, the compositions comprise ARV825 and NVP-2. In some embodiments, the compositions comprise dBET1 and one or more MTOR signaling pathway inhibitors. In some embodiments, the compositions comprise dBET1 and one or more of RAD001, Torin-1, GDC-0941, LY2584702, PI-273, or NVP-2. In some embodiments, the compositions comprise dBET1 and GDC-0941. In some embodiments, the compositions comprise dBET1 and lapatinib. In some embodiments, the compositions comprise dBET1 and RAD001. In some embodiments, the compositions comprise dBET1 and LY2584702. In some embodiments, the compositions comprise dBET1 and PI-273. In some embodiments, the compositions comprise dBET1 and NVP-2. In some embodiments, the compositions comprise A1874 and one or more MTOR signaling pathway inhibitors. In some embodiments, the compositions comprise A1874 and one or more of RAD001, Torin-1, GDC-0941, LY2584702, PI-273, or NVP-2. In some embodiments, the compositions comprise A1874 and GDC-0941. In some embodiments, the compositions comprise A1874 and lapatinib. In some embodiments, the compositions comprise A1874 and RAD001. In some embodiments, the compositions comprise A1874 and LY2584702. In some embodiments, the compositions comprise A1874 and PI-273. In some embodiments, the compositions comprise A1874 and NVP-2. In some embodiments, the compositions comprise CFT-2718 and one or more MTOR signaling pathway inhibitors. In some embodiments, the compositions comprise CFT-2718 and one or more of RAD001, Torin-1, GDC-0941, LY2584702, PI-273, or NVP-2. In some embodiments, the compositions comprise CFT-2718 and GDC-0941. In some embodiments, the compositions comprise CFT-2718 and lapatinib. In some embodiments, the compositions comprise CFT-2718 and RAD001. In some embodiments, the compositions comprise CFT-2718 and LY2584702. In some embodiments, the compositions comprise CFT-2718 and PI-273. In some embodiments, the compositions comprise CFT-2718 and NVP-2.

In some embodiments, the compositions comprise one or more BET-PROTAC therapeutic agents and one or more RTK signaling pathway inhibitors. In some embodiments, the compositions comprise MZ1 and one or more RTK signaling pathway inhibitors. In some embodiments, the compositions comprise MZ1 and one or more of GSK1904529A, lapatinib, imatinib, MRX-2843, LY2801653 dihydrochloride and PD173074. In some embodiments, the compositions comprise MZ1 and GSK1904529A. In some embodiments, the compositions comprise MZ1 and lapatinib. In some embodiments, the compositions comprise MZ1 and imatinib. In some embodiments, the compositions comprise MZ1 and MRX-2843. In some embodiments, the compositions comprise MZ1 and LY2801653 dihydrochloride. In some embodiments, the compositions comprise MZ1 and PD173074. In some embodiments, the compositions comprise ARV825 and one or more RTK signaling pathway inhibitors. In some embodiments, the compositions comprise ARV825 and one or more of GSK1904529A, lapatinib, imatinib, MRX-2843, LY2801653 dihydrochloride and PD173074. In some embodiments, the compositions comprise ARV825 and GSK1904529A. In some embodiments, the compositions comprise ARV825 and lapatinib. In some embodiments, the compositions comprise ARV825 and imatinib. In some embodiments, the compositions comprise ARV825 and MRX-2843. In some embodiments, the compositions comprise ARV825 and LY2801653 dihydrochloride. In some embodiments, the compositions comprise ARV825 and PD173074. In some embodiments, the compositions comprise dBET1 and one or more RTK signaling pathway inhibitors. In some embodiments, the compositions comprise dBET1 and one or more of GSK1904529A, lapatinib, imatinib, MRX-2843, LY2801653 dihydrochloride and PD173074. In some embodiments, the compositions comprise dBET1 and GSK1904529A. In some embodiments, the compositions comprise dBET1 and lapatinib. In some embodiments, the compositions comprise dBET1 and imatinib. In some embodiments, the compositions comprise dBET1 and MRX-2843. In some embodiments, the compositions comprise dBET1 and LY2801653 dihydrochloride. In some embodiments, the compositions comprise dBET1 and PD173074. In some embodiments, the compositions comprise A1874 and one or more RTK signaling pathway inhibitors. In some embodiments, the compositions comprise A1874 and one or more of GSK1904529A, lapatinib, imatinib, MRX-2843, LY2801653 dihydrochloride and PD173074. In some embodiments, the compositions comprise A1874 and GSK1904529A. In some embodiments, the compositions comprise A1874 and lapatinib. In some embodiments, the compositions comprise A1874 and imatinib. In some embodiments, the compositions comprise A1874 and MRX-2843. In some embodiments, the compositions comprise A1874 and LY2801653 dihydrochloride. In some embodiments, the compositions comprise A1874 and PD173074. In some embodiments, the compositions comprise CFT-2718 and one or more RTK signaling pathway inhibitors. In some embodiments, the compositions comprise CFT-2718 and one or more of GSK1904529A, lapatinib, imatinib, MRX-2843, LY2801653 dihydrochloride and PD173074. In some embodiments, the compositions comprise CFT-2718 and GSK1904529A. In some embodiments, the compositions comprise CFT-2718 and lapatinib. In some embodiments, the compositions comprise CFT-2718 and imatinib. In some embodiments, the compositions comprise CFT-2718 and MRX-2843. In some embodiments, the compositions comprise CFT-2718 and LY2801653 dihydrochloride. In some embodiments, the compositions comprise CFT-2718 and PD173074.

In some embodiments, the compositions comprise one or more BET-PROTAC therapeutic agents and one or more KRAS inhibitors. In some embodiments, the compositions comprise MZ1 and one or more KRAS inhibitors. In some embodiments, the compositions comprise MZ1 and one or more of ARS-1620 and MRTX849. In some embodiments, the compositions comprise MZ1 and ARS-1620. In some embodiments, the compositions comprise MZ1 and MRTX849. In some embodiments, the compositions comprise ARV825 and one or more of ARS-1620 and MRTX849. In some embodiments, the compositions comprise ARV825 and ARS-1620. In some embodiments, the compositions comprise ARV825 and MRTX849. In some embodiments, the compositions comprise dBET1 and one or more KRAS inhibitors. In some embodiments, the compositions comprise dBET1 and one or more of ARS-1620 and MRTX849. In some embodiments, the compositions comprise dBET1 and ARS-1620. In some embodiments, the compositions comprise dBET1 and MRTX849. In some embodiments, the compositions comprise A1874 and one or more KRAS inhibitors. In some embodiments, the compositions comprise A1874 and one or more of ARS-1620 and MRTX849. In some embodiments, the compositions comprise A1874 and ARS-1620. In some embodiments, the compositions comprise A1874 and MRTX849. In some embodiments, the compositions comprise CFT-2718 and one or more KRAS inhibitors. In some embodiments, the compositions comprise CFT-2718 and one or more of ARS-1620 and MRTX849. In some embodiments, the compositions comprise CFT-2718 and ARS-1620. In some embodiments, the compositions comprise CFT-2718 and MRTX849.

In some embodiments, the compositions comprise one or more BET-PROTAC therapeutic agents and one or more autophagy inhibitors. In some embodiments, the compositions comprise MZ1 and one or more autophagy inhibitors. In some embodiments, the compositions comprise MZ1 and one or more of SAR405, Autophinib, PIK-III, LYN-1604, SBI-0206965, MRT68921 HCl, ULK-101, and hydroxychloroquine. In some embodiments, the compositions comprise MZ1 and SAR405. In some embodiments, the compositions comprise MZ1 and Autophinib. In some embodiments, the compositions comprise MZ1 and PIK-III. In some embodiments, the compositions comprise MZ1 and LYN-1604. In some embodiments, the compositions comprise MZ1 and SBI-0206965. In some embodiments, the compositions comprise MZ1 and MRT68921 HCl. In some embodiments, the compositions comprise MZ1 and ULK-101. In some embodiments, the compositions comprise MZ1 and hydroxychloroquine. In some embodiments, the compositions comprise ARV825 and one or more autophagy inhibitors. In some embodiments, the compositions comprise ARV825 and one or more of SAR405, Autophinib, PIK-III, LYN-1604, SBI-0206965, MRT68921 HCl, ULK-101, and hydroxychloroquine. In some embodiments, the compositions comprise ARV825 and SAR405. In some embodiments, the compositions comprise ARV825 and Autophinib. In some embodiments, the compositions comprise ARV825 and PIK-III. In some embodiments, the compositions comprise ARV825 and LYN-1604. In some embodiments, the compositions comprise ARV825 and SBI-0206965. In some embodiments, the compositions comprise ARV825 and MRT68921 HCl. In some embodiments, the compositions comprise ARV825 and ULK-101. In some embodiments, the compositions comprise ARV825 and hydroxychloroquine. In some embodiments, the compositions comprise dBET1 and one or more autophagy inhibitors. In some embodiments, the compositions comprise dBET1 and one or more of SAR405, Autophinib, PIK-III, LYN-1604, SBI-0206965, MRT68921 HCl, ULK-101, and hydroxychloroquine. In some embodiments, the compositions comprise dBET1 and SAR405. In some embodiments, the compositions comprise dBET1 and Autophinib. In some embodiments, the compositions comprise dBET1 and PIK-III. In some embodiments, the compositions comprise dBET1 and LYN-1604. In some embodiments, the compositions comprise dBET1 and SBI-0206965. In some embodiments, the compositions comprise dBET1 and MRT68921 HCl. In some embodiments, the compositions comprise dBET and ULK-101. In some embodiments, the compositions comprise dBET and hydroxychloroquine. In some embodiments, the compositions comprise A1874 and one or more autophagy inhibitors. In some embodiments, the compositions comprise A1874 and one or more of SAR405, Autophinib, PIK-III, LYN-1604, SBI-0206965, MRT68921 HCl, ULK-101, and hydroxychloroquine. In some embodiments, the compositions comprise A1874 and SAR405. In some embodiments, the compositions comprise A1874 and Autophinib. In some embodiments, the compositions comprise A1874 and PIK-III. In some embodiments, the compositions comprise A1874 and LYN-1604. In some embodiments, the compositions comprise A1874 and SBI-0206965. In some embodiments, the compositions comprise A1874 and MRT68921 HCl. In some embodiments, the compositions comprise A1874 and ULK-101. In some embodiments, the compositions comprise A1874 and hydroxychloroquine. In some embodiments, the compositions comprise CFT-2718 and one or more autophagy inhibitors. In some embodiments, the compositions comprise CFT-2718 and one or more of SAR405, Autophinib, PIK-III, LYN-1604, SBI-0206965, MRT68921 HCl, ULK-101, and hydroxychloroquine. In some embodiments, the compositions comprise CFT-2718 and SAR405. In some embodiments, the compositions comprise CFT-2718 and Autophinib. In some embodiments, the compositions comprise CFT-2718 and PIK-III. In some embodiments, the compositions comprise CFT-2718 and LYN-1604. In some embodiments, the compositions comprise CFT-2718 and SBI-0206965. In some embodiments, the compositions comprise CFT-2718 and MRT68921 HCl. In some embodiments, the compositions comprise CFT-2718 and ULK-101. In some embodiments, the compositions comprise CFT-2718 and hydroxychloroquine.

In some embodiments, the compositions comprise one or more CDK9-PROTAC therapeutic agents and one or more MTOR signaling pathway inhibitors. In some embodiments, the compositions comprise THAL SNS 032 and one or more MTOR signaling pathway inhibitors. In some embodiments, the compositions comprise THAL SNS 032 and one or more of RAD001, Torin-1, GDC-0941, LY2584702, PI-273, or NVP-2. In some embodiments, the compositions comprise THAL SNS 032 and GDC-0941. In some embodiments, the compositions comprise THAL SNS 032 and lapatinib. In some embodiments, the compositions comprise THAL SNS 032 and RAD001. In some embodiments, the compositions comprise THAL SNS 032 and LY2584702. In some embodiments, the compositions comprise THAL SNS 032 and PI-273. In some embodiments, the compositions comprise THAL SNS 032 and NVP-2. In some embodiments, the compositions comprise NVP-2 and one or more MTOR signaling pathway inhibitors. In some embodiments, the compositions comprise NVP-2 and one or more of RAD001, Torin-1, GDC-0941, LY2584702, or PI-273. In some embodiments, the compositions comprise NVP-2 and GDC-0941. In some embodiments, the compositions comprise NVP-2 and lapatinib. In some embodiments, the compositions comprise NVP-2 and RAD001. In some embodiments, the compositions comprise NVP-2 and LY2584702. In some embodiments, the compositions comprise NVP-2 and PI-273. In some embodiments, the compositions comprise CDK9 Degrader-1 and one or more MTOR signaling pathway inhibitors. In some embodiments, the compositions comprise CDK9 Degrader-1 and one or more of RAD001, Torin-1, GDC-0941, LY2584702, PT-273, or NVP-2. In some embodiments, the compositions comprise CDK9 Degrader-1 and GDC-0941. In some embodiments, the compositions comprise CDK9 Degrader-1 and lapatinib. In some embodiments, the compositions comprise CDK9 Degrader-1 and RAD001. In some embodiments, the compositions comprise CDK9 Degrader-1 and LY2584702. In some embodiments, the compositions comprise CDK9 Degrader-1 and PI-273. In some embodiments, the compositions comprise CDK9 Degrader-1 and NVP-2.

In some embodiments, the compositions comprise one or more CDK9-PROTAC therapeutic agents and one or more RTK signaling pathway inhibitors. In some embodiments, the compositions comprise THAL SNS 032 and one or more RTK signaling pathway inhibitors. In some embodiments, the compositions comprise THAL SNS 032 and one or more of GSK1904529A, lapatinib, imatinib, MRX-2843, LY2801653 dihydrochloride and PD173074. In some embodiments, the compositions comprise THAL SNS 032 and GSK1904529A. In some embodiments, the compositions comprise THAL SNS 032 and lapatinib. In some embodiments, the compositions comprise THAL SNS 032 and imatinib. In some embodiments, the compositions comprise THAL SNS 032 and MRX-2843. In some embodiments, the compositions comprise THAL SNS 032 and LY2801653 dihydrochloride. In some embodiments, the compositions comprise THAL SNS 032 and PD173074. In some embodiments, the compositions comprise NVP-2 and one or more RTK signaling pathway inhibitors. In some embodiments, the compositions comprise NVP-2 and one or more of GSK1904529A, lapatinib, imatinib, MRX-2843, LY2801653 dihydrochloride and PD173074. In some embodiments, the compositions comprise NVP-2 and GSK1904529A. In some embodiments, the compositions comprise NVP-2 and lapatinib. In some embodiments, the compositions comprise NVP-2 and imatinib. In some embodiments, the compositions comprise NVP-2 and MRX-2843. In some embodiments, the compositions comprise NVP-2 and LY2801653 dihydrochloride. In some embodiments, the compositions comprise NVP-2 and PD173074. In some embodiments, the compositions comprise CDK9 Degrader-1 and one or more RTK signaling pathway inhibitors. In some embodiments, the compositions comprise CDK9 Degrader-1 and one or more of GSK1904529A, lapatinib, imatinib, MRX-2843, LY2801653 dihydrochloride and PD173074. In some embodiments, the compositions comprise CDK9 Degrader-1 and GSK1904529A. In some embodiments, the compositions comprise CDK9 Degrader-1 and lapatinib. In some embodiments, the compositions comprise CDK9 Degrader-1 and imatinib. In some embodiments, the compositions comprise CDK9 Degrader-1 and MRX-2843. In some embodiments, the compositions comprise CDK9 Degrader-1 and LY2801653 dihydrochloride. In some embodiments, the compositions comprise CDK9 Degrader-1 and PD173074.

In some embodiments, the compositions comprise one or more CDK9-PROTAC therapeutic agents and one or more KRAS inhibitors. In some embodiments, the compositions comprise THAL SNS 032 and one or more KRAS inhibitors. In some embodiments, the compositions comprise THAL SNS 032 and one or more of ARS-1620 and MRTX849. In some embodiments, the compositions comprise THAL SNS 032 and ARS-1620. In some embodiments, the compositions comprise THAL SNS 032 and MRTX849. In some embodiments, the compositions comprise NVP-2 and one or more of ARS-1620 and MRTX849. In some embodiments, the compositions comprise NVP-2 and ARS-1620. In some embodiments, the compositions comprise NVP-2 and MRTX849. In some embodiments, the compositions comprise CDK9 Degrader-1 and one or more KRAS inhibitors. In some embodiments, the compositions comprise CDK9 Degrader-1 and one or more of ARS-1620 and MRTX849. In some embodiments, the compositions comprise CDK9 Degrader-1 and ARS-1620. In some embodiments, the compositions comprise CDK9 Degrader-1 and MRTX849.

In some embodiments, the compositions comprise one or more CDK9-PROTAC therapeutic agents and one or more autophagy inhibitors. In some embodiments, the compositions comprise THAL SNS 032 and one or more autophagy inhibitors. In some embodiments, the compositions comprise THAL SNS 032 and one or more of SAR405, Autophinib, PIK-III, LYN-1604, SBI-0206965, MRT68921 HCl, ULK-101, and hydroxychloroquine. In some embodiments, the compositions comprise THAL SNS 032 and SAR405. In some embodiments, the compositions comprise THAL SNS 032 and Autophinib. In some embodiments, the compositions comprise THAL SNS 032 and PIK-III. In some embodiments, the compositions comprise THAL SNS 032 and LYN-1604. In some embodiments, the compositions comprise THAL SNS 032 and SBI-0206965. In some embodiments, the compositions comprise THAL SNS 032 and MRT68921 HCl. In some embodiments, the compositions comprise THAL SNS 032 and ULK-101. In some embodiments, the compositions comprise THAL SNS 032 and hydroxychloroquine. In some embodiments, the compositions comprise NVP-2 and one or more autophagy inhibitors. In some embodiments, the compositions comprise NVP-2 and one or more of SAR405, Autophinib, PIK-III, LYN-1604, SBI-0206965, MRT68921 HCl, ULK-101, and hydroxychloroquine. In some embodiments, the compositions comprise NVP-2 and SAR405. In some embodiments, the compositions comprise NVP-2 and Autophinib. In some embodiments, the compositions comprise NVP-2 and PIK-III. In some embodiments, the compositions comprise NVP-2 and LYN-1604. In some embodiments, the compositions comprise NVP-2 and SBI-0206965. In some embodiments, the compositions comprise NVP-2 and MRT68921 HCl. In some embodiments, the compositions comprise NVP-2 and ULK-101. In some embodiments, the compositions comprise NVP-2 and hydroxychloroquine. In some embodiments, the compositions comprise CDK9 Degrader-1 and one or more autophagy inhibitors. In some embodiments, the compositions comprise CDK9 Degrader-1 and one or more of SAR405, Autophinib, PIK-III, LYN-1604, SBI-0206965, MRT68921 HCl, ULK-101, and hydroxychloroquine. In some embodiments, the compositions comprise CDK9 Degrader-1 and SAR405. In some embodiments, the compositions comprise CDK9 Degrader-1 and Autophinib. In some embodiments, the compositions comprise CDK9 Degrader-1 and PIK-III. In some embodiments, the compositions comprise CDK9 Degrader-1 and LYN-1604. In some embodiments, the compositions comprise CDK9 Degrader-1 and SBI-0206965. In some embodiments, the compositions comprise CDK9 Degrader-1 and MRT68921 HCl. In some embodiments, the compositions comprise CDK9 Degrader-1 and ULK-101. In some embodiments, the compositions comprise CDK9 Degrader-1 and hydroxychloroquine.

In some embodiments, the pharmaceutical composition comprises one or more PROTAC therapeutic agents, one or more kinase inhibitors, and one or more ABC transporter inhibitors. In some embodiments, the pharmaceutical composition comprises one or more PROTAC therapeutic agents and one or more dual ABC transporter/kinase inhibitors. In some embodiments, the PROTAC targets the EGFR-KRAS-PI3K-MEK signaling pathway. In some embodiments, the PROTAC targets EGFR, HER2, HER3, PI3K, AKT, KRAS, RAF, MEK, or ERK. In some embodiments, the PROTAC targets EGFR. In some embodiments, the PROTAC targets HER2. In some embodiments, the PROTAC targets HER3. In some embodiments, the PROTAC targets PI3K. In some embodiments, the PROTAC targets AKT. In some embodiments, the PROTAC targets KRAS. In some embodiments, the PROTAC targets RAF. In some embodiments, the PROTAC targets MEK. In some embodiments, the PROTAC targets ERK. In some embodiments, the dual ABC transporter/kinase inhibitor is lapatinib or RAD001. In some embodiments, the dual ABC transporter/kinase inhibitor is lapatinib. In some embodiments, the dual ABC transporter/kinase inhibitor is RAD001.

The present disclosure further provides methods for augmenting the therapeutic effect in a human cancer patient undergoing treatment with a PROTAC therapeutic agent comprising administering one or more kinase inhibitors, one or more KRAS inhibitors, or one or more autophagy inhibitors, or any combination thereof, to the patient. In some embodiments, the PROTAC therapeutic agent is not a BET-PROTAC therapeutic agent or a CDK9-PROTAC therapeutic agent.

The present disclosure further provides methods for augmenting the therapeutic effect in a human cancer patient undergoing treatment with a PROTAC therapeutic agent comprising administering one or more kinase inhibitors, one or more ABC transporter inhibitors, or one or more dual ABC transporter/kinase inhibitors, or any combination thereof. The present disclosure further provides methods for augmenting the therapeutic effect in a human cancer patient undergoing treatment with a PROTAC therapeutic agent comprising administering one or more PROTAC therapeutic agents, and one or more dual ABC transporter/kinase inhibitors.

The present disclosure also provides methods of treating cancer in a human patient in need thereof, the method comprising administering to the patient one or more PROTAC therapeutic agents and one or more kinase inhibitors, one or more KRAS inhibitors, or one or more autophagy inhibitors, or any combination thereof. In some embodiments, the PROTAC therapeutic agent is not a BET-PROTAC therapeutic agent or a CDK9-PROTAC therapeutic agent.

The present disclosure also provides methods of treating cancer in a human patient in need thereof, the method comprising administering to the patient: i) one or more PROTAC therapeutic agents; and ii) one or more kinase inhibitors, one or more ABC transporter inhibitors, or one or more dual ABC transporter/kinase inhibitors, or any combination thereof. The present disclosure also provides methods of treating cancer in a human patient in need thereof, the method comprising administering to the patient one or more PROTAC therapeutic agents, and one or more dual ABC transporter/kinase inhibitors.

The present disclosure also provides methods of overcoming resistance to PROTAC therapeutic agents in a human patient, the method comprising administering to the patient: i) one or more PROTAC therapeutic agents; and ii) one or more kinase inhibitors, one or more KRAS inhibitors, or one or more autophagy inhibitors, or any combination thereof. In some embodiments, the PROTAC therapeutic agent is not a BET-PROTAC therapeutic agent or a CDK9-PROTAC therapeutic agent.

The present disclosure also provides methods of overcoming resistance to PROTAC therapeutic agents in a human patient, the method comprising administering to the patient: i) one or more PROTAC therapeutic agents; and ii) one or more kinase inhibitors, one or more ABC transporter inhibitors, or one or more dual ABC transporter/kinase inhibitors, or any combination thereof. The present disclosure also provides methods of overcoming resistance to PROTAC therapeutic agents in a human patient, the method comprising administering to the patient one or more PROTAC therapeutic agents, and one or more dual ABC transporter/kinase inhibitors.

In any of the methods described herein, a PROTAC therapeutic agent is linked to a kinase inhibitor in the form of a caged molecule. In any of the methods described herein, a PROTAC therapeutic agent is linked to an ABC transporter inhibitor in the form of a caged molecule. In any of the methods described herein, a PROTAC therapeutic agent is linked to a dual ABC transporter/kinase inhibitor in the form of a caged molecule.

In some embodiments, the PROTAC therapeutic agent is administered prior to the administration of the one or more kinase inhibitors, one or more KRAS inhibitors, one or more autophagy inhibitors, one or more ABC transporter inhibitors, or one or more dual ABC transporter/kinase inhibitors, or after administration of the one or more kinase inhibitors, one or more KRAS inhibitors, one or more autophagy inhibitors, one or more ABC transporter inhibitors, or one or more dual ABC transporter/kinase inhibitors. In some embodiments, the PROTAC therapeutic agent is administered prior to the administration of the one or more kinase inhibitors, one or more KRAS inhibitors, one or more autophagy inhibitors, one or more ABC transporter inhibitors, or one or more dual ABC transporter/kinase inhibitors. In some embodiments, the PROTAC therapeutic agent is administered after administration of the one or more kinase inhibitors, one or more KRAS inhibitors, one or more autophagy inhibitors, one or more ABC transporter inhibitors, or one or more dual ABC transporter/kinase inhibitors. In some embodiments, the PROTAC therapeutic agent is administered concurrently with administration of the one or more kinase inhibitors, one or more KRAS inhibitors, one or more autophagy inhibitors, one or more ABC transporter inhibitors, or one or more dual ABC transporter/kinase inhibitors, or any combination thereof.

The present disclosure also provides methods for killing or inhibiting growth of a cancer cell comprising comprising contacting the cancer cell with any of the combinations of compounds, or compositions comprising the same as described herein. In some embodiments, one or more compounds may be combined in the same composition for any of the methods disclosed herein.

Thus, the compounds and compositions can be used as anti-cancer and anti-tumor agents, e.g., the compounds can kill or inhibit the growth of cancer cells. The compounds and compositions can also be used in methods of reducing cancer in an animal, or in methods of treating or preventing the spread or metastasis of cancer in an animal, or in methods of treating an animal afflicted with cancer. The compounds and compositions can also be used in methods of killing or inhibiting the growth of a cancer cell, or in methods of inhibiting tumor growth.

Cancers that are treatable are broadly divided into the categories of carcinoma, lymphoma and sarcoma. Examples of carcinomas include, but are not limited to: adenocarcinoma, acinic cell adenocarcinoma, adrenal cortical carcinomas, alveoli cell carcinoma, anaplastic carcinoma, basaloid carcinoma, basal cell carcinoma, bronchiolar carcinoma, bronchogenic carcinoma, renaladinol carcinoma, embryonal carcinoma, anometroid carcinoma, fibrolamolar liver cell carcinoma, follicular carcinomas, giant cell carcinomas, hepatocellular carcinoma, intraepidermal carcinoma, intraepithelial carcinoma, leptomanigio carcinoma, medullary carcinoma, melanotic carcinoma, menigual carcinoma, mesometonephric carcinoma, oat cell carcinoma, squamal cell carcinoma, sweat gland carcinoma, transitional cell carcinoma, and tubular cell carcinoma. Sarcomas include, but are not limited to: amelioblastic sarcoma, angiolithic sarcoma, botryoid sarcoma, endometrial stroma sarcoma, ewing sarcoma, fascicular sarcoma, giant cell sarcoma, granulositic sarcoma, immunoblastic sarcoma, juxaccordial osteogenic sarcoma, coppices sarcoma, leukocytic sarcoma (leukemia), lymphatic sarcoma (lympho sarcoma), medullary sarcoma, myeloid sarcoma (granulocitic sarcoma), austiogenci sarcoma, periosteal sarcoma, reticulum cell sarcoma (histiocytic lymphoma), round cell sarcoma, spindle cell sarcoma, synovial sarcoma, and telangiectatic audiogenic sarcoma. Lymphomas include, but are not limited to: Hodgkin's disease and lymphocytic lymphomas, such as Burkitt's lymphoma, NPDL, NML, NH and diffuse lymphomas.

Examples of cancers which may be treated by the compositions include, but are not limited to acute myeloid leukemia, acute monocytic leukemia, prostatic adenocarcinoma, ovarian carcinoma, or epithelial ovarian cancer, such as High-Grade Serous Ovarian Carcinoma (HGSOC). In some embodiments, the cancers which may be treated by the compositions include KRAS cancers (including, but not limited to non-small cell lung cancer (NSCLC), colorectal cancer, and pancreatic cancer), PIK3CA cancers (including, but not limited to breast cancer, colon cancer, endometrial cancer, glioblastoma multiformes, epidermal nevi, seborrheic keratoses (SK), ovarian cancer, gastric cancer, squamous cell carcinoma, thyroid cancer, oral squamous cell carcinoma, nasopharyngeal carcinoma, cervical cancer, papillary mucinous carcinoma of the pancreas, squamous cell carcinoma of the esophagus, adenocarcinomas of the esophagus, gallbladder carcinoma, cholangiocarcinoma, invasive pituitary tumors, penile tumors, bladder cancer, and diffuse large B cell lymphomas) or PTEN cancers (including, but not limited to glioblastoma, endometrial cancer, breast cancer, Endometrial Cancer, and prostate cancer). Thus, cancer cells harboring K-Ras mutations or other mutations (such as Kras-amplification, PIK3CA-mutations, PTEN-loss, EGFR-mutants, HER2 overexpression, and AKT amplification) that strongly activate mTORC1 signaling can promote intrinsic resistance to BBDs representing a patient population that may benefit from combined mTORC1 and PROTAC-treatment.

The compounds and compositions can be used in methods of killing or inhibiting the growth of cancer cells, either in vivo or in vitro, or inhibiting the growth of a cancerous tumor.

In some embodiments, the compounds and compositions are used in conjunction with other therapies, such as standard immunotherapy, neoadjuvant therapy, radiotherapy, tumor surgery, and conventional chemotherapy directed against solid tumors and for the control of establishment of metastases. Additionally, the compounds and compositions can be administered after surgery where solid tumors have been removed as a prophylaxis against metastasis. Cytotoxic or chemotherapeutic agents include, but are ot limited to, aziridine thiotepa, alkyl sulfonate, nitrosoureas, platinum complexes, NO classic alkylators, folate analogs, purine analogs, adenosine analogs, pyrimidine analogs, substituted urea, antitumor antibiotics, microtubulle agents, and asparaginase.

In some embodiments, the subject is also administered radiation therapy, immunotherapy, and/or neoadjuvant therapy. In some embodiments, the subject is also administered radiation therapy. In some embodiments, the subject is also administered immunotherapy. In some embodiments, the subject is also administered neoadjuvant therapy.

The present disclosure provides pharmaceutical formulations comprising one or more PROTAC therapeutic agents; one or more kinase inhibitors, one or more KRAS inhibitors, or one or more autophagy inhibitors, or any combination thereof and a pharmaceutically acceptable carrier or excipient. In some embodiments, the PROTAC therapeutic agent is not a BET-PROTAC therapeutic agent or a CDK9-PROTAC therapeutic agent.

In some embodiments, the ratio of the PROTAC therapeutic agents to the one or more kinase inhibitors, one or more KRAS inhibitors, one or more autophagy inhibitors, one or more ABC transporter inhibitors, or one or more dual ABC transporter/kinase inhibitors is from about 0.01:1 to about 100:1 (w/w), from about 0.1:1 to about 10:1 (w/w), or from about 1:1 to about 5:1 (w/w). In some embodiments, the ratio of the PROTAC therapeutic agents to the one or more kinase inhibitors, one or more KRAS inhibitors, one or more autophagy inhibitors, one or more ABC transporter inhibitors, or one or more dual ABC transporter/kinase inhibitors is from about 0.01:1 to about 100:1 (w/w). In some embodiments, the ratio of the PROTAC therapeutic agents to the one or more kinase inhibitors, one or more KRAS inhibitors, one or more autophagy inhibitors, one or more ABC transporter inhibitors, or one or more dual ABC transporter/kinase inhibitors is from about 0.1:1 to about 10:1 (w/w). In some embodiments, the ratio of the PROTAC therapeutic agents to the one or more kinase inhibitors, one or more KRAS inhibitors, one or more autophagy inhibitors, one or more ABC transporter inhibitors, or one or more dual ABC transporter/kinase inhibitors is from about 1:1 to about 5:1 (w/w).

In some embodiments, the PROTAC therapeutic agent is present in an amount from about 1 mg to about 100 mg, from about 5 mg to about 75 mg, from about 10 mg to about 60 mg, or from about 12.5 mg to about 50 mg, and the one or more kinase inhibitors, one or more KRAS inhibitors, one or more autophagy inhibitors, one or more ABC transporter inhibitors, or one or more dual ABC transporter/kinase inhibitors is present in an amount from about 1 mg to about 500 mg, from about 50 mg to about 400 mg, from about 75 mg to about 300 mg, or from about 100 mg to about 200 mg. In some embodiments, the PROTAC therapeutic agent is present in an amount from about 1 mg to about 100 mg. In some embodiments, the PROTAC therapeutic agent is present in an amount from about 5 mg to about 75 mg. In some embodiments, the PROTAC therapeutic agent is present in an amount from about 10 mg to about 60 mg. In some embodiments, the PROTAC therapeutic agent is present in an amount from about 12.5 mg to about 50 mg. In some embodiments, the one or more kinase inhibitors, one or more KRAS inhibitors, or one or more autophagy inhibitors is present in an amount from about 1 mg to about 500 mg. In some embodiments, the one or more kinase inhibitors, one or more KRAS inhibitors, or one or more autophagy inhibitors is present in an amount from about 50 mg to about 400 mg. In some embodiments, the one or more kinase inhibitors, one or more KRAS inhibitors, or one or more autophagy inhibitors is present in an amount from about 75 mg to about 300 mg. In some embodiments, the one or more kinase inhibitors, one or more KRAS inhibitors, or one or more autophagy inhibitors is present in an amount from about 100 mg to about 200 mg.

In some embodiments, the amount of the PROTAC therapeutic agent administered to the human patient receiving administration of one or more kinase inhibitors, one or more KRAS inhibitors, one or more autophagy inhibitors, one or more ABC transporter inhibitors, or one or more dual ABC transporter/kinase inhibitors is reduced compared to the amount of the PROTAC therapeutic agent administered to the human patient in the absence of receiving administration of one or more kinase inhibitors, one or more KRAS inhibitors, one or more autophagy inhibitors, one or more ABC transporter inhibitors, or one or more dual ABC transporter/kinase inhibitors. In some embodiments, the amount of the PROTAC therapeutic agent administered to the human patient receiving administration of one or more kinase inhibitors, one or more KRAS inhibitors, one or more autophagy inhibitors, one or more ABC transporter inhibitors, or one or more dual ABC transporter/kinase inhibitors is reduced by 10%. In some embodiments, the amount of the PROTAC therapeutic agent administered to the human patient receiving administration of one or more kinase inhibitors, one or more KRAS inhibitors, one or more autophagy inhibitors, one or more ABC transporter inhibitors, or one or more dual ABC transporter/kinase inhibitors is reduced by 15%. In some embodiments, the amount of the PROTAC therapeutic agent administered to the human patient receiving administration of one or more kinase inhibitors, one or more KRAS inhibitors, one or more autophagy inhibitors, one or more ABC transporter inhibitors, or one or more dual ABC transporter/kinase inhibitors is reduced by 20%. In some embodiments, the amount of the PROTAC therapeutic agent administered to the human patient receiving administration of one or more kinase inhibitors, one or more KRAS inhibitors, one or more autophagy inhibitors, one or more ABC transporter inhibitors, or one or more dual ABC transporter/kinase inhibitors is reduced by 25%. In some embodiments, the amount of the PROTAC therapeutic agent administered to the human patient receiving administration of one or more kinase inhibitors, one or more KRAS inhibitors, one or more autophagy inhibitors, one or more ABC transporter inhibitors, or one or more dual ABC transporter/kinase inhibitors is reduced by 30%. In some embodiments, the amount of the PROTAC therapeutic agent administered to the human patient receiving administration of one or more kinase inhibitors, one or more KRAS inhibitors, one or more autophagy inhibitors, one or more ABC transporter inhibitors, or one or more dual ABC transporter/kinase inhibitors is reduced by 35%. In some embodiments, the amount of the PROTAC therapeutic agent administered to the human patient receiving administration of one or more kinase inhibitors, one or more KRAS inhibitors, one or more autophagy inhibitors, one or more ABC transporter inhibitors, or one or more dual ABC transporter/kinase inhibitors is reduced by 40%. In some embodiments, the amount of the PROTAC therapeutic agent administered to the human patient receiving administration of one or more kinase inhibitors, one or more KRAS inhibitors, one or more autophagy inhibitors, one or more ABC transporter inhibitors, or one or more dual ABC transporter/kinase inhibitors is reduced by 45%. In some embodiments, the amount of the PROTAC therapeutic agent administered to the human patient receiving administration of one or more kinase inhibitors, one or more KRAS inhibitors, one or more autophagy inhibitors, one or more ABC transporter inhibitors, or one or more dual ABC transporter/kinase inhibitors is reduced by 50% compared to the amount of the PROTAC therapeutic agent administered to the human patient in the absence of receiving administration of one or more kinase inhibitors, one or more KRAS inhibitors, one or more autophagy inhibitors, one or more ABC transporter inhibitors, or one or more dual ABC transporter/kinase inhibitors.

In some embodiments, the PROTAC therapeutic agent and the one or more kinase inhibitors, one or more KRAS inhibitors, one or more autophagy inhibitors, one or more ABC transporter inhibitors, or one or more dual ABC transporter/kinase inhibitors are co-administered to the subject together in a single pharmaceutical composition. In some embodiments, the single pharmaceutical composition is an oral dosage form, an intravenous dosage form, a topical dosage form, an intraperitoneal dosage form, or an intrathecal dosage form. In some embodiments, the single pharmaceutical composition is an oral dosage form or an intravenous dosage form. In some embodiments, the single pharmaceutical composition is an oral dosage form. In some embodiments, the single pharmaceutical composition is an intravenous dosage form. In some embodiments, the oral dosage form is a pill, tablet, capsule, gel-cap, or liquid. In some embodiments, the oral dosage form is a pill. In some embodiments, the oral dosage form is a tablet. In some embodiments, the oral dosage form is a capsule. In some embodiments, the oral dosage form is a gel-cap. In some embodiments, the oral dosage form is a liquid.

In some embodiments, the pharmaceutical composition is an oral dosage form, an intravenous dosage form, a topical dosage form, an intraperitoneal dosage form, or an intrathecal dosage form. In some embodiments, the pharmaceutical composition is an oral dosage form or an intravenous dosage form. In some embodiments, the pharmaceutical composition is an oral dosage form.

In some embodiments, the oral dosage form is a pill, tablet, capsule, cachet, gel-cap, pellet, powder, granule, or liquid. In some embodiments, the oral dosage form is a pill, tablet, capsule, gel-cap, or liquid. In some embodiments, the oral dosage form is a pill. In some embodiments, the oral dosage form is a tablet. In some embodiments, the oral dosage form is a capsule. In some embodiments, the oral dosage form is a gel-cap. In some embodiments, the oral dosage form is a liquid.

In some embodiments, the oral dosage form is protected from light and present within a blister pack, bottle, or intravenous bag. In some embodiments, the oral dosage form is present within a blister pack, bottle, or intravenous bag. In some embodiments, the oral dosage form is present within a blister pack. In some embodiments, the oral dosage form is present within a bottle. In some embodiments, the oral dosage form is present within an intravenous bag.

The compounds and compositions described herein can be administered by any route of administration including, but not limited to, oral, intravenous, topical, intraperitoneal, and intrathecal. In some embodiments, the administration is oral, intravenous, intraperitoneal, or intrathecal. In some embodiments, the administration is oral, intravenous, or intraperitoneal. In some embodiments, the administration is oral or intravenous. In some embodiments, the administration is oral or topical. In some embodiments, the administration is oral or intraperitoneal. In some embodiments, the administration is oral or intrathecal. The route of administration can depend on the particular disease, disorder, or condition being treated and can be selected or adjusted by the clinician according to methods known to the clinician to obtain desired clinical responses. Methods for administration are known in the art and one skilled in the art can refer to various pharmacologic references for guidance (see, for example, Modem Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980)).

In some embodiments, it may be desirable to administer one or more compounds, or a pharmaceutically acceptable salt thereof, or composition(s) comprising the same to a particular area in need of treatment. This may be achieved, for example, by local infusion (for example, during surgery), topical application (for example, with a wound dressing after surgery), or by injection (for example, by depot injection). Formulations for injection can be presented in unit dosage form, such as in ampoules or in multi-dose containers, with an added preservative.

The compounds and compositions described herein can be formulated for parenteral administration by injection, such as by bolus injection or continuous infusion. The compounds and compositions can be administered by continuous infusion subcutaneously over a period of about 15 minutes to about 24 hours. The compositions can take such forms as suspensions, solutions or emulsions in oily or aqueous vehicles, and can contain formulatory agents such as suspending, stabilizing and/or dispersing agents. In some embodiments, the injectable is in the form of short-acting, depot, or implant and pellet forms injected subcutaneously or intramuscularly. In some embodiments, the parenteral dosage form is the form of a solution, suspension, emulsion, or dry powder.

For oral administration, the compounds and compositions described herein can be formulated by combining the compounds with pharmaceutically acceptable carriers. Such carriers enable the compounds to be formulated as tablets, pills, dragees, capsules, emulsions, liquids, gels, syrups, caches, pellets, powders, granules, slurries, lozenges, aqueous or oily suspensions, and the like, for oral ingestion by a subject to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations including, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, including, but not limited to, the cross-linked polyvinyl pyrrolidone, agar, or alginic acid or a salt thereof such as sodium alginate.

Orally administered compounds and compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, when in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. Oral compositions can include standard vehicles such as, for example, mannitol, lactose, starch, magnesium stearate, sodium saccharine, cellulose, magnesium carbonate, etc. Such vehicles are suitably of pharmaceutical grade.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added.

In transdermal administration, the compounds and compositions can be applied to a plaster, or can be applied by transdermal, therapeutic systems that are consequently supplied to the organism. In some embodiments, the compounds and compositions are present in creams, solutions, powders, fluid emulsions, fluid suspensions, semi-solids, ointments, pastes, gels, jellies, and foams, or in patches containing any of the same.

The compounds and compositions described herein can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds and compositions can be formulated with suitable polymeric or hydrophobic materials (for example as an emulsion in an acceptable oil) or ion exchange resins, or as sparingly soluble derivatives, for example, as a sparingly soluble salt.

In some embodiments, the compounds and compositions can be delivered in a controlled release system. In some embodiments, a pump may be used (see Langer, supra; Sefton, CRC Crit. Ref. Biomed. Eng., 1987, 14, 201; Buchwald et al., Surgery, 1980, 88, 507 Saudek et al., N. Engl. J. Med., 1989, 321, 574). In some embodiments, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger et al., J. Macromol. Sci. Rev. Macromol. Chem., 1983, 23, 61; see, also Levy et al., Science, 1985, 228, 190; During et al., Ann. Neurol., 1989, 25, 351; Howard et al., J. Neurosurg., 1989, 71, 105). In some embodiments, a controlled-release system can be placed in proximity of the target of the compounds described herein, such as the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, Science, 1990, 249, 1527-1533) may be used.

The compounds and compositions described herein can be contained in formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The pharmaceutical compositions can also comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. In some embodiments, the compounds described herein can be used with agents including, but not limited to, topical analgesics (e.g., lidocaine), barrier devices (e.g., GelClair), or rinses (e.g., Caphosol). Pharmaceutical carriers can be liquids, such as water and oils, including those of petroleum, animal, vegetable or synthetic origin, such as peanut oil, soybean oil, mineral oil, sesame oil, and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

In some embodiments, the compounds and compositions described herein can be delivered in a vesicle, in particular a liposome (see, Langer, Science, 1990, 249, 1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

The compositions described herein can be administered either alone (as a single composition comprising the compounds described herein) or in combination (concurrently or serially) with other pharmaceutical agents. For example, the compounds and compositions can be administered in combination with anti-cancer or anti-neoplastic agents (for example, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel) or therapies (for example, surgery or radiotherapy).

The amount of any particular compound to be administered may be that amount which is therapeutically effective. The dosage to be administered may depend on the characteristics of the subject being treated, e.g., the particular animal treated, age, weight, health, types of concurrent treatment, if any, and frequency of treatments, and on the nature and extent of the disease, condition, or disorder, and can be easily determined by one skilled in the art (e.g., by the clinician). The selection of the specific dose regimen can be selected or adjusted or titrated by the clinician according to methods known to the clinician to obtain the desired clinical response. In addition, in vitro or in vivo assays may optionally be employed to help identify optimal dosage ranges. The precise dose to be employed in the compositions may also depend on the route of administration, and should be decided according to the judgment of the practitioner and each patient's circumstances.

Suitable compositions include, but are not limited to, oral non-absorbed compositions. Suitable compositions also include, but are not limited to saline, water, cyclodextrin solutions, and buffered solutions of pH 3-9.

The compounds and compositions described herein can be formulated with numerous excipients including, but not limited to, purified water, propylene glycol, PEG 400, glycerin, DMA, ethanol, benzyl alcohol, citric acid/sodium citrate (pH3), citric acid/sodium citrate (pH5), tris(hydroxymethyl)amino methane HCl (pH7.0), 0.9% saline, and 1.2% saline, and any combination thereof. In some embodiments, excipient is chosen from propylene glycol, purified water, and glycerin.

In some embodiments, the excipient is a multi-component system chosen from 20% w/v propylene glycol in saline, 30% w/v propylene glycol in saline, 40% w/v propylene glycol in saline, 50% w/v propylene glycol in saline, 15% w/v propylene glycol in purified water, 30% w/v propylene glycol in purified water, 50% w/v propylene glycol in purified water, 30% w/v propylene glycol and 5 w/v ethanol in purified water, 15% w/v glycerin in purified water, 30% w/v glycerin in purified water, 50% w/v glycerin in purified water, 20% w/v Kleptose in purified water, 40% w/v Kleptose in purified water, and 25% w/v Captisol in purified water. In some embodiments, the excipient is chosen from 50% w/v propylene glycol in purified water, 15% w/v glycerin in purified water, 20% w/v Kleptose in purified water, 40% w/v Kleptose in purified water, and 25% w/v Captisol in purified water. In some embodiments, the excipient is chosen from 20% w/v Kleptose in purified water, 20% w/v propylene glycol in purified water, and 15% w/v glycerin in purified water.

In some embodiments, the compounds and compositions described herein can be lyophilized to a solid and reconstituted with, for example, water prior to use.

When administered to a human, the compounds and compositions can be sterile. Water is a suitable carrier when the compound and composition is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The compositions described herein can take the form of a solution, suspension, emulsion, tablet, pill, pellet, capsule, capsule containing a liquid, powder, sustained-release formulation, aerosol, spray, or any other form suitable for use. Examples of suitable pharmaceutical carriers are described in Remington's Pharmaceutical Sciences, A. R. Gennaro (Editor) Mack Publishing Co.

In some embodiments, the compounds and compositions are formulated in accordance with routine procedures as pharmaceutical compositions adapted for administration to humans. Typically, compounds are solutions in sterile isotonic aqueous buffer. Where necessary, the compositions can also include a solubilizing agent. Compositions for intravenous administration may optionally include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound or composition is to be administered by infusion, it can be dispensed, for example, with an infusion bottle containing sterile pharmaceutical grade water or saline. Where the compound or composition is administered by injection, an ampoule of sterile water for injection or saline can be provided so that the ingredients may be mixed prior to administration.

The pharmaceutical compositions can be in unit dosage form. In such form, the composition can be divided into unit doses containing appropriate quantities of the active component. The unit dosage form can be a packaged preparation, the package containing discrete quantities of the preparations, for example, packeted tablets, capsules, and powders in vials or ampules. The unit dosage form can also be a capsule, cachet, or tablet itself, or it can be the appropriate number of any of these packaged forms.

In some embodiments, a composition is in the form of a liquid wherein the active agents are present in solution, in suspension, as an emulsion, or as a solution/suspension. In some embodiments, the liquid composition is in the form of a gel. In other embodiments, the liquid composition is aqueous. In other embodiments, the composition is in the form of an ointment.

In some embodiments, the composition is an in situ gellable aqueous solution, suspension or solution/suspension, comprising about from 0.2% to about 3% or from about 0.5% to about 1% by weight of a gelling polysaccharide, chosen from gellan gum, alginate gum and chitosan, and about 1% to about 50% of a water-soluble film-forming polymer, preferably selected from alkylcelluloses (e.g., methylcellulose, ethylcellulose), hydroxyalkylcelluloses (e.g., hydroxyethylcellulose, hydroxypropyl methylcellulose), hyaluronic acid and salts thereof, chondroitin sulfate and salts thereof, polymers of acrylamide, acrylic acid and polycyanoacrylates, polymers of methyl methacrylate and 2-hydroxyethyl methacrylate, polydextrose, cyclodextrins, polydextrin, maltodextrin, dextran, polydextrose, gelatin, collagen, natural gums (e.g., xanthan, locust bean, acacia, tragacanth and carrageenan gums and agar), polygalacturonic acid derivatives (e.g., pectin), polyvinyl alcohol, polyvinylpyrrolidone and polyethylene glycol. The composition can optionally contain a gel-promoting counterion such as calcium in latent form, for example encapsulated in gelatin.

In some embodiments, the composition is an in situ gellable aqueous solution, suspension or solution/suspension comprising about 0.1% to about 5% of a carrageenan gum, e.g., a carrageenan gum having no more than 2 sulfate groups per repeating disaccharide unit, such as e.g., kappa-carrageenan, having 18-25% ester sulfate by weight, iota-carrageenan, having 25-34% ester sulfate by weight, and mixtures thereof.

Optionally one or more stabilizers can be included in the compositions to enhance chemical stability where required. Suitable stabilizers include, but are not limited to, chelating agents or complexing agents, such as, for example, the calcium complexing agent ethylene diamine tetraacetic acid (EDTA). For example, an appropriate amount of EDTA or a salt thereof, e.g., the disodium salt, can be included in the composition to complex excess calcium ions and prevent gel formation during storage. EDTA or a salt thereof can suitably be included in an amount of about 0.01% to about 0.5%. In those embodiments containing a preservative other than EDTA, the EDTA or a salt thereof, more particularly disodium EDTA, can be present in an amount of about 0.025% to about 0.1% by weight.

The present disclosure also provides combinations of a PROTAC therapeutic agent, or a pharmaceutically acceptable salt thereof, and one or more kinase inhibitors, one or more KRAS inhibitors, or one or more autophagy inhibitors, or any combination thereof, or a pharmaceutically acceptable salt thereof, for use in the manufacture of a medicament for treating cancer. Any of the combinations described herein can be used in the manufacture of a medicament for treating any of the cancers described herein. In some embodiments, the PROTAC therapeutic agent is not a BET-PROTAC therapeutic agent or a CDK9-PROTAC therapeutic agent.

The present disclosure also provides combinations of: i) a PROTAC therapeutic agent, or a pharmaceutically acceptable salt thereof, and ii) one or more kinase inhibitors, one or more ABC transporter inhibitors, or one or more dual ABC transporter/kinase inhibitors, or any combination thereof, or a pharmaceutically acceptable salt thereof, for use in the manufacture of a medicament for treating cancer. The present disclosure also provides combinations of a PROTAC therapeutic agent, or a pharmaceutically acceptable salt thereof, and one or more dual ABC transporter/kinase inhibitors, or a pharmaceutically acceptable salt thereof, for use in the manufacture of a medicament for treating cancer.

The present disclosure also provides uses of a pharmaceutical composition comprising a PROTAC therapeutic agent, or a pharmaceutically acceptable salt thereof, and one or more KRAS inhibitors, or one or more autophagy inhibitors, or any combination thereof, or a pharmaceutically acceptable salt thereof, for treating cancer. Any of the combinations described herein can be used for treating any of the cancers described herein. In some embodiments, the PROTAC therapeutic agent is not a BET-PROTAC therapeutic agent or a CDK9-PROTAC therapeutic agent.

The present disclosure also provides uses of a pharmaceutical composition comprising i) a PROTAC therapeutic agent, or a pharmaceutically acceptable salt thereof, and ii) one or more kinase inhibitors, one or more ABC transporter inhibitors, or one or more dual ABC transporter/kinase inhibitors, or any combination thereof, or a pharmaceutically acceptable salt thereof, or a pharmaceutically acceptable salt thereof, for treating cancer. The present disclosure also provides uses of a pharmaceutical composition comprising a PROTAC therapeutic agent, or a pharmaceutically acceptable salt thereof, and one or more dual ABC transporter/kinase inhibitors, or a pharmaceutically acceptable salt thereof, or a pharmaceutically acceptable salt thereof, for treating cancer. Any of the combinations described herein can be used for treating any of the cancers described herein.

In any of the uses described herein, a PROTAC therapeutic agent is linked to a kinase inhibitor in the form of a caged molecule. In any of the uses described herein, a PROTAC therapeutic agent is linked to an ABC transporter inhibitor in the form of a caged molecule. In any of the uses described herein, a PROTAC therapeutic agent is linked to a dual ABC transporter/kinase inhibitor in the form of a caged molecule.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1: General Methodology

### Cell Lines

Cell lines were verified by IDEXX laboratories and free of mycoplasma. CAKI-1, DLD-1, HCT-15, HCT-116, NCI-H747, SW620, SW837, SW948, SW1116, and SW1463 cell lines were maintained in RPMI-1640 supplemented with 10% FBS, 100 U/ml Penicillin-Streptomycin and 2 mM GlutaMAX. A1847, SUM159, and OVCAR3 cell lines were maintained in RPMI-1640 supplemented with 10% FBS, 100 U/ml Penicillin-Streptomycin, 2 mM GlutaMAX, and 5 μg/mL insulin. LS513, and LS1034 cells were maintained in RPMI-1640 supplemented with 10% FBS, 100 U/ml Penicillin-Streptomycin, 2 mM GlutaMAX, 1 mM Sodium Pyruvate and 10 mM HEPES. SK-CO-1 cells were maintained in MEM supplemented with 10% FBS, 100 U/ml Penicillin-Streptomycin, 2 mM GlutaMAX and 1 mM Sodium Pyruvate. PROTAC-resistant cells were maintained with 500 nM PROTAC in the medium. All cells were kept at 37° C. in a 5% CO₂ incubator.

### Compounds

Afatinib, KU-0063794, Lapatinib, MRTX-849, Paclitaxel, PD0325901, and RAD001 were purchased from Selleckchem. JQ1 was purchased from ApexBio. Thal SNS 032, MZ-1, and Tariquidar were purchased from R&D Systems. FAK-PROTAC-Degrader-1, LC-2 and dBET6 were purchased from MedChemExpress. MEK1/2 degraders MS432 and MS934 were provided by the Jian Jin laboratory, Mount Sinai Center for Therapeutics Discovery, Departments of Pharmacological Sciences and Oncological Sciences, Tisch Cancer Institute, Icahn School of Medicine at Mount Sinai, New York, NY 10029.

### Western Blotting

Samples were harvested in MIB lysis buffer (50 mM HEPES (pH 7.5), 0.5% Triton X-100, 150 mM NaCl, 1 mM EDTA, 1 mM EGTA, 10 mM sodium fluoride, 2.5 mM sodium orthovanadate, 1× protease inhibitor cocktail (Roche), and 1% each of phosphatase inhibitor cocktails 2 and 3 (Sigma)). Particulate was removed by centrifugation of lysates at 21,000 rpm for 15 minutes at 4° C. Lysates were subjected to SDS-PAGE chromatography and transferred to PVDF membranes before western blotting with primary antibodies. For a list of primary antibodies used, see (Excel S9B). Secondary HRP-anti-rabbit and HRP-anti-mouse were obtained from ThermoFisher Scientific. SuperSignal West Pico and Femto Chemiluminescent Substrates (Thermo) were used to visualize blots.

### Growth Assays

For short-term growth assays, 3000-5000 cells were plated per well in 96-well plates and allowed to adhere and equilibrate overnight. Drug was added the following morning and after 120 hours of drug treatment, cell viability was assessed using the CellTiter-Glo Luminescent cell viability assay according to manufacturer (Promega). Students t tests were performed for statistical analyses and p values ≤0.05 were considered significant. For long term colony formation assays, cells were plated in 24-well dishes (1000-5000 cells per well) and incubated overnight before continuous drug treatment for 2 weeks, with drug and medium replenished twice weekly. Following the final treatment, cells were rinsed with PBS and fixed with chilled methanol for 10 minutes at −20° C. Methanol was removed by aspiration, and cells were stained with 0.5% crystal violet in 20% methanol for 1 hour at room temperature.

qRT-PCR

GeneJET RNA purification kit (Thermo Scientific) was used to isolate RNA from cells according to manufacturer's instructions. qRT-PCR on diluted cDNA was performed with inventoried TaqMan® Gene Expression Assays on the Applied Biosystems 7500 Fast Real-Time PCR System. The TaqMan Gene Expression Assay probes (ThermoFisher Scientific) used to assess changes in gene expression include ABCB1 (Assay ID: Hsxxxx), and ACTB (control) (Assay ID: Hsxxxx).

### RNAi Knockdown Studies

siRNA transfections were performed using 25 nM siRNA duplex and the reverse transfection protocol. 3000-5000 cells per well were added to 96 well plates with media containing the siRNA and transfection reagent (Lipofectamine RNAiMax) according to the manufacturer's instructions. Cells were allowed to grow for 120 hours post-transfection prior to CellTiter Glo (Promega) analysis. Two-to-three independent experiments were performed with each cell line and siRNA. Students t tests were performed for statistical analyses and p values ≤0.05 were considered significant. For western blot studies, the same procedure was performed with volumes and cell numbers proportionally scaled to a 60 mm or 10 cm dish, and cells were collected 72 hours post-transfection. siRNA product numbers and manufacturers are listed in (Excel S9D).

### Drug Synergy Analysis

Drug synergy was determined using SynergyFinder using the Bliss model and viability as the readout (see, world wide web at “doi.org/10.1093/nar/gkaa216”). Each drug combination was tested in triplicate.

### Immunofluorescence

Cells were plated in a six-well plate with an 18-mm² glass coverslip inside each well. Cells were fixed with 4% paraformaldehyde, permeabilized with 0.1% Triton X-100, blocked with 5% goat serum, and incubated with primary antibody (1:1000, anti-MDR1, Cell Signaling Technology) overnight at 4° C. The slides were washed with PBS and treated with secondary antibody (1:1000, FITC AffiniPure Donkey Anti-Rabbit IgG, Jackson Immunoresearch) for 1 hour at room temperature. Following antibody incubation, coverslips were mounted on slides using ProLong Gold Antifade Reagent with DAPI (4′,6-diamidino-2-phenylindole) (Thermo Fisher Scientific) and allowed to set overnight. Images were taken with a Nikon NI-U fluorescent microscope at 40× magnification.

### Rhodamine 123 Efflux Assay

Efflux assay was performed according to manufacturer's protocol (Millipore Sigma #ECM910). Cells were resuspended in cold efflux buffer and incubated with Rhodamine 123 for 1 hour on ice. Cells were centrifuged and treated in warm efflux buffer with DMSO or drug for 30-60 minutes, washed with cold PBS, and effluxed dye was quantified with a plate reader at an excitation wavelength of 485 nm and an emission wavelength of 530 nm.

### Single Run Total Proteomics

To determine changes in cells chronically exposed to BET or CDK9 PROTACs, we performed single-run proteome analysis as previously described (Lai et al., Front. Oncol., 2020, 10, 2336). Briefly, WT or PROTAC-R cells were lysed in 50 mM HEPES pH 8.0+4% SDS, and 100 μg of proteins were digested using LysC and trypsin. Digested peptides were isolated using C-18 and PGC columns, then dried and cleaned with ethyl acetate. Three pg peptides were analyzed using LC-MS/MS. MaxQuant normalized LFQ values were imported into Perseus software (1.6.2.3) and filtered in the following manner: kinases identified by site only were removed, reverse, or potential contaminant were removed then filtered for kinases identified by >1 unique peptide. Protein LFQ values were log 2 transformed, filtered for a minimum valid number of 3, normalized using Z-scores, annotated, and subjected to a Student's t-test with comparing SPHINX31 vs. DMSO in serum-competent or starved SPEC-2 cells. Parameters for the Student's t-test were the following: S0=0.1, side both using Benjamini-Hochberg FDR <0.05. Volcano plots depicting differences in protein abundance were generated using R studio software. Proteins induced or repressed upon chronic PROTAC exposure (FDR<0.05) were imported into Metascape for pathway analysis (Ansbro et al., PLoS One, 2013, 8, e60334-e60334).

### Nano-LC-MS/MS

Proteolytic peptides were resuspended in 0.1% formic acid and separated with a Thermo Scientific RSLC nano Ultimate 3000 LC on a Thermo Scientific Easy-Spray C18 PepMap 75 μm×50 cm C-18 2 μm column. A 305 minute gradient of 2-20% (180 minutes) 20%-28% (45 minutes) 28%-48% (20 minutes) acetonitrile with 0.1% formic acid was run at 300 nL/minute at 50° C. Eluted peptides were analyzed by Thermo Scientific Q Exactive or Q Exactive plus mass spectrometers utilizing a top 15 methodology in which the 15 most intense peptide precursor ions were subjected to fragmentation. The AGC for MS1 was set to 3×10⁶ with a max injection time of 120 ms, the AGC for MS2 ions was set to 1×10⁵ with a max injection time of 150 ms, and the dynamic exclusion was set to 90 seconds.

### Tumor Xenograft Experiment

Animal studies were conducted in accordance with the guidelines set forth by the Institutional Animal Care and Use Committee. 1×10⁶ LS513 cells were prepared in growth factor reduced Matrigel (Corning) 1:1 and injected into the right flank of 6- to 8-weeks old nude mice. Treatment with MS934 (50 mg/kg), Lapatinib (100 mg/kg) or the combination (using the same dose as monotherapies) were started when tumors reached approximately 150 mm³ and maintained for two weeks. For in vivo studies, MS934 was resuspended in 5% N-methyl-2-pyrrolidinone (NMP), 5% Kolliphor HS-15 (sigma) and 90% saline; and delivered by intraperitoneal injection (daily). Lapatinib was resuspended in 0.5% hydroxypropyl methylcellulose (Sigma) and 0.2% Tween-80 in distilled water pH 8.0; and delivered by oral gavage (daily). Tumor volumes were evaluated every two days using a caliper and the volume was calculated applying the following formula: [(width)²×(length)]/2.

### Proteomics Data Processing

Raw data analysis of LFQ experiments was performed using MaxQuant software 1.6.1.0 and searched using Andromeda 1.5.6.0 against the Swiss-Prot human protein database (downloaded on Apr. 24, 2019, 20402 entries). The search was set up for full tryptic peptides with a maximum of two missed cleavage sites. All settings were default and searched using acetylation of protein N-terminus and oxidized methionine as variable modifications. Carbamidomethylation of cysteine was set as fixed modification. The precursor mass tolerance threshold was set at 10 ppm and maximum fragment mass error was 0.02 Da. LFQ quantitation was performed using MaxQuant with the following parameters; LFQ minimum ratio count: Global parameters for protein quantitation were as follows: label minimum ratio count: 1, peptides used for quantitation: unique, only use modified proteins selected and with normalized average ratio estimation selected. Match between runs was employed for LFQ quantitation and the significance threshold of the ion score was calculated based on a false discovery rate of <1%.

### Example 2: Proteomics Characterization of Degrader-Resistant Cells Reveals Common Upregulation of the Multidrug Resistance Protein MDR1

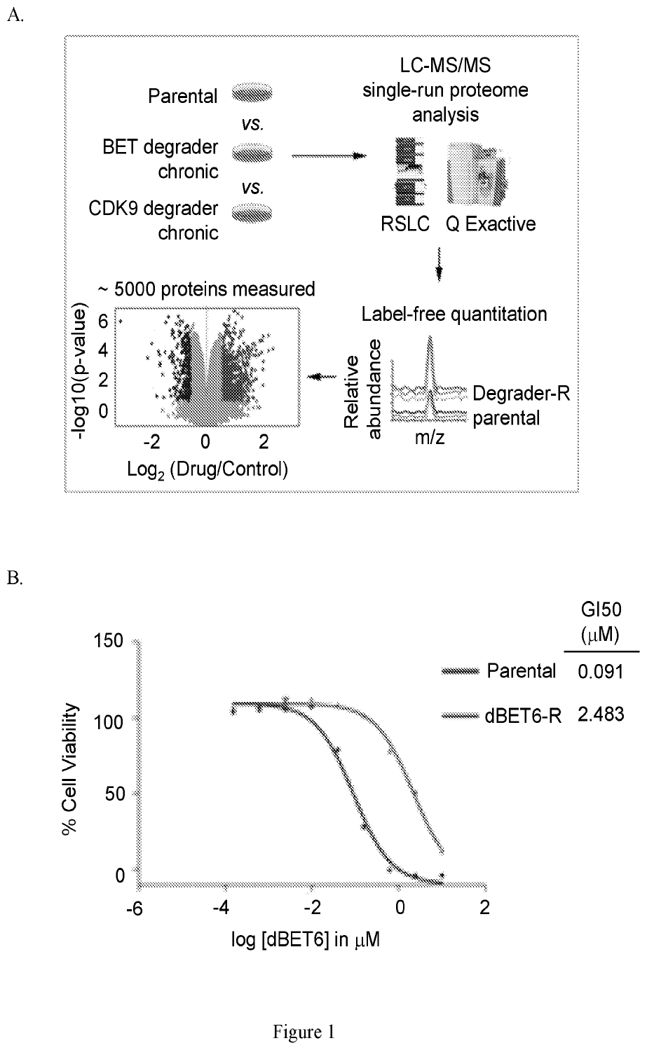

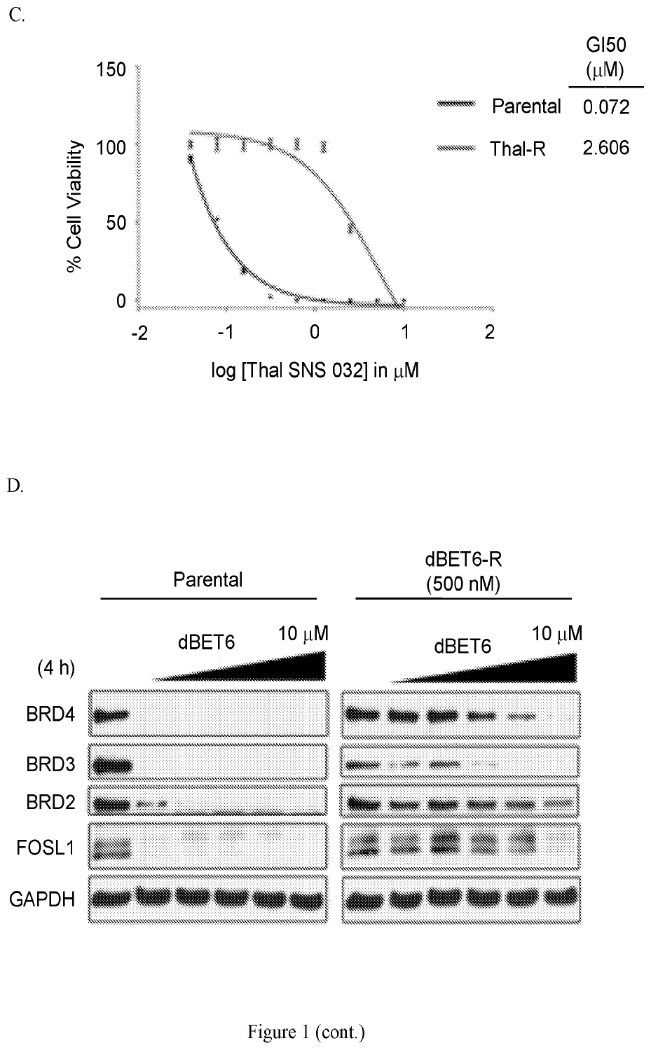

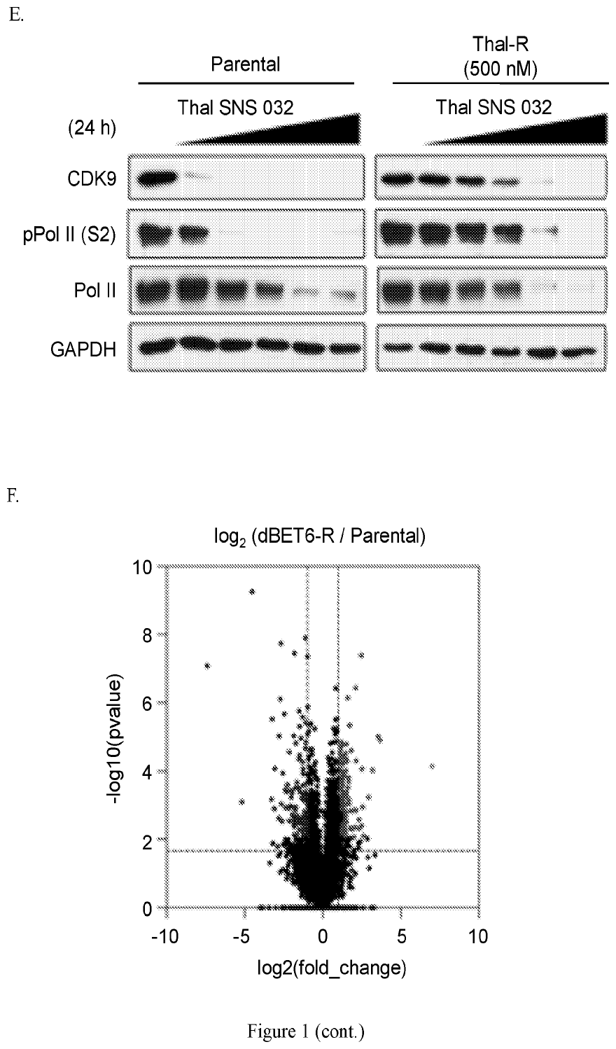

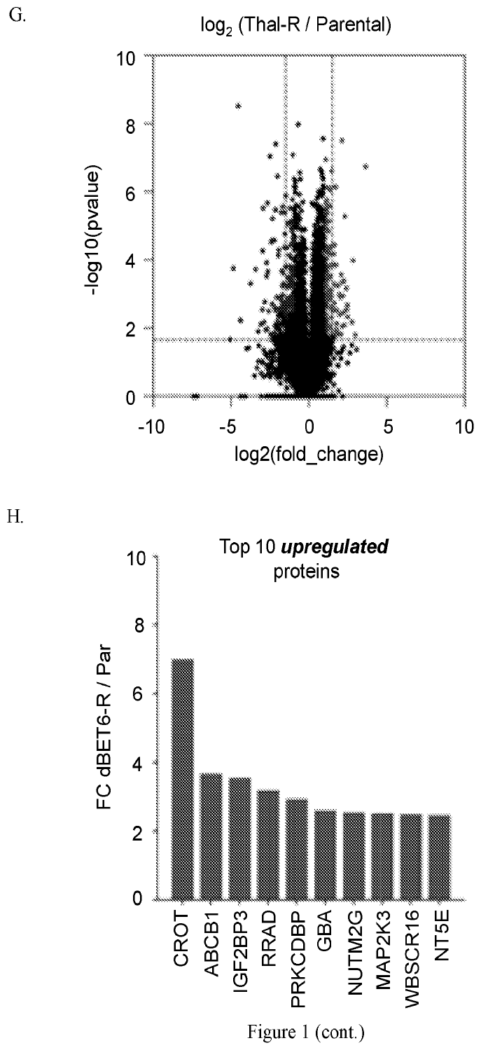

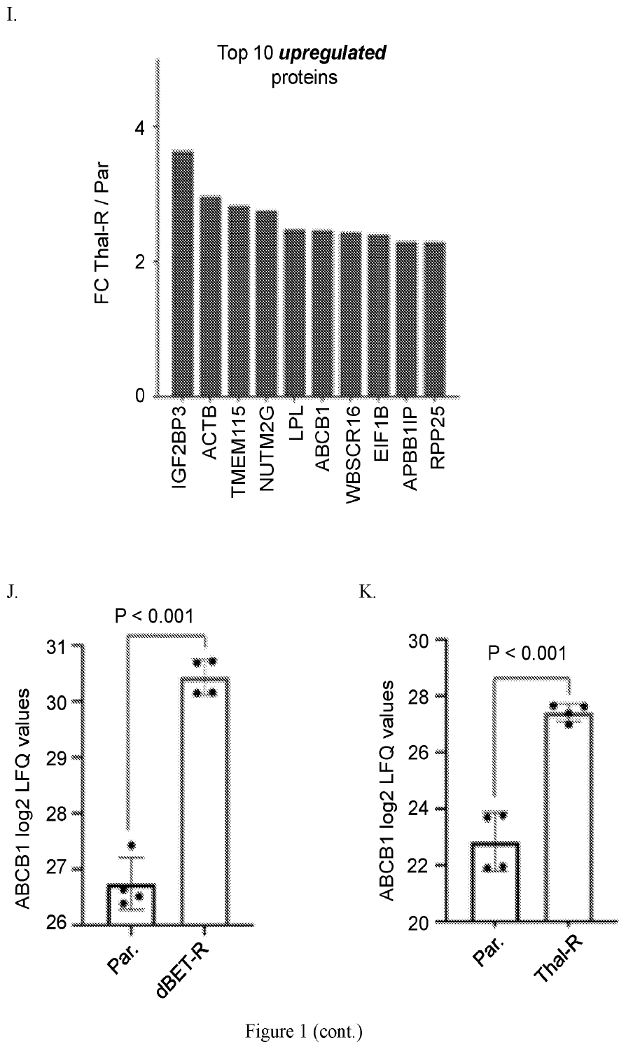

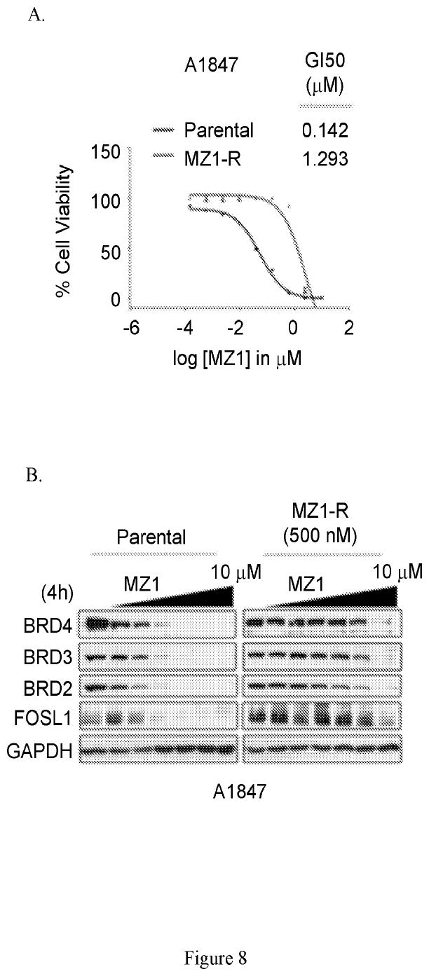

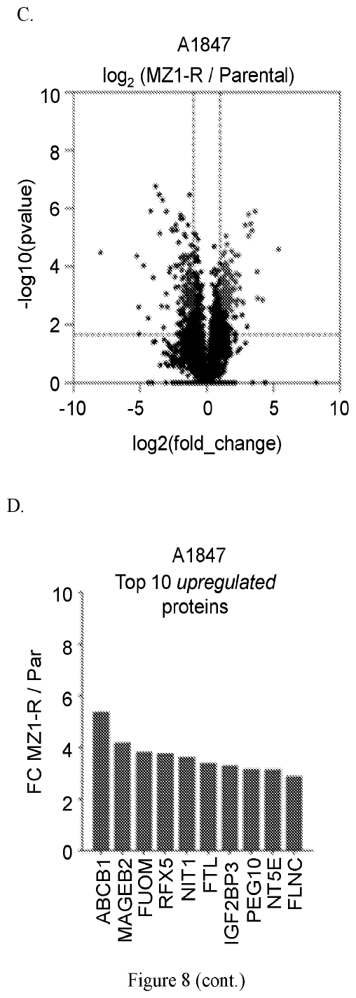

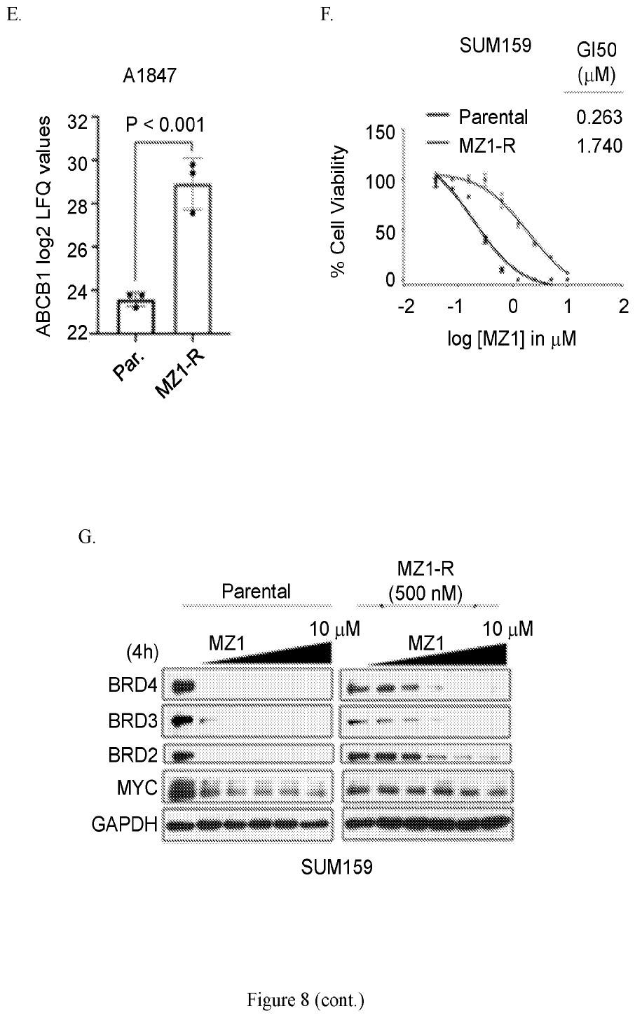

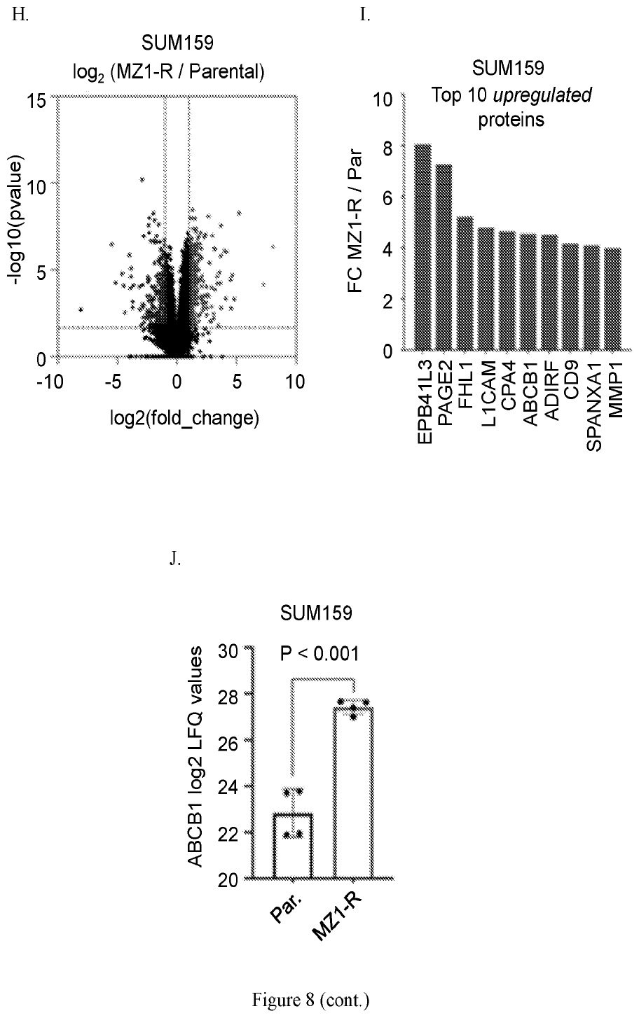

To explore resistance mechanisms to PROTAC therapies, the ovarian cancer cell line A1847 was chronically exposed to BET bromodomain (BD) or CDK9 degraders and single-run proteomics was carried out using LC-MS/MS (Coscia et al., Nature Comm., 2016, 7, 12645) comparing parental and degrader-resistant cells (FIG. 1, Panel A). Changes in protein abundance following chronic degrader-treatment were measured using Label-Free Quantitation (LFQ) (Cox et al., Mol. Cell. Proteomics:MCP, 2014, 13, 2513-2526). A1847 BD or CDK9 degrader-resistant cells were generated through chronic exposure to increasing doses of either dBET6 (Winter et al., Mol. Cell, 2017, 67, 5-18), MZ1 (Zengerle et al., ACS Chem. Biol., 2015, 10, 1770-1777), or Thal SNS 032 (Olson et al., Nat. Chem. Biol., 2018, 14, 163-170). The chronically exposed A1847 cells were more resistant to BET bromodomain or CDK9 degraders than treatment-naïve (i.e., parental) cells, whereby they showed a rightward shift in dose-response cell viability curves (FIG. 1, Panels B and C, and FIG. 8, Panel A). In contrast to parental cells, treatment of chronically exposed cells with increasing doses of BET protein degraders was insufficient to degrade BRD2, BRD3 or BRD4 and reduce BET protein target FOSL1 protein levels to extent observed in parental cells (FIG. 1, Panel D; and FIG. 8, Panel B). Similarly, treatment of A1847 cells with increasing doses of CDK9-degrader Thal SNS 032 did not inhibit cell viability or reduce CDK9 protein levels or CDK9-mediated phosphorylation of RNA polymerase (S2) to the degree observed in parental cells, demonstrating chronic exposure to degraders reduced PROTAC degradation efficiency (FIG. 1, Panel E).

Volcano plot analysis of changes in protein abundance comparing parent and degrader-resistant cells showed significant remodeling of the proteome upon continuous exposure to BET bromodomain or CDK9 degraders (FIG. 1, Panels F and G; FIG. 8, Panel C; Data File Si). A comparison of the top 10 upregulated proteins in BET bromodomain and CDK9 degrader resistant cells revealed 2 proteins were commonly induced, the ATP-dependent drug efflux pump, ATP Binding Cassette Subfamily B Member 1 (ABCB1) (Katayama et al., New J. Science, 2014, 476974), and the RNA binding factor Insulin-Like Growth Factor 2 MRNA-Binding Protein 3 (IGF2BP3) (Mancarella et al., Front. Cell Develop. Biol., 2020, 7, 363) (FIG. 1, Panels H and I; FIG. 8, Panel D). Notably, ABCB1 (MDR1) is a member of the superfamily of ATP-binding cassette (ABC) transporters involved in translocation of drugs and phospholipids across the membrane and has established functions in drug resistance (Fletcher et al., Drug Resistance Updates: Reviews and Commentaries in Antimicrobial and Anticancer Chemotherapy, 2016, 26, 1-9). MDR1 protein levels were upregulated ˜3.5 fold in dBET6-R, ˜5-5-fold in MZ1-R, and ˜2.5-fold in Thal-R cells relative to parental cell lines by LFQ analysis (FIG. 1, Panels J and K; FIG. 8, Panel E). Similarly, chronic exposure of the breast cancer cell line SUM159 with MZ1 resulted in degrader-resistance (FIG. 8, Panels F and G) and proteomics analysis of MZ1-resistant SUM159 cells revealed MDR1 was amongst the top 10 upregulated proteins, with an increase of ˜4.5-fold in MZ1-R cells compared to parental cells (FIG. 8, Panels H, I, and J; Data File S1).

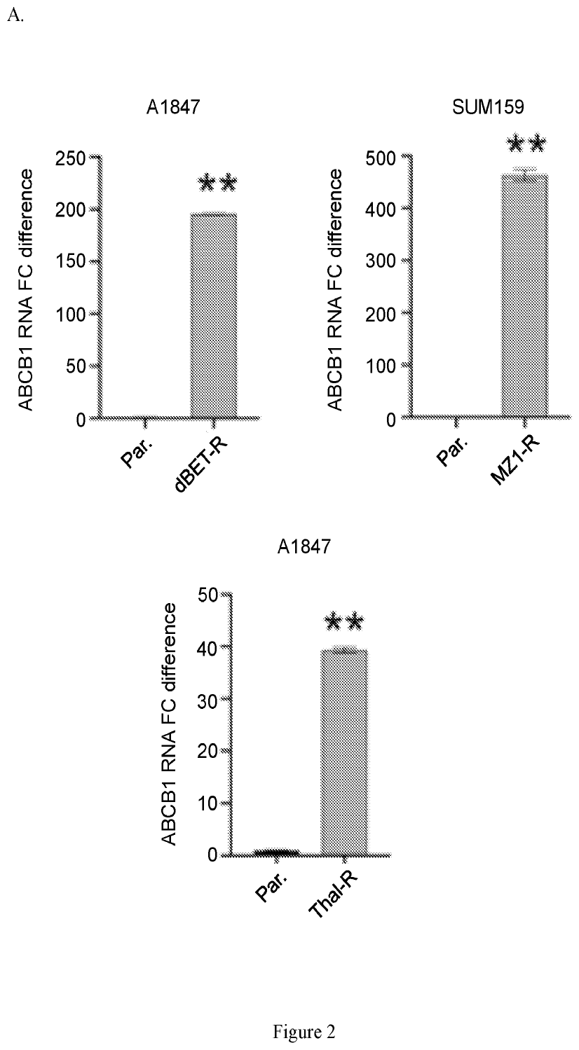

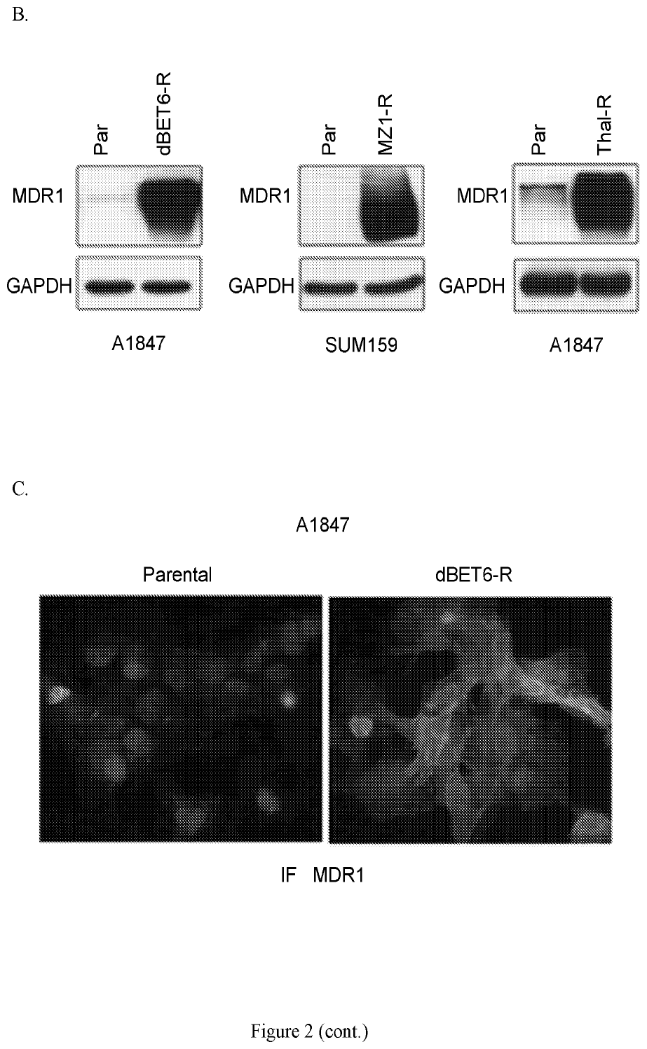

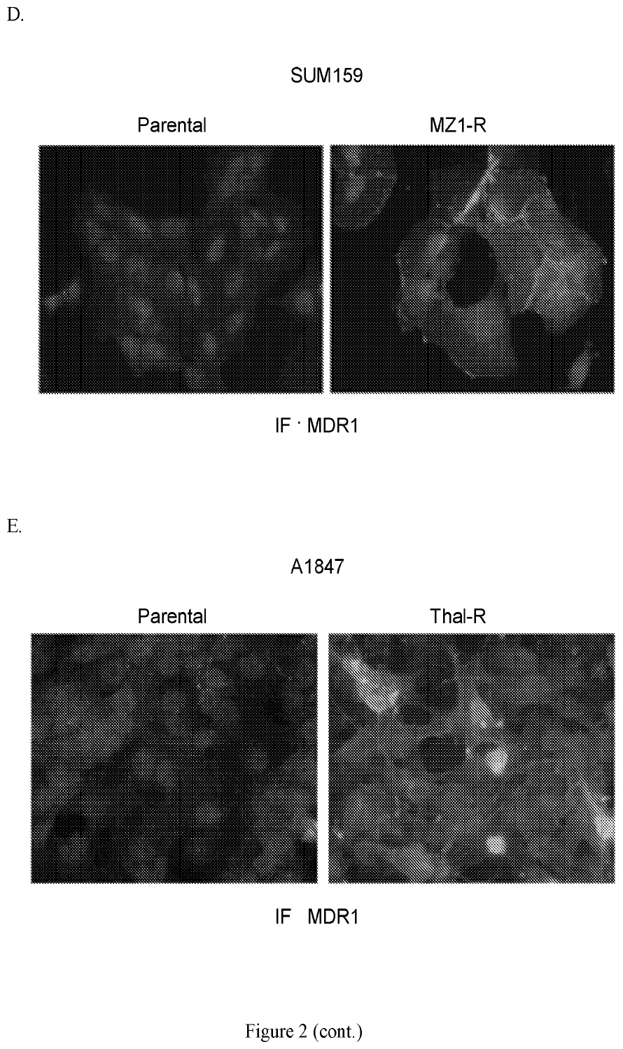

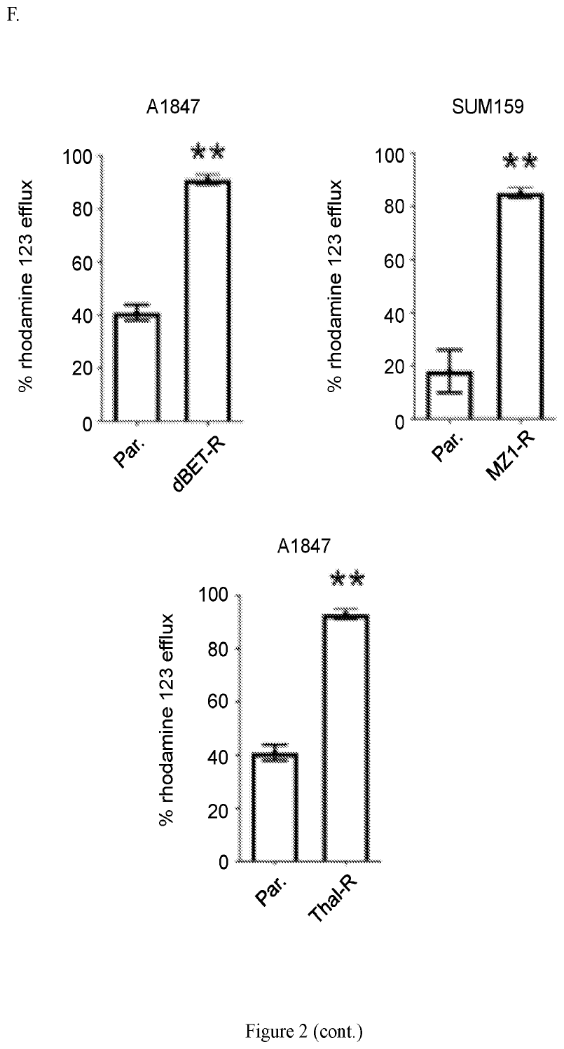

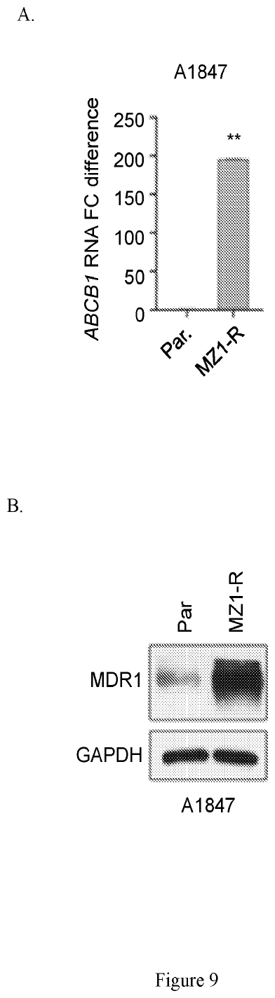

Elevated ABCB1 mRNA and protein levels was confirmed in degrader-resistant A1847 and SUM159 cells by RT-PCR (FIG. 2, Panel A; FIG. 9, Panel A), immunoblot (FIG. 2, Panel B; FIG. 9, Panel B) and immunofluorescence (FIG. 2, Panels C, D, and E), where MDR1 protein was detected at the membrane of degrader-resistant cells. Increased MDR1 drug efflux activity was detected in BET bromodomain or CDK9 degrader-resistant cells relative to parental cells using the Rhodamine 123 efflux assay (Jouan et al., Pharmaceutics, 2016, 8, 12) (FIG. 2, Panel F). Together, these findings demonstrate chronic exposure of cancer cells to BET protein or CDK9 degraders results in increased MDR1 protein levels and drug efflux activity.

### Example 3: Genetic Depletion or Small Molecule Inhibition of MDR1 Re-Sensitizes Degrader-Resistant Cells to PROTACs

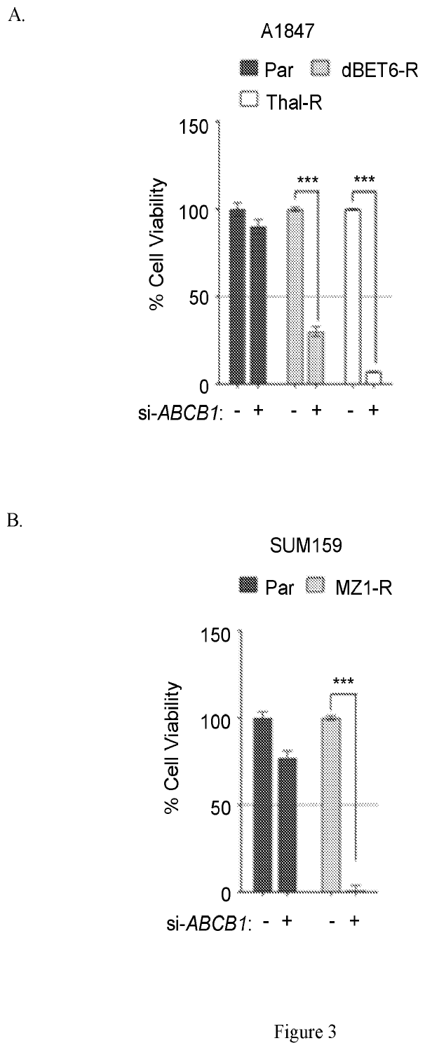

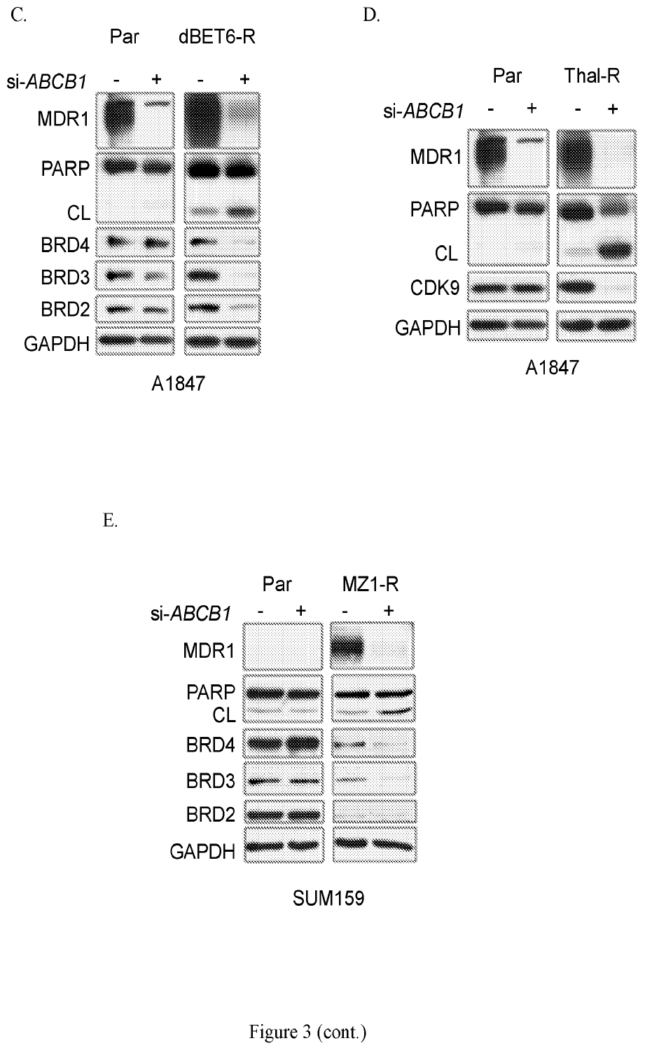

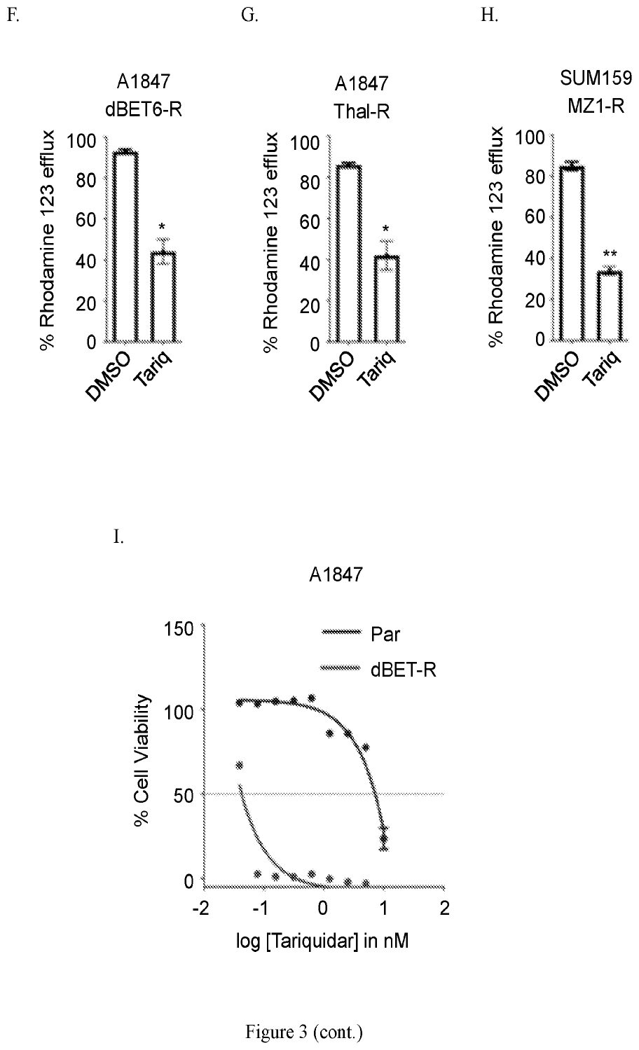

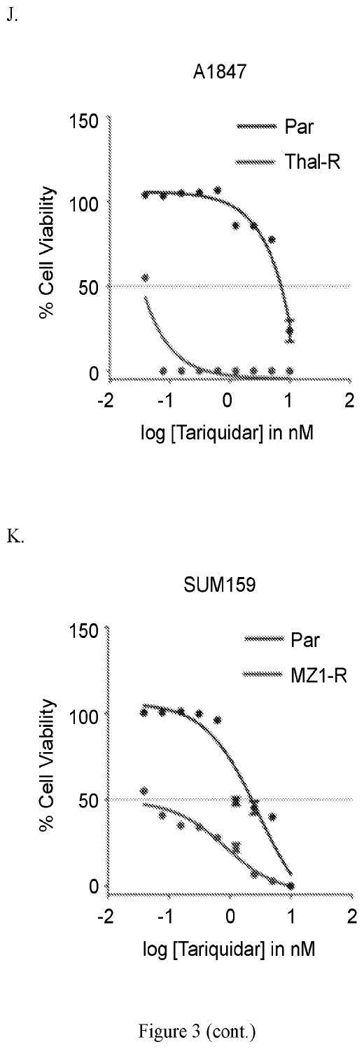

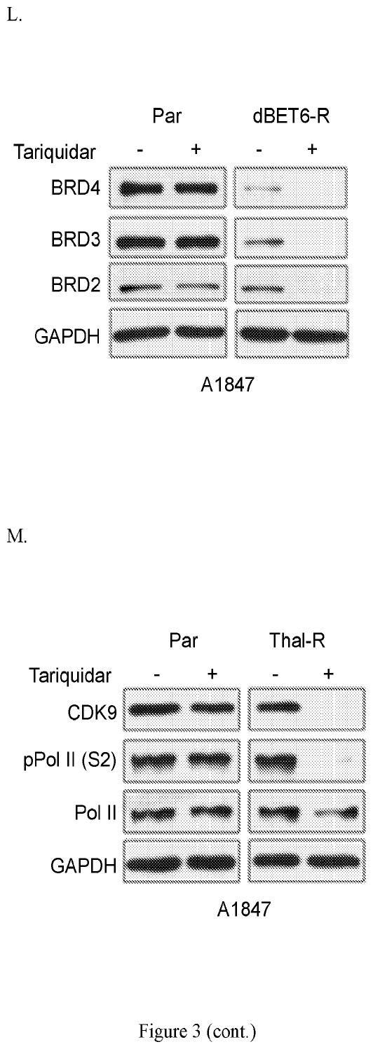

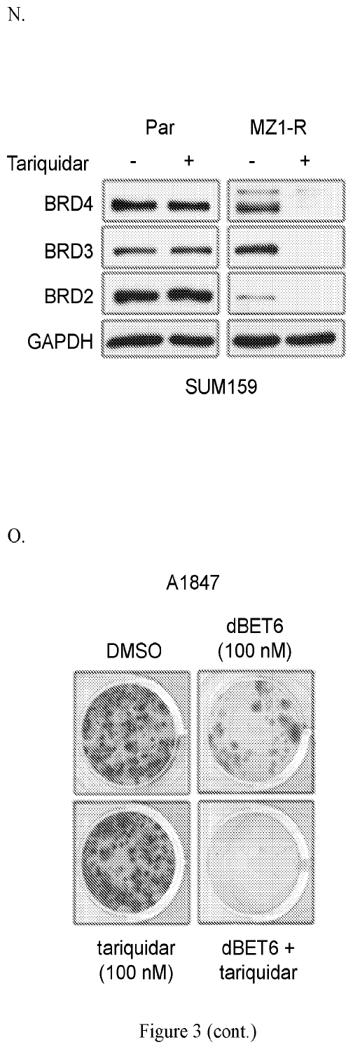

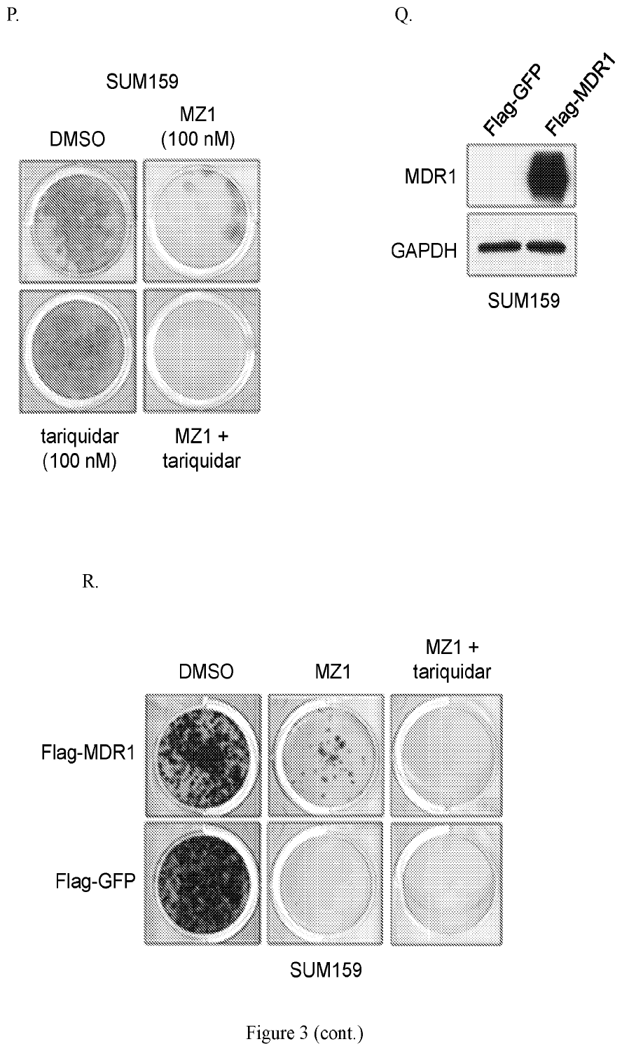

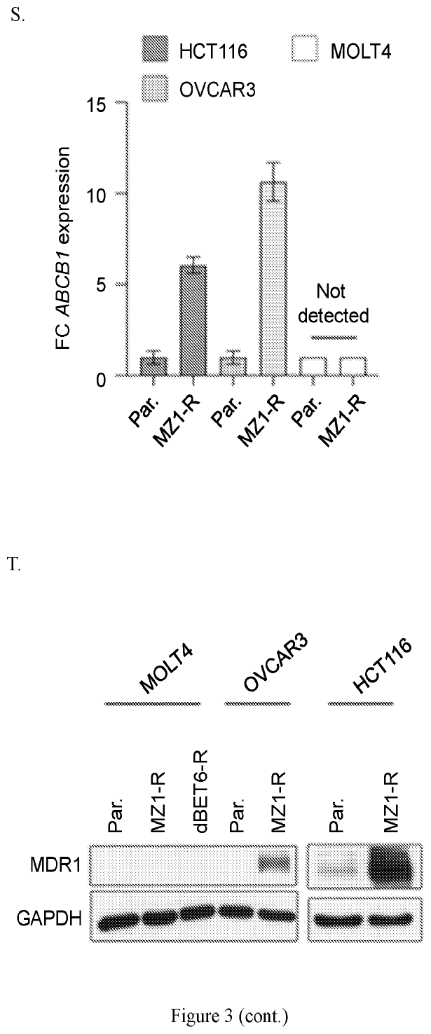

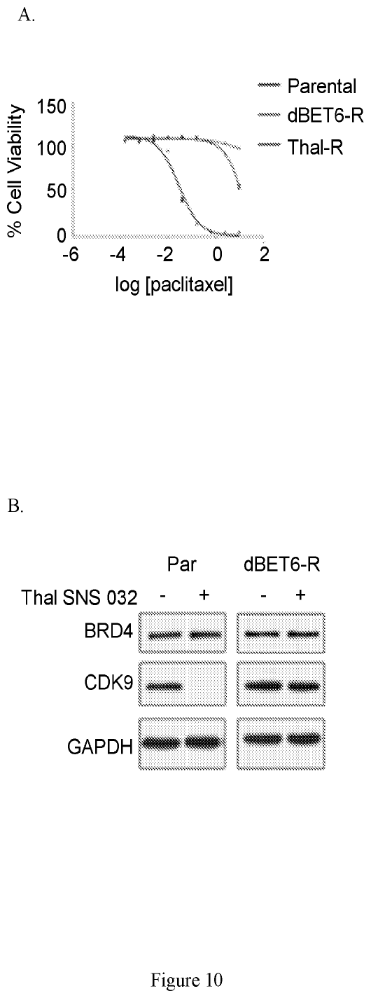

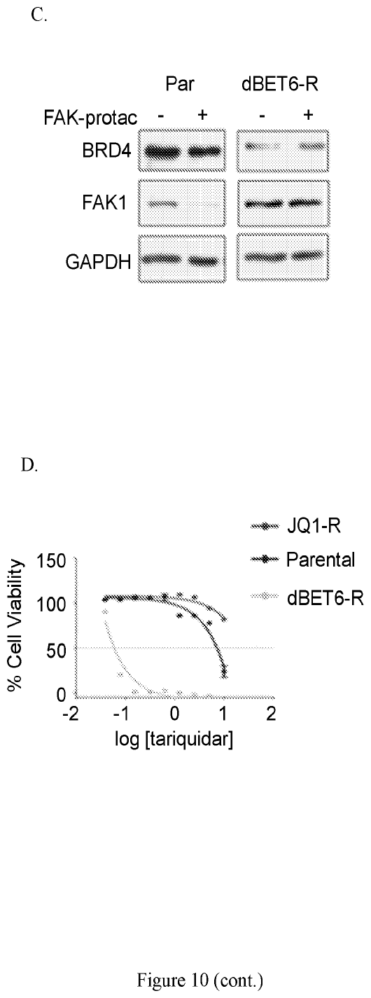

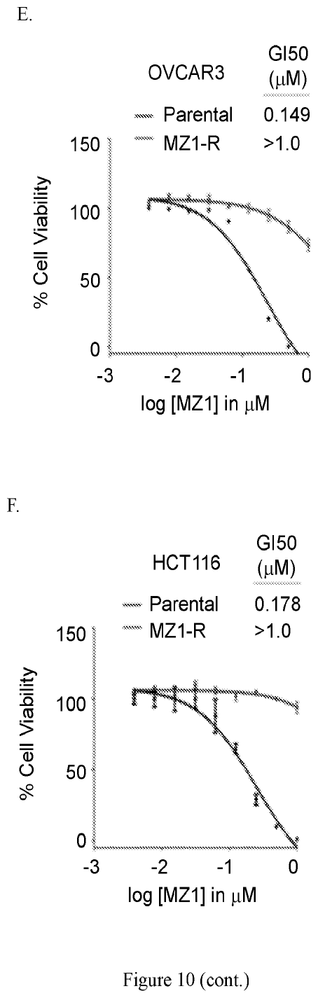

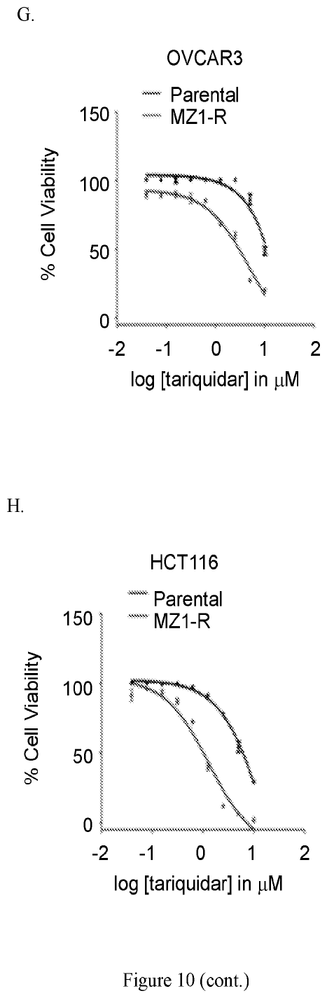

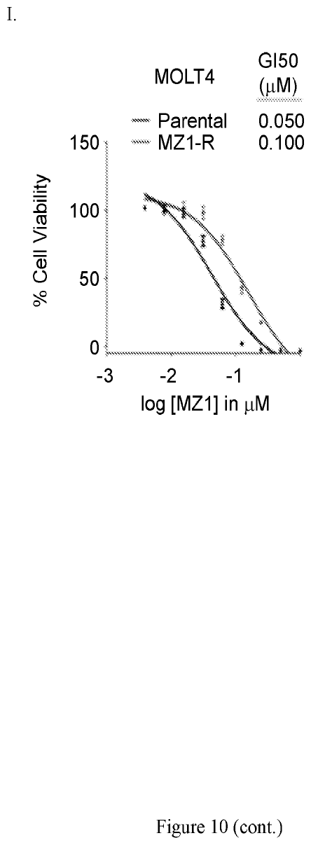

Elevated levels of MDR1 has been shown to promote drug resistance in cancer cells via efflux of large hydrophobic molecules, such as chemotherapy agents (Vaidyanathan et al., Br. J. Cancer, 2016, 115, 431-441). Notably, BET protein or CDK9 degrader-resistant cells acquired resistance to paclitaxel (FIG. 10, Panel A), a known substrate of MDR1 (Vaidyanathan et al., Br. J. Cancer, 2016, 115, 431-441), as well as were cross-resistant to PROTACs targeting other proteins (FIG. 10, Panels B and C). Knockdown of ABCB1 in dBET6-R or Thal-R A1847 cells (FIG. 3, Panel A) or MZ1-R SUM159 cells (FIG. 3, Panel B) reduced cell viability, while parental cells showed minimal effects on cell viability, demonstrating degrader-resistant cells acquired dependency on MDR1 for survival. Moreover, genetic depletion of ABCB1 restored degradation of BET proteins or CDK9 in degrader-resistant cells, re-sensitizing cells to the degraders causing apoptosis (FIG. 3, Panels C, D, and E). In contrast, knockdown of ABCB1 in parental cells showed no effect on BET proteins or CDK9 protein levels nor induced PARP cleavage that was observed in degrader-resistant cells.

Several small molecule inhibitors of MDR1 have been developed, including tariquidar (Weidner et al., Drug Metab. Dispos., 2016, 44, 275-282), which is currently being evaluated in clinical trials for the treatment MDR1-driven drug resistant disease (Pusztai et al., Cancer, 2005, 104, 682-691). Treatment of A1847 dBET6-R, Thal-R or MZ1-R SUM159 cells with tariquidar reduced MDR1 drug efflux pump activity, indicated by reduced efflux of Rhodamine 123 in degrader-resistant cells compared to parental cells (FIG. 3, Panels F, G, and H). Moreover, degrader-resistant cells were more sensitive to tariquidar than parental cells (FIG. 3, Panels I, J, and K), and inhibition of MDR1 function restored degradation of BET proteins or CDK9 (FIG. 3, Panels L, M, and N). Notably, chronic exposure of A1847 cells to BET inhibitor JQ1 did not cause sensitization to tariquidar, suggesting that acquired dependency on MDR1 was distinct to degrader-resistance (FIG. 10, Panel D). Combined treatment of A1847 or SUM159 cells with BET protein degraders and tariquidar blocked the development of BET protein degrader resistant colonies over a 14-day period (FIG. 3, Panels O and P). Moreover, forced expression of Flag-MDR1 in SUM159 cells rescued colony formation growth in MZ1-treated cells that could be blocked by tariquidar-treatment, signifying overexpression of MDR1 reduces sensitivity towards BET degraders (FIG. 3, Panel Q and R).

To further explore MDR1 upregulation in degrader-resistance in cancer cells, 3 additional cancer cell lines (OVCAR3, HCT116, and MOLT4) were chronically exposed to BET protein degraders and assessed MDR1 protein levels. OVCAR3 and HCT116 cell lines acquired resistance to MZ1 (FIG. 10, Panels E and F) that was accompanied by elevated MDR1 mRNA and protein levels in parental cells (FIG. 3, Panels S and T), as well as an increased sensitivity towards tariquidar-treatments (FIG. 10, Panels G and H). In contrast, MZ1-resistant MOLT4 cells (FIG. 10, Panel I) were unable to be generated and chronic exposure to BET protein degraders did not result in upregulation of ABCB1 mRNA or protein levels (FIG. 3, Panels S and T). These findings suggest that not all cancer cells will induce MDR1 following continuous degrader exposure, in these studies, 4 out of 5 cancer cell lines induced MDR1.

Together, these findings demonstrate some cancer cells acquire resistance to degrader therapies through upregulation of the multidrug resistance pump, MDR1 and inhibition of MDR1 restores degrader function overcoming drug resistance in degrader-resistant cancer cells.

### Example 4: MDR1 Overexpressing Cells Exhibit Intrinsic Resistance to PROTAC Therapies that can be Overcome by MDR1 Inhibition

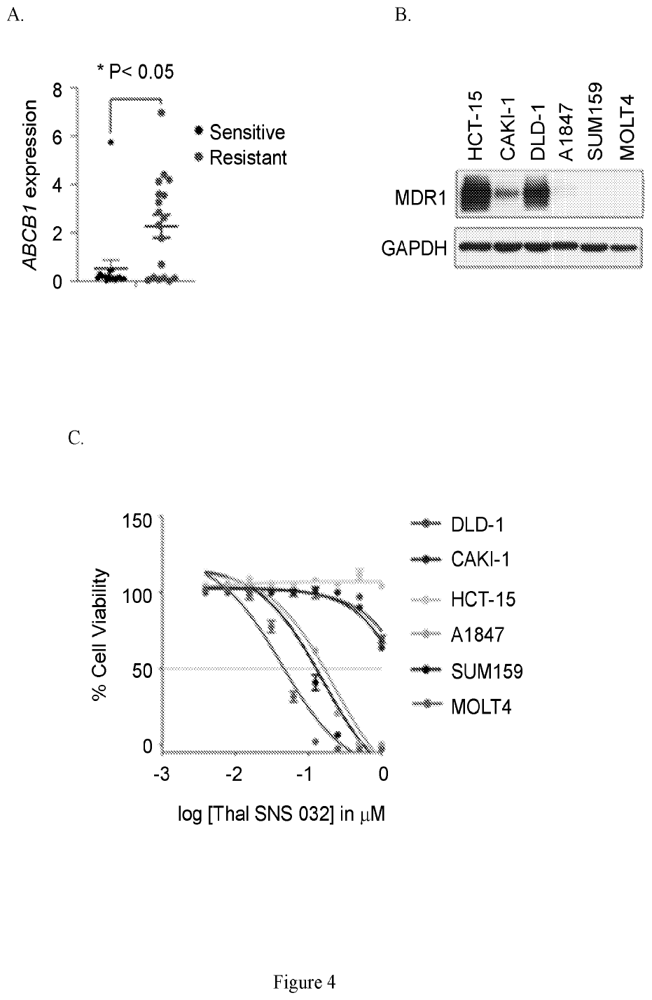

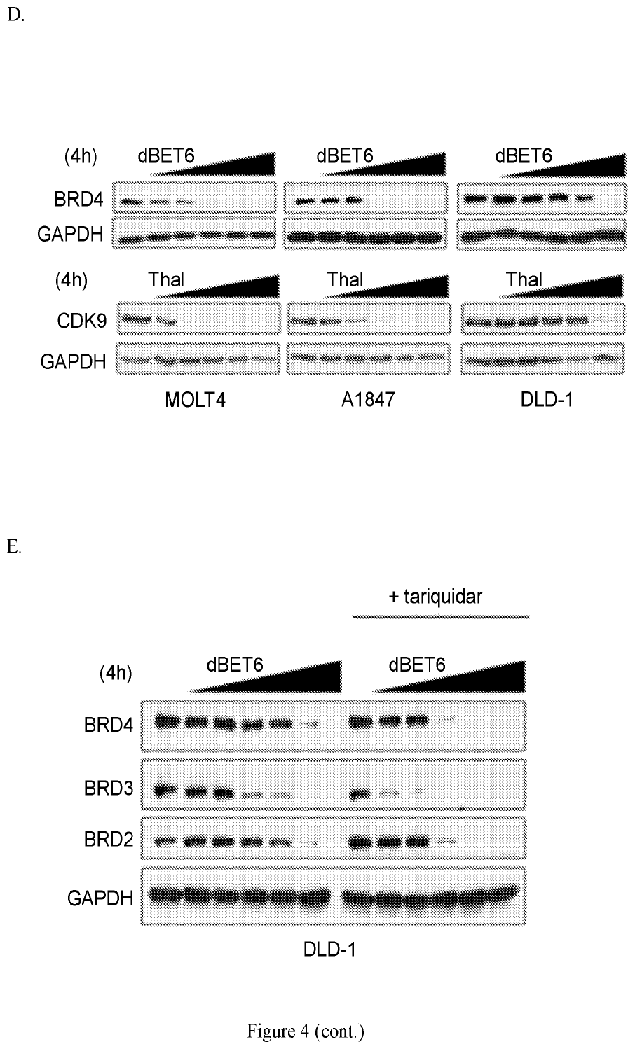

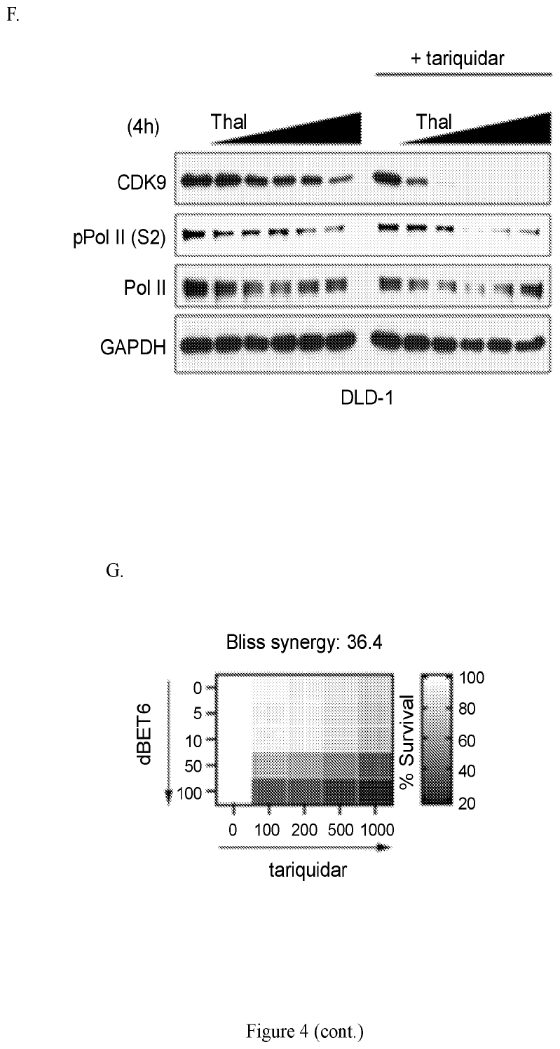

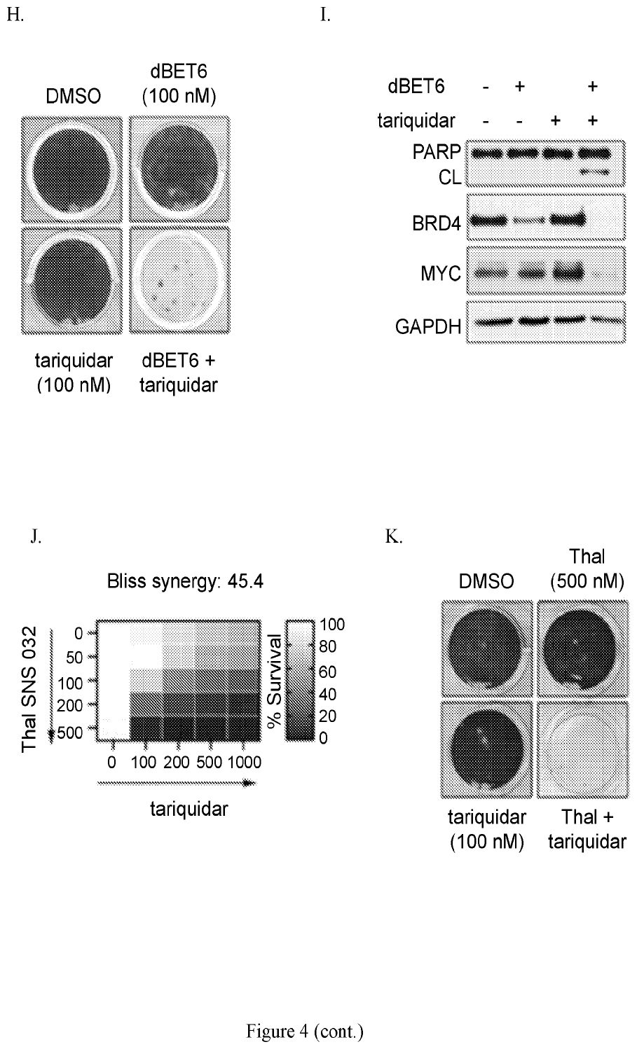

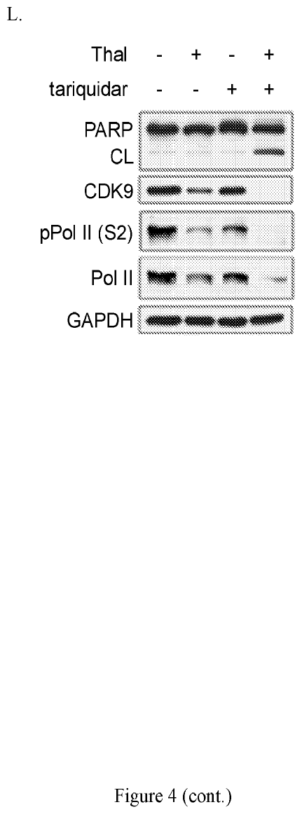

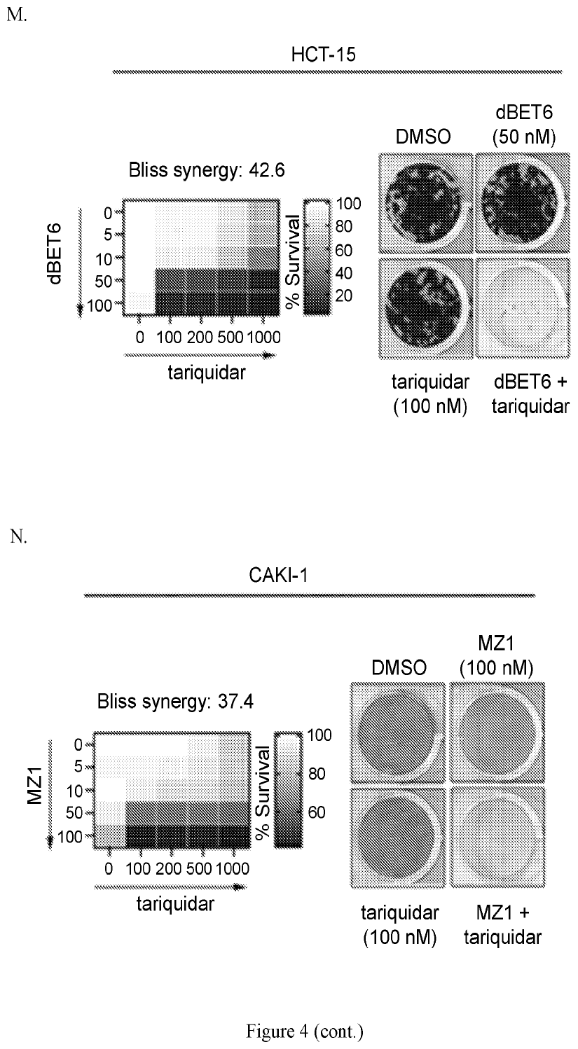

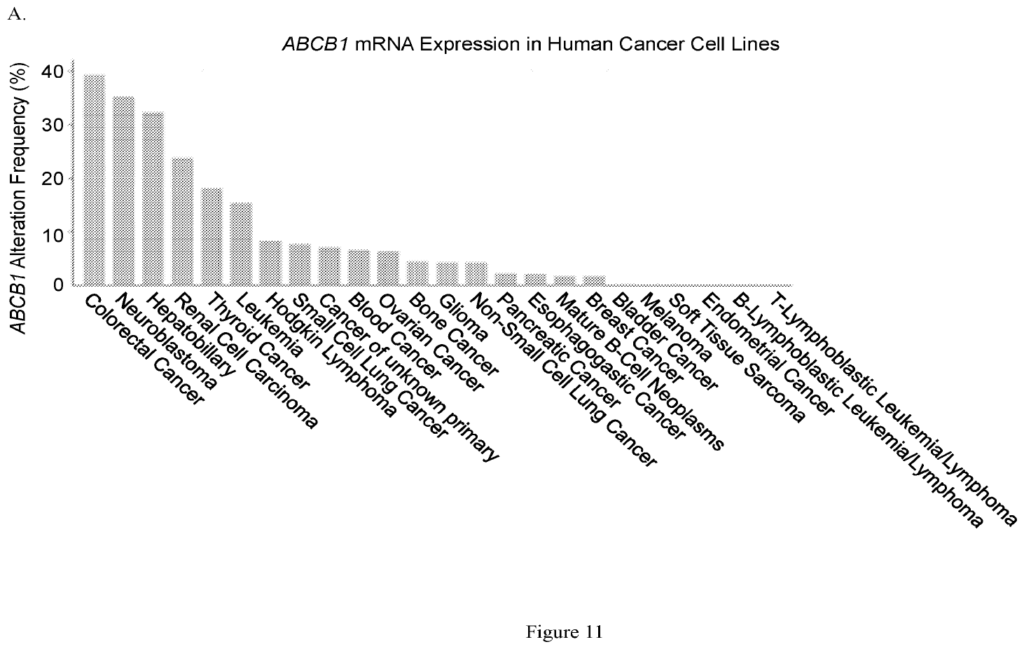

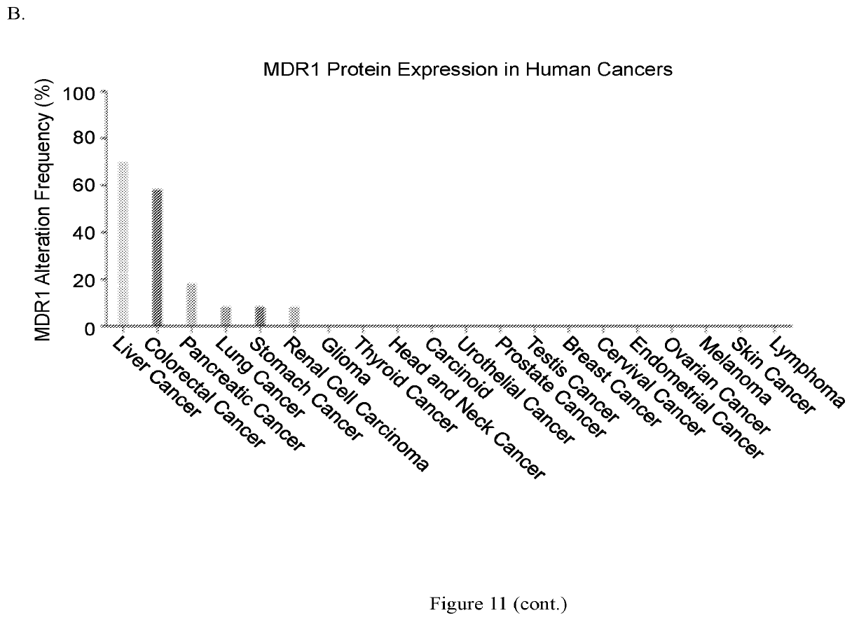

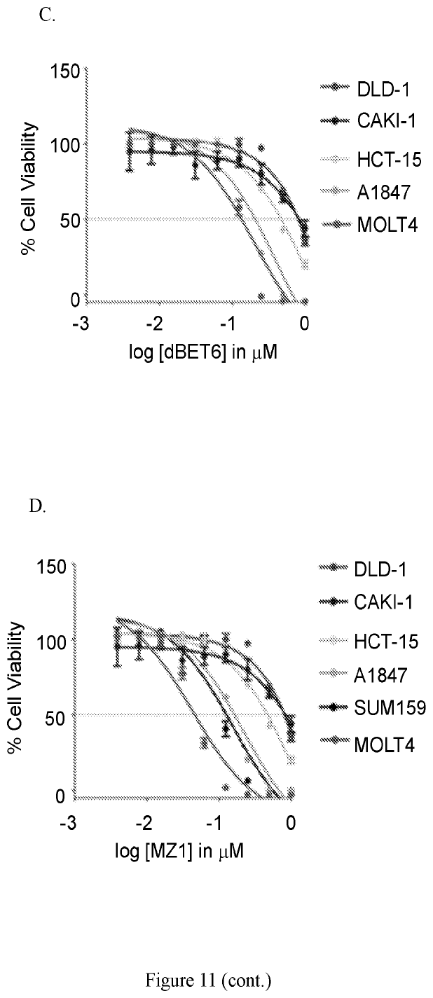

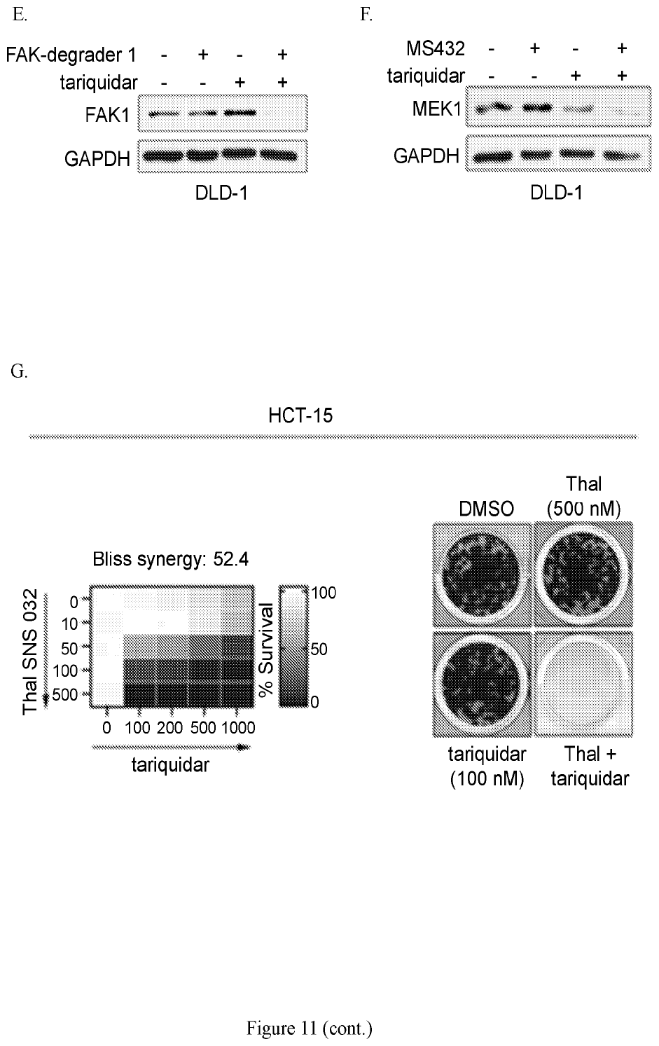

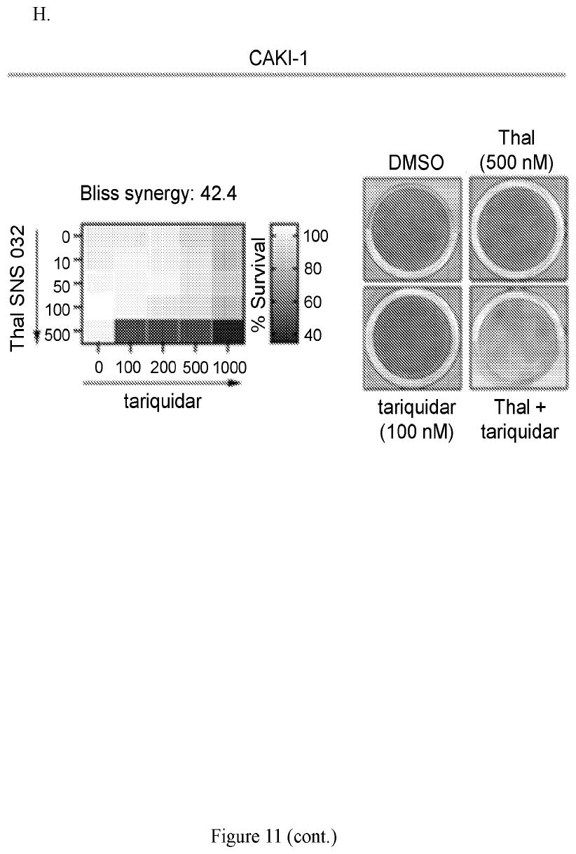

Overexpression of MDR1 frequently occurs in cancers conveying intrinsic resistance to several anti-cancer therapies such as chemotherapies (Katayama et al., New J. Science, 2014, 2014, 476974). Analysis of ABCB1 mRNA expression across the cancer cell line encyclopedia (Cerami et al., Cancer Discov., 2012, 2, 401-404; and Gao et al., Science Signaling, 2013, 6, pl1-pl1) revealed colorectal, neuroblastoma, hepatobiliary and renal cell carcinomas exhibited frequent overexpression of MDR1 (FIG. 11, Panel A). Moreover, querying the human protein atlas, elevated MDR1 protein levels were observed in >50% of liver and colorectal cancer tumors by immunohistochemistry (IHC) (Uhlen et al., Science, 2015, 347, 1260419) (FIG. 11, Panel B). To determine if overexpression of MDR1 in cancer cell lines influences degrader-sensitivity, a prior study was queried which explored MZ1 or dBET6 resistance across a panel of various cancer cell lines (Zhang et al., Mol. Cancer Ther., 2019, 1129.2018) with publicly available ABCB1 mRNA expression datasets (Pharmacogenomic agreement between two cancer cell line data sets, Nature, 2015, 528, 84-87). Notably, cancer cell lines that were resistant to both MZ1 and dBET6 expressed ABCB1 at higher levels than those sensitive to the degraders (P<0.001), suggesting ABCB1 expression represents a biomarker for BET protein degrader response in cancer cells (FIG. 4, Panel A).

To further explore MDR1 as a candidate biomarker for degrader resistance, 3 cancer cell lines, HCT-15 (colon), DLD-1 (colon) and CAKI-1 (renal) with established overexpression of MDR1 were selected and the impact of degrader-treatment on cell viability and protein degradation with cell lines that express low (A1847) or no detectable levels of ABCB1 (SUM159 and MOLT4) was compared by immunoblot (FIG. 4, Panel B). Treatment of MDR1 overexpressing cells with Thal SNS 032, MZ1, or dBET6 did not reduce cell viability to the extent of cancer cell lines expressing low or no detectable MDR1 protein (FIG. 4, Panel C; FIG. 11, Panels C and D). Similarly, treatment of MDR1 overexpressing cell line DLD-1 with dBET6 or Thal SNS 032 did not reduce the intended degrader target to the extent observed with degrader-sensitive A1847 or MOLT4 cells (FIG. 4, Panel D). Importantly, co-treatment of DLD-1 cells with tariquidar and either dBET6 (FIG. 4, Panel E) or Thal SNS 032 (FIG. 4, Panel F) improved the degradation efficiency, resulting in a greater reduction in BET proteins or CDK9 at lower concentrations of the PROTACs. Additionally, co-treatment of DLD-1 cells with FAK degrader (FAK-degrader-1) (Cromm et al., J. Am. Chem. Soc., 2018, 140, 17019-17026) or MEK1/2 degrader (MS432) (Hu et al., J. Med. Chem., 2020, 63, 15883-15905) and tariquidar improved the protein reduction relative to single agent therapies (FIG. 11, Panels E and F), suggesting overexpression of MDR1 promotes resistance to degrader therapies, independent of protein target.

Combination therapies involving BET protein degraders and tariquidar in DLD-1 cells exhibited high drug synergy (Bliss synergy score, 36.4) in blocking cell viability in 5-day growth assays, inhibited colony formation over a 14-day period better than single agent therapies (FIG. 4, Panels G and H). Moreover, co-administration of dBET6 and tariquidar improved protein degradation of BET proteins, reduced BRD4-target MYC and caused apoptosis (FIG. 4, Panel I). Similarly, co-treatment of DLD-1 cells with tariquidar and Thal SNS 032 blocked cell viability, and colony formation to a greater extent than single agent therapies, as well as reduced CDK9 and CDK9-substrate Pol II (S2) and induced apoptosis uniquely in the combination therapy (FIG. 4, Panels J, K, and L). The drug synergy amongst tariquidar and BET protein or CDK9 degraders was also observed in additional MDR1 overexpressing cell lines HCT-15 (FIG. 4, Panel M; FIG. 11, Panel G) and CAKI-1 (FIG. 4, Panel N; FIG. 11, Panel H). Together, our findings suggest specific types of cancers that express high levels of MDR1 such as colorectal or renal cancers will likely exhibit intrinsic resistance to degraders requiring co-administration of MDR1 inhibitors to achieve protein degradation and therapeutic efficacy.

### Example 5: Repurposing Dual Kinase/MDR1 Inhibitors to Overcome Degrader-Resistance in Cancer Cells

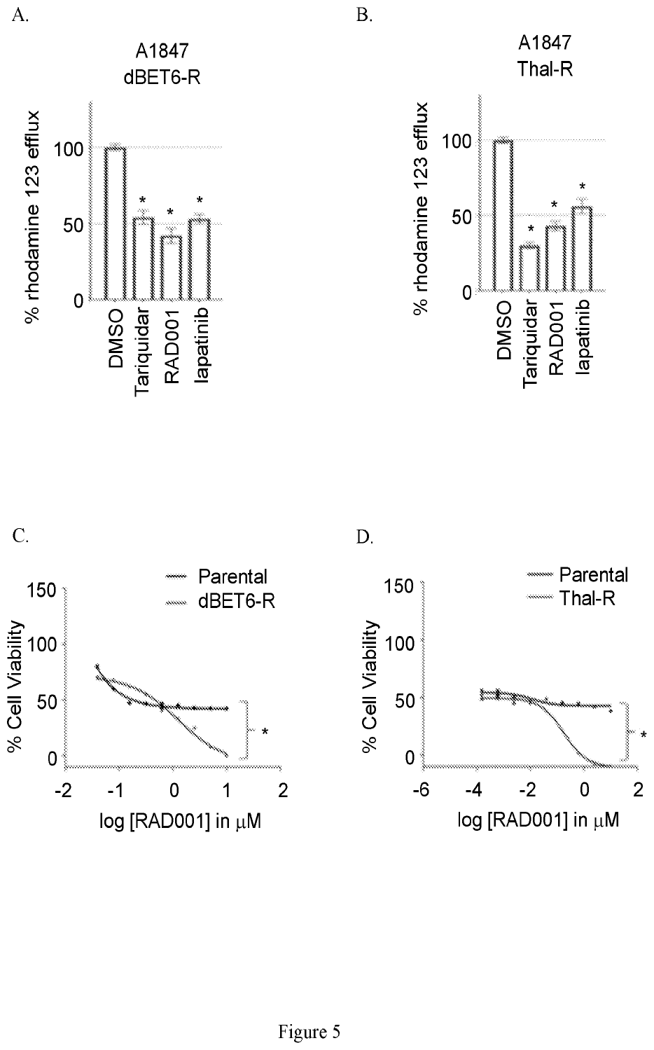

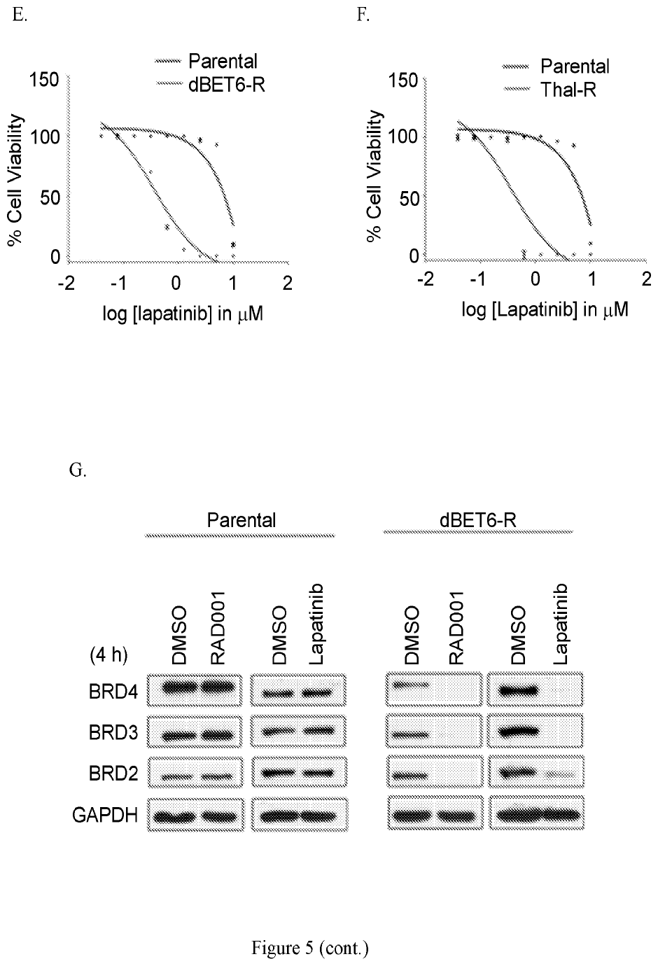

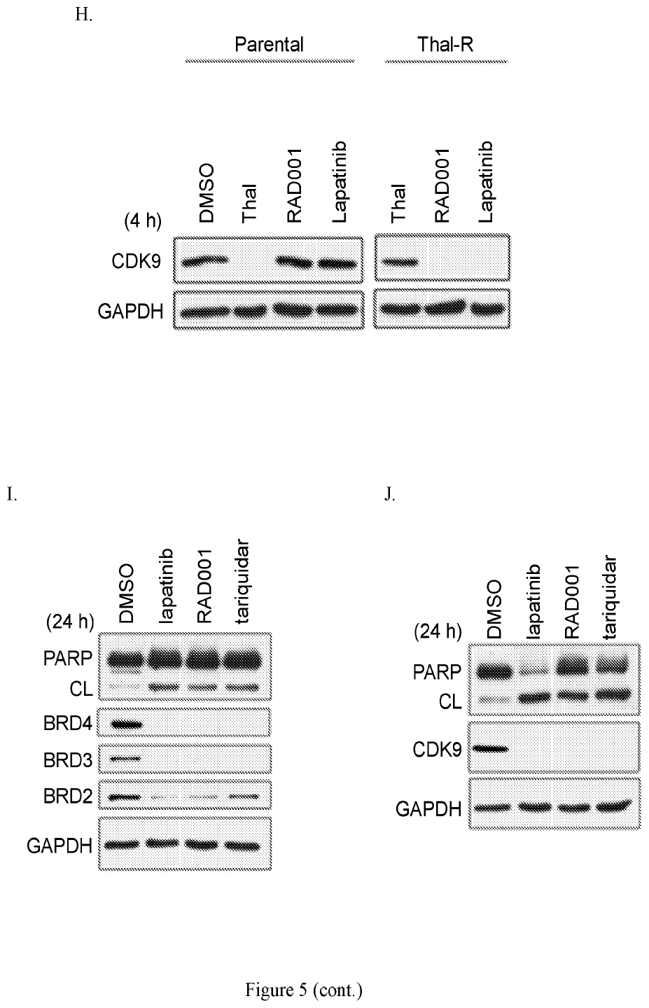

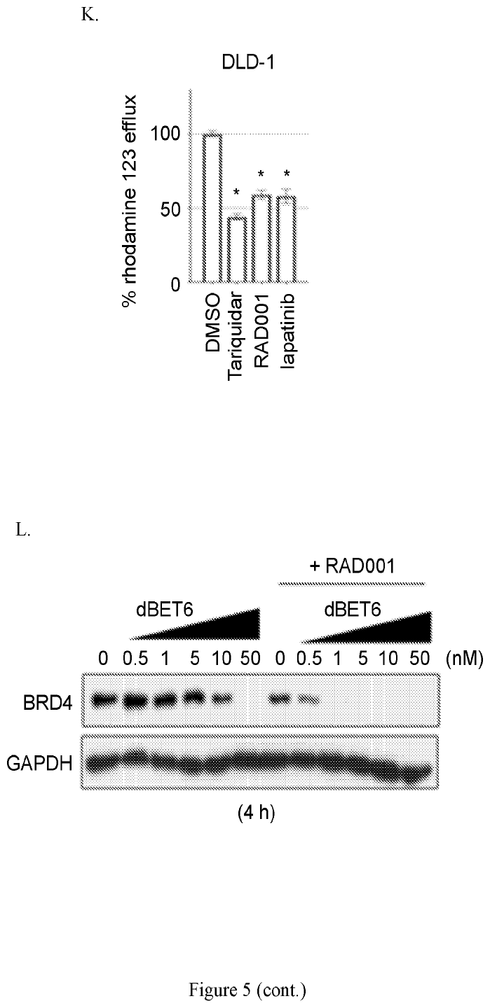

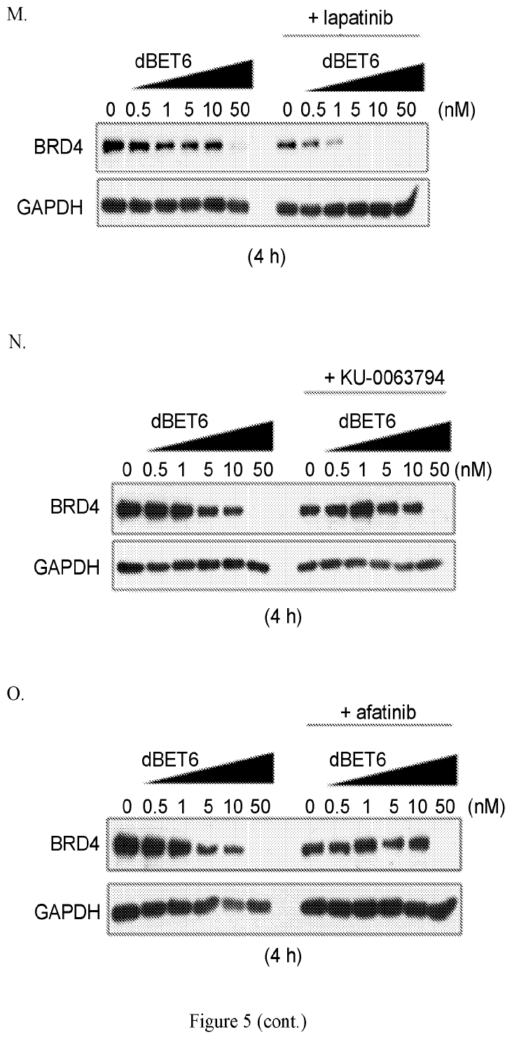

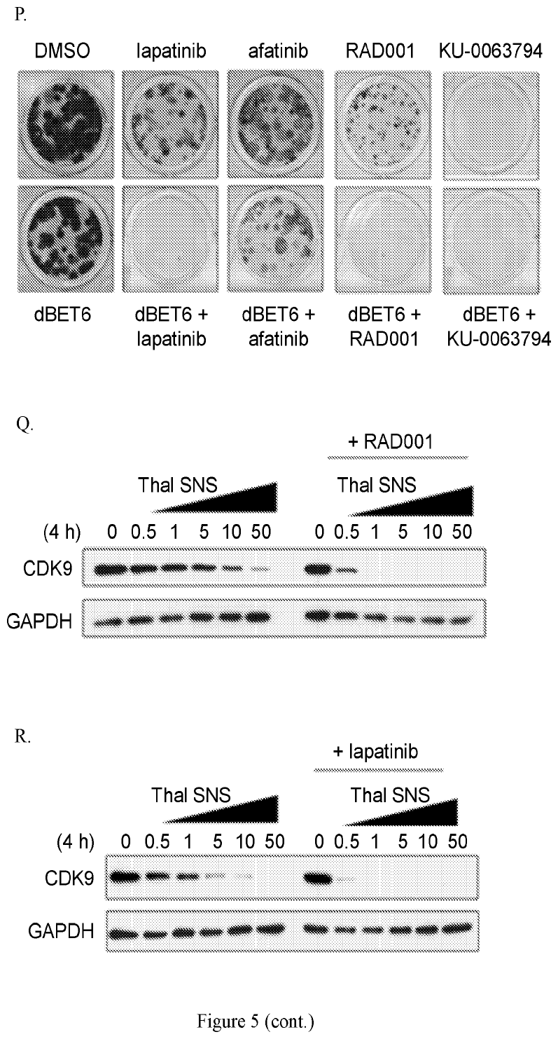

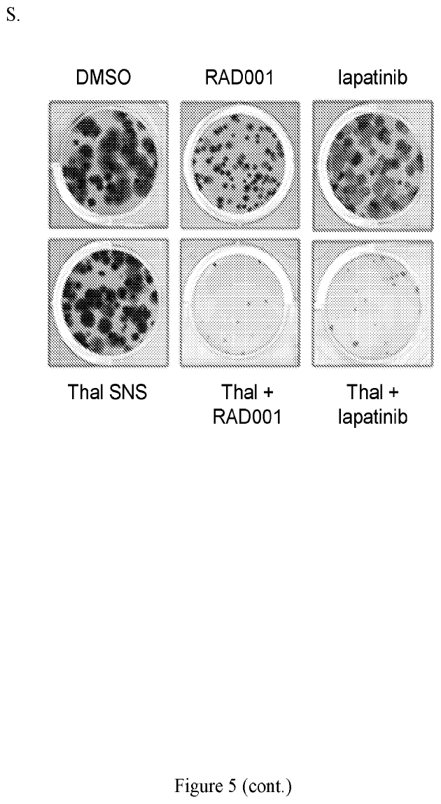

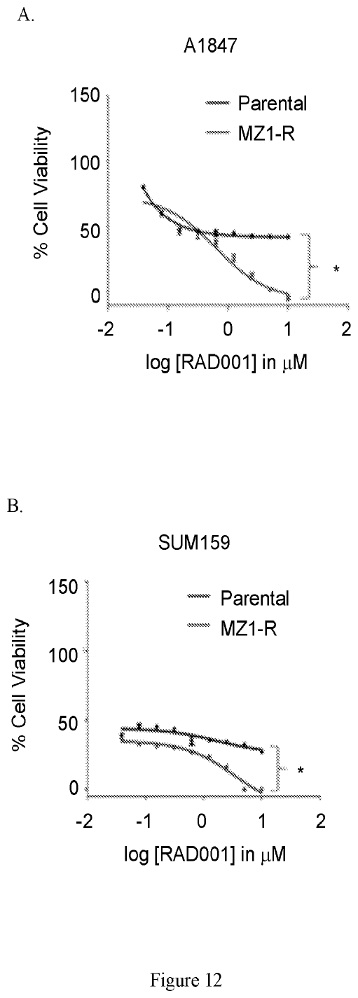

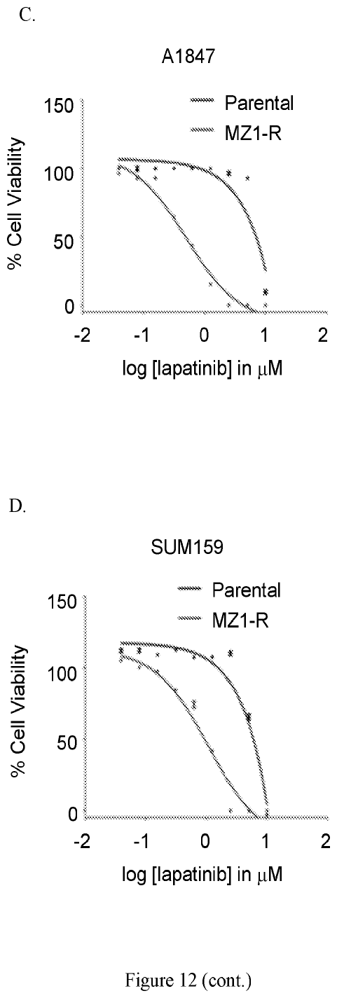

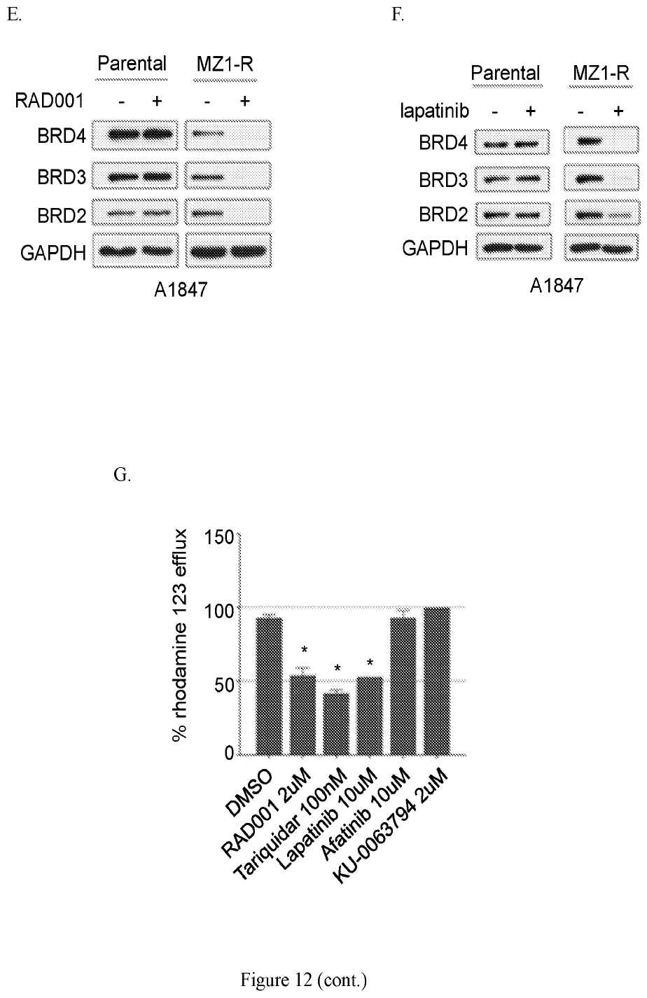

Treatment of degrader-resistant cell lines dBET6-R or Thal-R cell lines with RAD001 or lapatinib reduced MDR1 drug efflux activity similar to that observed with tariquidar (FIG. 5, Panels A and B). Degrader-resistant cell lines were more sensitive to RAD001 (FIG. 5, Panels C and D; FIG. 12, Panels A and B) or lapatinib (FIG. 5, Panels E and F; FIG. 12, Panels C and D) than parental cells and administration of RAD001 or lapatinib resulted in degradation of BET proteins (FIG. 5, Panel G; FIG. 12, Panels E and F) or CDK9 (FIG. 5, Panel H) uniquely in degrader-resistant cell lines. Moreover, treatment of BET protein (FIG. 5, Panel I) or CDK9 (FIG. 5, Panel J) degrader-resistant cell lines with RAD001 or lapatinib resulted in apoptosis similar to tariquidar treatment, demonstrating RAD001 or lapatinib can block MDR1 function overcoming MDR1-driven degrader-resistance.

Next, it was explored whether RAD001 or lapatinib-treatment could sensitize MDR1-overexpressing cells to degrader therapies. Treatment of DLD-1 cells with RAD001 or lapatinib reduced MDR1 drug efflux activity similar to tariquidar-treatment (FIG. 5, Panel K), and immunoblot analysis showed RAD001 or lapatinib treatment improved dBET6-mediated degradation of BRD4 lowering the concentration of dBET6 required to achieve maximal protein degradation (FIG. 5, Panels L and M). Notably, a 100-fold reduction in concentrations of dBET6 were required to degrade BRD4 when combined with RAD001 or lapatinib. In contrast, combined treatment of DLD-1 cells with KU-0063794 (MTOR inhibitor) or afatinib (ErbB receptor inhibitor), drugs that do not inhibit MDR1 function (FIG. 12, Panel G), failed to improve degradation of BRD4 (FIG. 5, Panels N and O). Moreover, treatment of DLD-1 cells with lapatinib but not afatinib sensitized DLD-1 cells to dBET6 providing durable inhibition of colony formation over a 14-day period (FIG. 5, Panel P). Similarly, co-treatment of DLD-1 cells with KU-0063694 and dBET6 did not improve growth inhibition observed with the RAD001 and dBET6 combination, where single agent KU-0063694-treatment completely repressed colony formation. RAD001 or lapatinib-treatment also sensitized DLD-1 cells to Thal SNS 032, improving degradation of CDK9 (FIG. 5, Panel Q and R), and enhancing growth inhibition of colonies (FIG. 5, Panel S). Together, these findings demonstrate RAD001 or lapatinib can be utilized as MDR1 inhibitors to overcome degrader-resistance mediated by MDR1 drug efflux.

### Example 6: Lapatinib-Treatment Enhances MEK1/2 Degrader Therapies in K-Ras Mutant Colorectal Cancer Cells by Dual Blockade of MDR1 Activity and ERBB Receptor Signaling

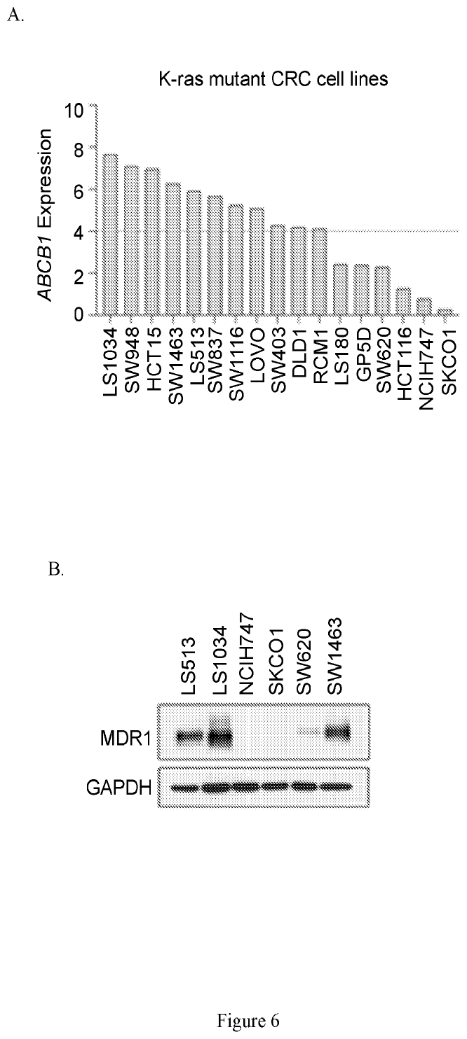

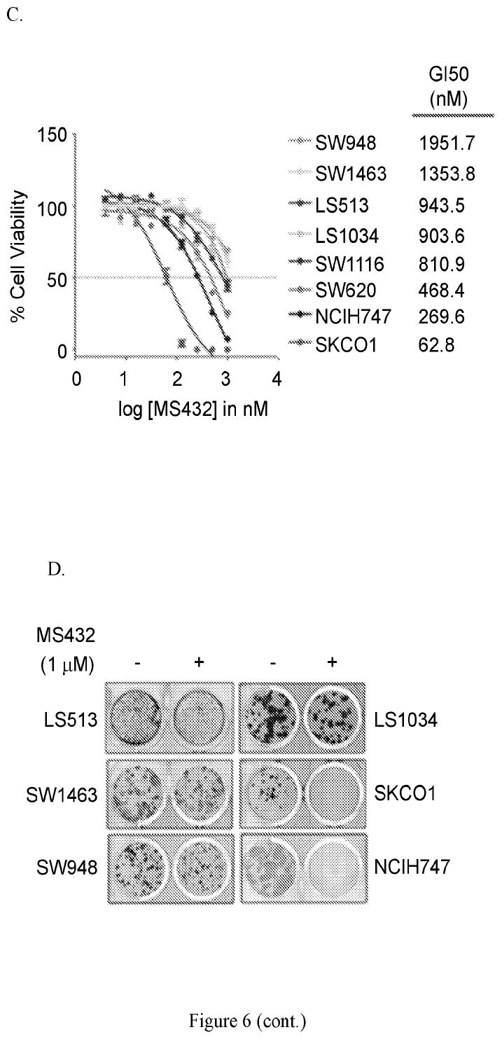

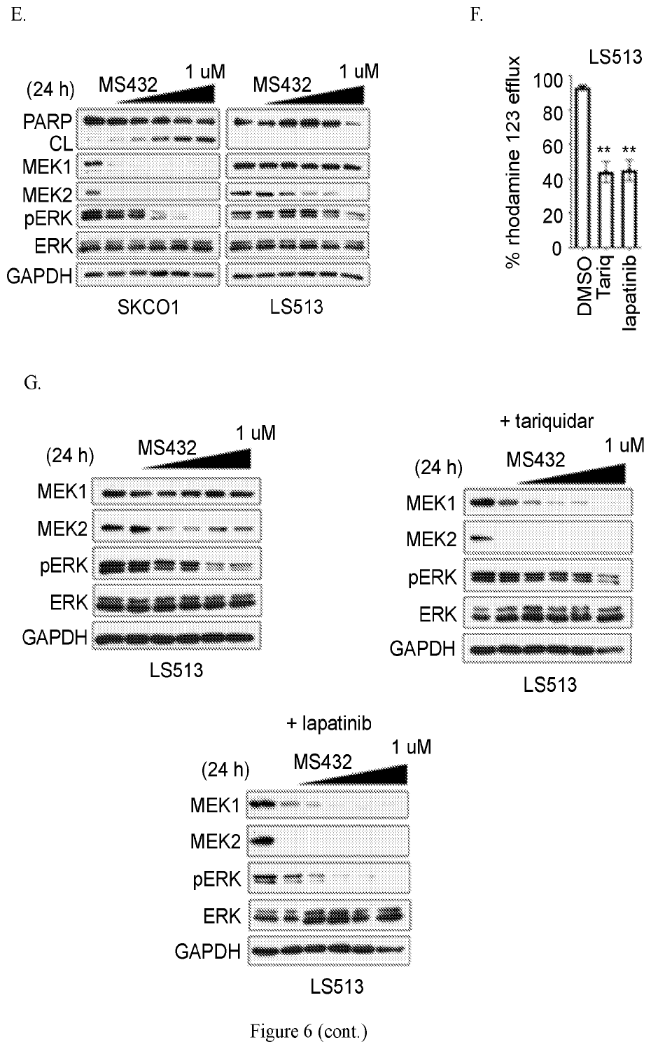

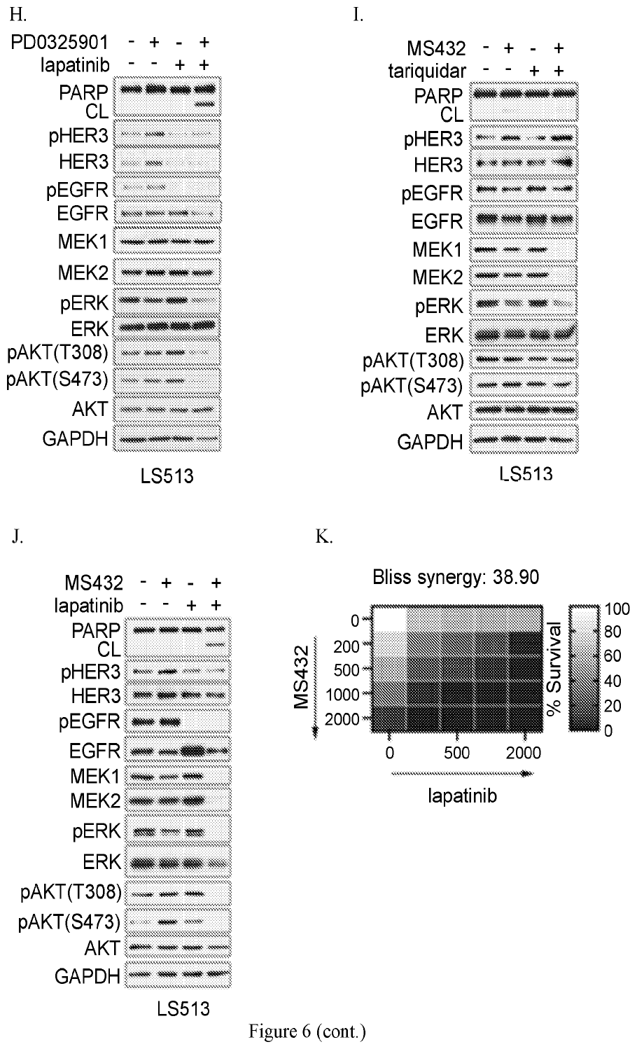

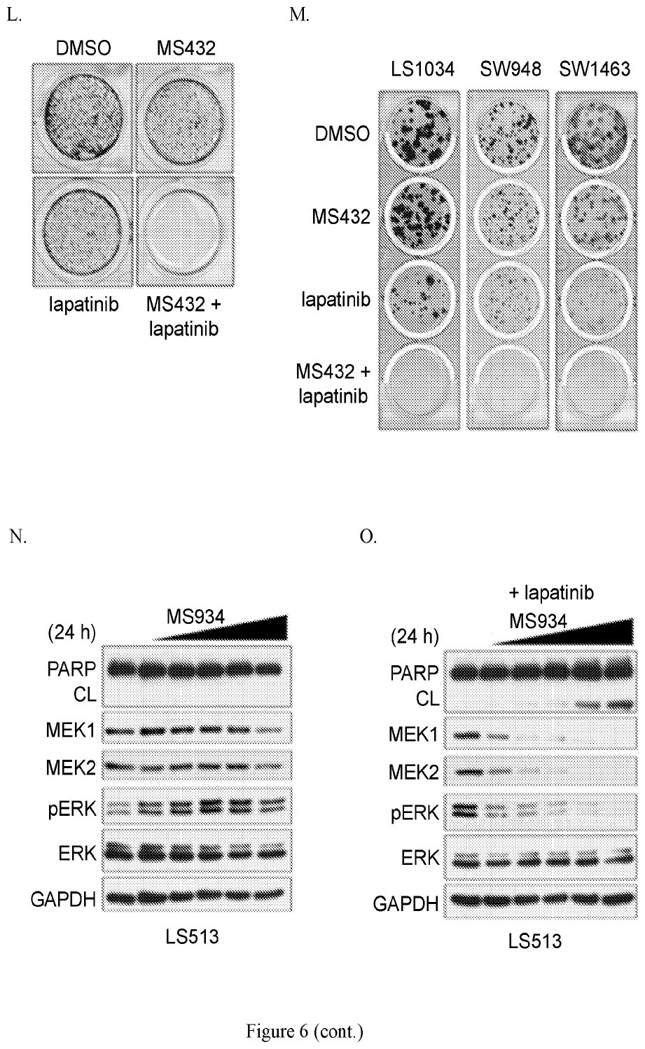

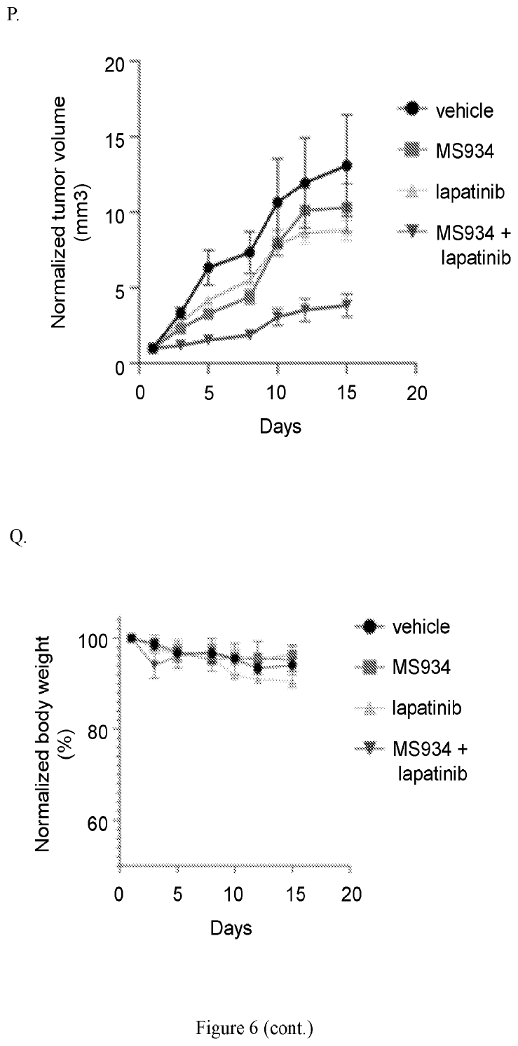

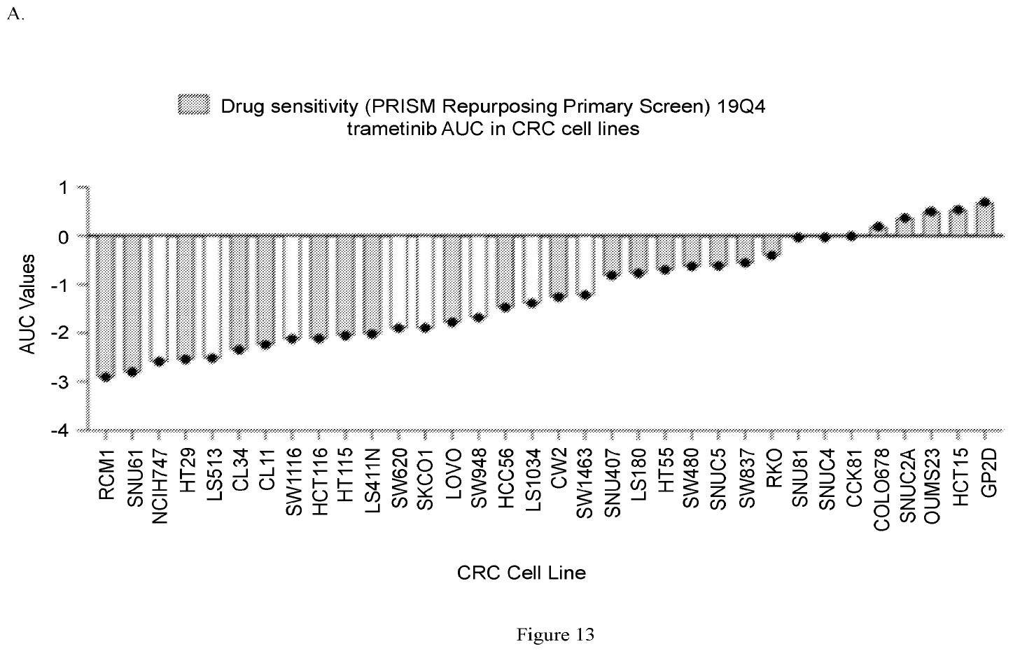

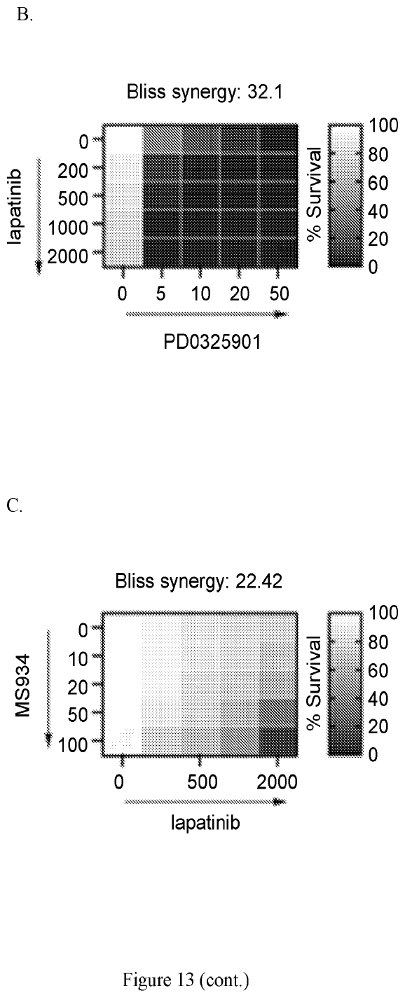

Concomitant blockade of MDR1 may be required to achieve therapeutic efficacy with MEK1/2 degraders (FIG. 6, Panels A and B). MDR1 overexpressing K-ras mutant CRC cell lines (LS1034, LS513, SW948 and SW1463) were more resistant to MEK1/2 degrader MS432, than MDR1 low expressing CRC cell lines (SKCO1, NCIH747, and SW620) (FIG. 6, Panels C and D). Notably, all K-ras mutant cell lines were sensitive to treatment with MEK inhibitor, trametinib (Barretina et al., Nature, 2012, 483, 603-607) (FIG. 13, Panel A). Moreover, treatment of MDR1-overexpressing cell line LS513 with MS432 did not reduce MEKI or MEK2 protein levels, inhibit ERK1/2 phosphorylation or induce apoptosis that was observed with degrader-sensitive MDR1 non-expressing cell line, SKCO1 (FIG. 6, Panel E). Treatment of LS513 cells with lapatinib reduced MDR1 drug efflux activity similar to tariquidar (FIG. 6, Panel F), and co-treatment of LS513 cells with MS432 and lapatinb improved the degradation efficiency of MEKI and MEK2, as well as reduced ERK1/2 phosphorylation at lower concentrations of MS432 (FIG. 6, Panel G). Notably, the addition of lapatinib to MS432 reduced levels of ERK1/2 activating phosphorylation to a greater extent than the tariquidar/MS432 combined treatment, suggesting concurrent blockade of ErbB receptors and MDR1 may be more efficacious than inhibiting MDR1 activity alone.

Next, the impact of blockade of MDR1 alone using tariquidar or both ErbB receptors and MDR1 on kinase signaling in LS513 cells was explored. Treatment of LS513 cells with MEK inhibitors induced ERBB3 and downstream AKT and RAF signaling, which could be blocked by lapatinib treatment (FIG. 6, Panel H), and combining lapatinib and PD0325901 exhibited drug synergy (FIG. 13, Panel B). Notably, co-treatment of LS513 cells with MS432 and lapatinib but not tariquidar reduced MEKi-induced ERBB3 and downstream AKT activation, as well as distinctly induced apoptosis (FIG. 6, Panels I and J). Combination therapies involving MS432 and lapatinib in LS513 cells exhibited robust drug synergy with a Bliss synergy score of 38.9 (FIG. 6, Panel K), as well as provided durable inhibition of colony formation over a 14-day period (FIG. 6, Panel L). Furthermore, the combination of lapatinib and MS432 provided durable growth inhibition of other MDR1-overexpressing K-ras mutant CRC cell lines (FIG. 6, Panel M). Next, the efficacy of combining MEK degraders and lapatinib in vivo using LS513 xenograft models and the recently published MEK degrader MS934, which has optimal bioavailability for animal studies (Hu et al., J. Med. Chem., 2020, 63, 15883-15905) was explored. Similar to MS432, combining MS934 and lapatinib enhanced MEK1/2 degradation in LS513, exhibited drug synergy, and distinctly induced apoptosis (FIG. 6, Panels N and 0; FIG. 13, Panel C). Treatment of mice harboring LS513 xenografts with the MEK degrader MS934 and lapatinib distinctly reduced tumor growth with minimal impact on mice body weight, while single agents were ineffective (FIG. 6, Panels P and Q), suggesting concurrent blockade of ErbB receptors and MDR1 will likely be required to achieve therapeutic response using MEK degraders in K-ras mutant CRC cells.

### Example 7: Combining Lapatinib and KRASG12C Degrader LC-2 Exhibits Drug Synergy in K-Ras G12C Mutant CRC Cells

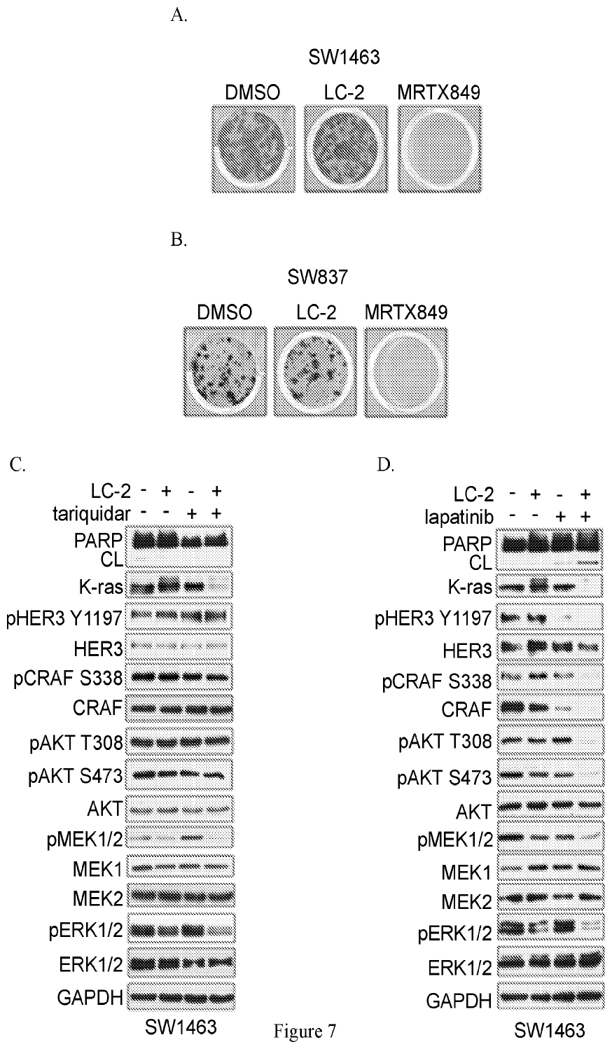

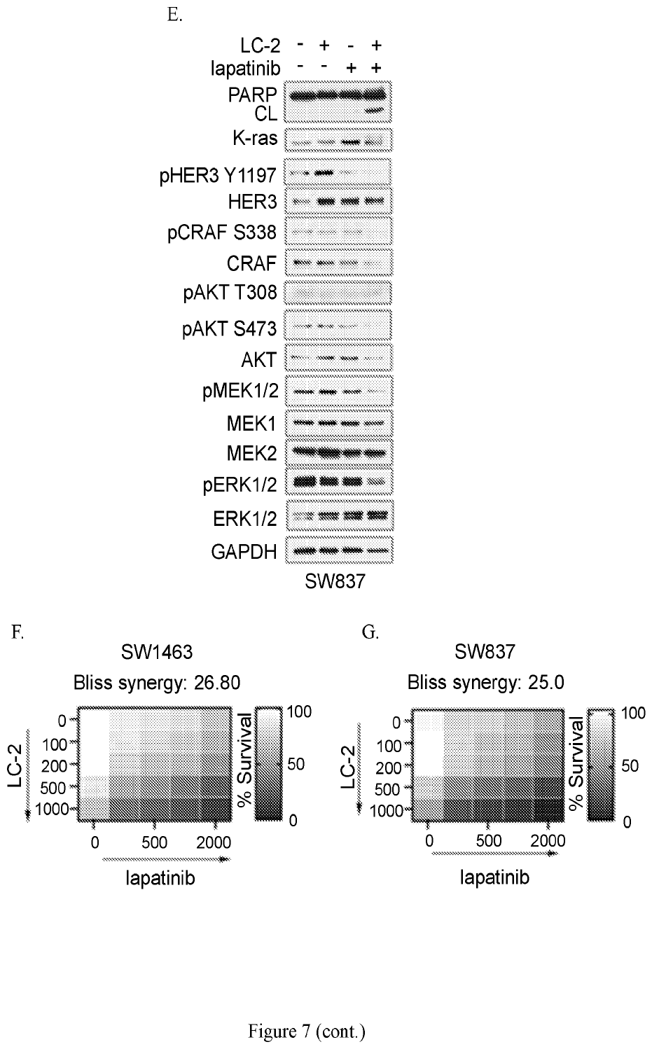

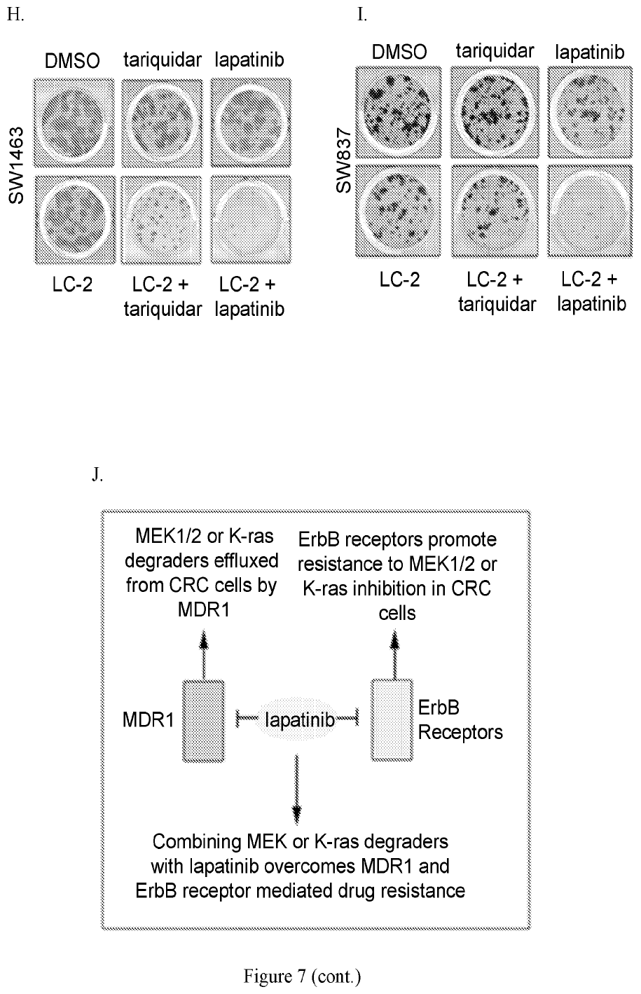

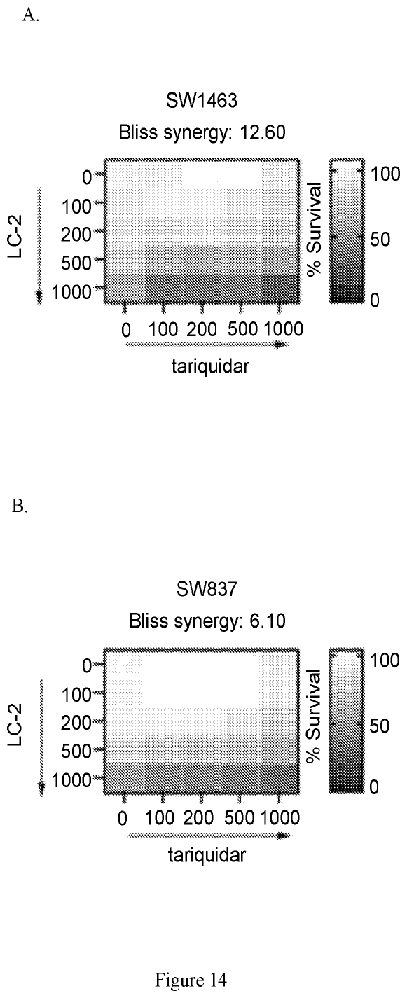

SW1463 or SW837 KRASG12C cells exhibited intrinsic resistance to LC-2 but were sensitive to KRASG12C inhibitor MRTX849 treatment (FIG. 7, Panels A and B). Treatment of SW1463 cells, which harbor a homozygous KRASG12C mutation, with 1 μM of LC-2 had no impact on KRASG12C protein levels, while combining tariquidar or lapatinib with LC-2 improved PROTAC-mediated degradation of KRASG12C reducing protein levels (FIG. 7, Panels C and D). Of particular interest, combining either tariquidar or lapatinib with LC-2 reduced phosphorylation of MEK and ERK, but the lapatinib combination uniquely reduced CRAF and AKT phosphorylation, as well as induced apoptosis. Similarly, treatment of SW837 cells with LC-2 and lapatinib distinctly reduced KRAS effectors CRAF, AKT, MEK and ERK phosphorylation, as well as caused apoptosis (FIG. 7, Panel E). Notably, it was difficult to observe enhanced reduction in KRASG12C protein levels in response to LC-2 and lapatinib treatment in SW837 cells, likely due to SW837 cells being heterozygous for KRASG12C expressing a copy of KRASWT. Combining LC-2 and lapatinib exhibited drug synergy in SW1463 and SW837 with Bliss synergy scores of 26.8 and 25.0 (FIG. 7, Panels F and G), while tariquidar showed marginal synergy in either cell line (FIG. 14, Panels A and B). Furthermore, LC-2 in combination with lapatinib blocked colony formation in SW1463 and SW837 cells to a greater extent than LC-2/tariquidar treatments (FIG. 7, Panels H and I), demonstrating combined blockade of ErbB receptors and MDR1 was required to achieve durable growth inhibition using LC-2 in KRASG12C CRC cells.

Together, these findings suggest the combination of dual MDR1/ErbB receptor inhibitor lapatinb and PROTACs targeting MEK1/2 or KRASG12C represents a promising combination therapy for K-ras mutant CRC cells due to simultaneous blockade of both MDR1 and ErbB receptor driven resistance programs (FIG. 7, Panel J).

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A pharmaceutical composition comprising:
one or more PROTAC therapeutic agents; and
one or more kinase inhibitors, one or more ABC transporter inhibitors, or one or more dual ABC transporter/kinase inhibitors, or any combination thereof.

2. The pharmaceutical composition according to claim 1, comprising one or more PROTAC therapeutic agents, one or more kinase inhibitors, and one or more ABC transporter inhibitors.

3. The pharmaceutical composition according to claim 1, comprising one or more PROTAC therapeutic agents and one or more dual ABC transporter/kinase inhibitors.

4. The pharmaceutical composition according to any one of claims 1 to 3, wherein the PROTAC targets the EGFR-KRAS-PI3K-MEK signaling pathway.

5. The pharmaceutical composition according to claim 4, wherein the PROTAC targets EGFR, HER2, HER3, PI3K, AKT, KRAS, RAF, MEK, or ERK.

6. The pharmaceutical composition according to any one of claims 1 to 5, wherein the kinase inhibitor is an RTK signaling pathway inhibitor.

7. The pharmaceutical composition according to claim 6, wherein the RTK signaling pathway inhibitor is an INSR/IGF1R inhibitor, an EGFR/ERBB/HER2/HER3/HER4 inhibitor, an RTK inhibitor, an MERTK inhibitor, an MST1R inhibitor, or an FGFR1 inhibitor, or any combination thereof.

8. The pharmaceutical composition according to claim 7, wherein the INSR/IGF1R inhibitor is GSK1904529A, the EGFR/ERBB/HER2/HER3/HER4 inhibitor is lapatinib, the RTK inhibitor is imatinib, the MERTK inhibitor is MRX-2843, the MST1R inhibitor is LY2801653 dihydrochloride, and the FGFR1 inhibitor is PD173074.

9. The pharmaceutical composition according to any one of claims 1 to 5, wherein the kinase inhibitor is an MTOR signaling pathway inhibitor.

10. The pharmaceutical composition according to claim 9, wherein the MTOR signaling pathway inhibitor is an mTORC1 inhibitor, a PI3K inhibitor, a P70S6K inhibitor, a PI4K2A inhibitor, or a CDK9 inhibitor, or any combination thereof.

11. The pharmaceutical composition according to claim 10, wherein the mTORC1 inhibitor is RAD001 or Torin-1, the PI3K inhibitor is GDC-0941, the P70S6K inhibitor is LY2584702, the PI4K2A inhibitor is PI-273, and the CDK9 inhibitor is NVP-2.

12. The pharmaceutical composition according to any one of claims 1 to 5, wherein the kinase inhibitor is a CDK7 inhibitor, CK2, AKT1/2, AAK1, FAK1, MEK, and RAF inhibitor.

13. The pharmaceutical composition according to claim 9, wherein the MTOR inhibitor is Dactolisib (BEZ235 or NVP-BEZ235), Rapamycin (Sirolimus), Everolimus (RAD001), AZD8055, Temsirolimus (CCI-779 or NSC 683864), PI-103, KU-0063794, Torkinib (PP242), Tacrolimus (FK506), Ridaforolimus (Deforolimus or MK-8669), Sapanisertib (INK 128, MLN0128, or TAK-228), Voxtalisib (SAR245409 or XL765) Analogue, Torin 1, Omipalisib (GSK2126458 or GSK458), OSI-027, PF-04691502, Apitolisib (GDC-0980 or RG7422), GSK1059615, WYE-354, Gedatolisib (PF-05212384 or PKI-587), Torin 2, WYE-125132 (WYE-132), Vistusertib (AZD2014), BGT226 (NVP-BGT226), Palomid 529 (P529), PP121, WYE-687, WAY-600, ETP-46464, GDC-0349, XL388, Zotarolimus (ABT-578), LY3023414, CC-115, MHY1485, CZ415, GDC-0084, Voxtalisib (XL765 or SAR245409), 3BDO, Bimiralisib (PQR309), CC-223, or SF2523.

14. The pharmaceutical composition according to claim 3, wherein the PROTAC targets the EGFR-KRAS-PI3K-MEK signaling pathway and the dual ABC transporter/kinase inhibitor is lapatinib or RAD001.

15. The pharmaceutical composition according to claim 3, wherein the PROTAC targets the EGFR-KRAS-PI3K-MEK signaling pathway and the dual ABC transporter/kinase inhibitor is lapatinib.

16. The pharmaceutical composition according to claim 14 or claim 15, wherein the PROTAC targets EGFR, HER2, HER3, PI3K, AKT, KRAS, RAF, MEK, or ERK.

17. The pharmaceutical composition according to any one of claims 1 to 16, further comprising one or more KRAS inhibitors and/or one or more autophagy inhibitors.

18. The pharmaceutical composition according to any one of claims 1 to 17, wherein a PROTAC therapeutic agent is linked to a kinase inhibitor in the form of a caged molecule.

19. The pharmaceutical composition according to any one of claims 1 to 17, wherein a PROTAC therapeutic agent is linked to an ABC transporter inhibitor in the form of a caged molecule.

20. The pharmaceutical composition according to any one of claims 1 to 17, wherein a PROTAC therapeutic agent is linked to a dual ABC transporter/kinase inhibitor in the form of a caged molecule.

21. A method for augmenting the therapeutic effect of a human undergoing cancer treatment with a PROTAC therapeutic agent, the method comprising administering one or more kinase inhibitors, one or more ABC transporter inhibitors, or one or more dual ABC transporter/kinase inhibitors, or any combination thereof, to the human.

22. The method according to claim 21, wherein the method comprises administering one or more kinase inhibitors and one or more ABC transporter inhibitors to the human.

23. The method according to claim 21, wherein the method comprises administering one or more dual ABC transporter/kinase inhibitors to the human.

24. The method according to any one of claims 21 to 23, wherein the PROTAC therapeutic agent targets the EGFR-KRAS-PI3K-MEK signaling pathway.

25. The method according to claim 24, wherein the PROTAC therapeutic agent targets EGFR, HER2, HER3, PI3K, AKT, KRAS, RAF, MEK, or ERK.

26. The method according to any one of claims 21 to 25, wherein the kinase inhibitor is an RTK signaling pathway inhibitor.

27. The method according to claim 26, wherein the RTK signaling pathway inhibitor is an INSR/IGF1R inhibitor, an EGFR/ERBB/HER2/HER3/HER4 inhibitor, an RTK inhibitor, an MERTK inhibitor, an MST1R inhibitor, or an FGFR1 inhibitor, or any combination thereof.

28. The method according to claim 27, wherein the INSR/IGF1R inhibitor is GSK1904529A, the EGFR/ERBB/HER2/HER3/HER4 inhibitor is lapatinib, the RTK inhibitor is imatinib, the MERTK inhibitor is MRX-2843, the MST1R inhibitor is LY2801653 dihydrochloride, and the FGFR1 inhibitor is PD173074.

29. The method according to any one of claims 21 to 25, wherein the kinase inhibitor is an MTOR signaling pathway inhibitor.

30. The method according to claim 29, wherein the MTOR signaling pathway inhibitor is an mTORC1 inhibitor, a PI3K inhibitor, a P70S6K inhibitor, a PI4K2A inhibitor, or a CDK9 inhibitor, or any combination thereof.

31. The method according to claim 30, wherein the mTORCT inhibitor is RAD001 or Torin-1, the PI3K inhibitor is GDC-0941, the P70S6K inhibitor is LY2584702, the PI4K2A inhibitor is PI-273, and the CDK9 inhibitor is NVP-2.

32. The method according to any one of claims 21 to 25, wherein the kinase inhibitor is a CDK7 inhibitor, CK2, AKT1/2, AAK1, FAK1, MEK, and RAF inhibitor.

33. The method according to claim 29, wherein the MTOR inhibitor is Dactolisib (BEZ235 or NVP-BEZ235), Rapamycin (Sirolimus), Everolimus (RAD001), AZD8055, Temsirolimus (CCI-779 or NSC 683864), PI-103, KU-0063794, Torkinib (PP242), Tacrolimus (FK506), Ridaforolimus (Deforolimus or MK-8669), Sapanisertib (INK 128, MLN0128, or TAK-228), Voxtalisib (SAR245409 or XL765) Analogue, Torin 1, Omipalisib (GSK2126458 or GSK458), OSI-027, PF-04691502, Apitolisib (GDC-0980 or RG7422), GSK1059615, WYE-354, Gedatolisib (PF-05212384 or PKI-587), Torin 2, WYE-125132 (WYE-132), Vistusertib (AZD2014), BGT226 (NVP-BGT226), Palomid 529 (P529), PP121, WYE-687, WAY-600, ETP-46464, GDC-0349, XL388, Zotarolimus (ABT-578), LY3023414, CC-115, MHY1485, CZ415, GDC-0084, Voxtalisib (XL765 or SAR245409), 3BDO, Bimiralisib (PQR309), CC-223, or SF2523.

34. The method according to claim 23, wherein the PROTAC targets the EGFR-KRAS-PI3K-MEK signaling pathway and the dual ABC transporter/kinase inhibitor is lapatinib or RAD001.

35. The method according to claim 23, wherein the PROTAC targets the EGFR-KRAS-PI3K-MEK signaling pathway and the dual ABC transporter/kinase inhibitor is lapatinib.

36. The method according to claim 34 or claim 35, wherein the PROTAC targets EGFR, HER2, HER3, PI3K, AKT, KRAS, RAF, MEK, or ERK.

37. The method according to any one of claims 21 to 36, further comprising one or more KRAS inhibitors and/or one or more autophagy inhibitors.

38. The method according to any one of claims 21 to 37, wherein the amount of the PROTAC therapeutic agent administered to the human undergoing cancer treatment and receiving administration of one or more kinase inhibitors, one or more ABC transporter inhibitors, or one or more dual ABC transporter/kinase inhibitors, or any combination thereof, is reduced compared to the amount of the PROTAC therapeutic agent administered to a human undergoing cancer treatment in the absence of receiving administration of one or more kinase inhibitors, one or more ABC transporter inhibitors, or one or more dual ABC transporter/kinase inhibitors, or any combination thereof.

39. The method according to any one of claims 21 to 38, wherein the cancer is acute myeloid leukemia, acute monocytic leukemia, prostatic adenocarcinoma, ovarian carcinoma, or epithelial ovarian cancer.

40. The method according to claim 39, wherein the epithelial ovarian cancer is High-Grade Serous Ovarian Carcinoma (HGSOC).

41. The method according to any one of claims 21 to 38, wherein the cancer is a KRAS cancer, PIK3CA cancer, or PTEN cancer.

42. A method of treating cancer in a human in need thereof, the method comprising administering to the human:
one or more PROTAC therapeutic agents; and
one or more kinase inhibitors, one or more ABC transporter inhibitors, or one or more dual ABC transporter/kinase inhibitors, or any combination thereof.

43. The method according to claim 42, wherein the human is administered one or more PROTAC therapeutic agents, one or more kinase inhibitors, and one or more ABC transporter inhibitors.

44. The method according to claim 42, wherein the human is administered one or more PROTAC therapeutic agents and one or more dual ABC transporter/kinase inhibitors.

45. The method according to any one of claims 42 to 44, wherein the PROTAC targets the EGFR-KRAS-PI3K-MEK signaling pathway.

46. The method according to claim 45, wherein the PROTAC targets EGFR, HER2, HER3, PI3K, AKT, KRAS, RAF, MEK, or ERK.

47. The method according to any one of claims 42 to 46, wherein the kinase inhibitor is an RTK signaling pathway inhibitor.

48. The method according to claim 47, wherein the RTK signaling pathway inhibitor is an INSR/IGF1R inhibitor, an EGFR/ERBB/HER2/HER3/HER4 inhibitor, an RTK inhibitor, an MERTK inhibitor, an MST1R inhibitor, or an FGFR1 inhibitor, or any combination thereof.

49. The method according to claim 48, wherein the INSR/IGF1R inhibitor is GSK1904529A, the EGFR/ERBB/HER2/HER3/HER4 inhibitor is lapatinib, the RTK inhibitor is imatinib, the MERTK inhibitor is MRX-2843, the MST1R inhibitor is LY2801653 dihydrochloride, and the FGFR1 inhibitor is PD173074.

50. The method according to any one of claims 42 to 46, wherein the kinase inhibitor is an MTOR signaling pathway inhibitor.

51. The method according to claim 50, wherein the MTOR signaling pathway inhibitor is an mTORC1 inhibitor, a P13K inhibitor, a P70S6K inhibitor, a PI4K2A inhibitor, or a CDK9 inhibitor, or any combination thereof.

52. The method according to claim 51, wherein the mTORC1 inhibitor is RAD001 or Torin-1, the PI3K inhibitor is GDC-0941, the P70S6K inhibitor is LY2584702, the PI4K2A inhibitor is PI-273, and the CDK9 inhibitor is NVP-2.

53. The method according to any one of claims 42 to 46, wherein the kinase inhibitor is a CDK7 inhibitor, CK2, AKT1/2, AAK1, FAK1, MEK, and RAF inhibitor.

54. The method according to claim 50, wherein the MTOR inhibitor is Dactolisib (BEZ235 or NVP-BEZ235), Rapamycin (Sirolimus), Everolimus (RAD001), AZD8055, Temsirolimus (CCI-779 or NSC 683864), PI-103, KU-0063794, Torkinib (PP242), Tacrolimus (FK506), Ridaforolimus (Deforolimus or MK-8669), Sapanisertib (INK 128, MLN0128, or TAK-228), Voxtalisib (SAR245409 or XL765) Analogue, Torin 1, Omipalisib (GSK2126458 or GSK458), OSI-027, PF-04691502, Apitolisib (GDC-0980 or RG7422), GSK1059615, WYE-354, Gedatolisib (PF-05212384 or PKI-587), Torin 2, WYE-125132 (WYE-132), Vistusertib (AZD2014), BGT226 (NVP-BGT226), Palomid 529 (P529), PP121, WYE-687, WAY-600, ETP-46464, GDC-0349, XL388, Zotarolimus (ABT-578), LY3023414, CC-115, MHY1485, CZ415, GDC-0084, Voxtalisib (XL765 or SAR245409), 3BDO, Bimiralisib (PQR309), CC-223, or SF2523.

55. The method according to claim 44, wherein the PROTAC targets the EGFR-KRAS-PI3K-MEK signaling pathway and the dual ABC transporter/kinase inhibitor is lapatinib or RAD001.

56. The method according to claim 44, wherein the PROTAC targets the EGFR-KRAS-PI3K-MEK signaling pathway and the dual ABC transporter/kinase inhibitor is lapatinib.

57. The method according to claim 55 or claim 56, wherein the PROTAC targets EGFR, HER2, HER3, PI3K, AKT, KRAS, RAF, MEK, or ERK.

58. The method according to any one of claims 42 to 57, further comprising administering one or more KRAS inhibitors and/or one or more autophagy inhibitors to the human.

59. The method according to any one of claims 42 to 58, wherein the amount of the PROTAC therapeutic agent administered to the human receiving administration of one or more kinase inhibitors, one or more ABC transporter inhibitors, or one or more dual ABC transporter/kinase inhibitors, or any combination thereof, is reduced compared to the amount of the PROTAC therapeutic agent administered to a human undergoing cancer treatment in the absence of receiving administration of one or more kinase inhibitors, one or more ABC transporter inhibitors, or one or more dual ABC transporter/kinase inhibitors, or any combination thereof.

60. The method according to any one of claims 42 to 59, wherein the cancer is acute myeloid leukemia, acute monocytic leukemia, prostatic adenocarcinoma, ovarian carcinoma, or epithelial ovarian cancer.

61. The method according to claim 60, wherein the epithelial ovarian cancer is High-Grade Serous Ovarian Carcinoma (HGSOC).

62. The method according to any one of claims 42 to 59, wherein the cancer is a KRAS cancer, PIK3CA cancer, or PTEN cancer.

63. The method according to any one of claims 42 to 62, wherein MDR1 is overexpressed in the human.

64. The method according to any one of claims 42 to 63, wherein a PROTAC therapeutic agent is linked to a kinase inhibitor in the form of a caged molecule.

65. The method according to any one of claims 42 to 63, wherein a PROTAC therapeutic agent is linked to an ABC transporter inhibitor in the form of a caged molecule.

66. The method according to any one of claims 42 to 63, wherein a PROTAC therapeutic agent is linked to a dual ABC transporter/kinase inhibitor in the form of a caged molecule.
